(19) United States
(12) Patent Application Publication
(10) Pub. No.: US 2026/0055191 A1
(43) Pub. Date: Feb. 26, 2026

(54) GLYCOENGINEERED POLYPEPTIDES TARGETING IMMUNOGLOBULIN A AND COMPLEXES COMPRISING THE SAME

(71) Applicant: GlycoEra AG, Wädenswil (CH)

(72) Inventors: Ganesh Venkataraman Kaundinya, Bedford, MA (US); Tanmoy Chinmoy Ganguly, Westford, MA (US); Rainer Follador, Chur (CH); Manuela Mally, Watt (CH); Jonathan Albert Back, Villers-Le-Lac (FR); Michela Manni, Zürich (CH)

(21) Appl. No.: 19/144,981

(22) PCT Filed: Jan. 5, 2024
(86) PCT No.: PCT/IB2024/050113

§ 371 Date: Jul. 1, 2025

Related U.S. Application Data
(60) Provisional application No. 63/478,578, filed on Jan. 5, 2023.

(51) Int. Cl. C07K 16/28; A61K 39/00; A61K 45/06; A61P 31/06; C12N 1/10; C12R 1/90

(52) U.S. Cl. CPC C07K 16/283; A61K 45/06; A61P 31/06; C07K 16/28; C12N 1/10; A61K 2039/505; A61K 2039/54; C07K 2317/14; C07K 2317/31; C07K 2317/41; C07K 2317/76; C07K 2317/77; C07K 2319/02; C07K 2319/50; C12R 2001/90

(57) ABSTRACT

Provided herein are glycoengineered polypeptides comprising a first moiety comprising one or more peptides that specifically binds to a target antibody (e.g., an IgA1 or an immune complex comprising the same, a gd-IgA1 or an immune complex comprising the same, or an anti-gd-IgA1 autoantibody or an immune complex comprising the same) and a second moiety comprising one or more glycans conjugated to the first moiety at one or more glycosylation sites. Also provided herein are nucleic acid sequence encoding provided glycoengineered polypeptides. Further provided herein are compositions comprising glycoengineered polypeptides and/or nucleic acids encoding the same, as well as methods of making and using the same.

### CROSS REFERENCE TO RELATED APPLICATIONS

This application claims priority to and the benefit of U.S. Provisional Patent Application No. 63/478,578 filed on Jan. 5, 2023, the entire contents of which are hereby incorporated by reference in its entirety.

### BACKGROUND

IgA nephropathy (IgAN) is commonly associated with the accumulation of IgA immune complexes in renal tissue. The IgA immune complexes (also referred to as renal deposits) can include IgA antibodies and complement components and are often implicated in disease pathogenesis.

### SUMMARY

The present disclosure identifies certain challenges with existing therapies used to treat diseases associated with increased and/or aberrant IgA (e.g., IgA1 Nephropathy, or IgAN). For example, the present disclosure identifies limitations of some of the most widely used treatments options. Blood pressure medications and therapies for managing proteinuria are some of the most common therapies for treating diseases associated with increased and/or aberrant IgA (e.g., IgAN). However, these therapeutic options do not directly act on and/or reduce: (1) IgA1 levels or immune complexes comprising the same, (2) galactose-deficient IgA1 (gd-IgA1) levels or immune complexes comprising the same; or (3) anti-gd-IgA1 autoantibodies or immune complexes comprising the same; (4) immune complexes comprising IgA, or (5) antigenic variants of IgG. Therefore, while the currently available treatment options may provide some relief to patients, these treatment options do not treat the underlying cause of disease.

The present disclosure also identifies limitations in certain non-specific B-cell directed therapies proposed for the treatment of IgAN. For example, Atacicept is a fusion protein which non-specifically inhibits B cells by blocking activation of B cells (see, e.g., Hans-Peter Hartung and Bernd C. Kieseier, Ther Adv Neurol Disord. 2010 July; 3(4): 205-216). Atacicept results in non-specific depletion of B cells including antibody producing plasma cells thus reducing the levels of all antibodies in a subject. This non-specific depletion of B cells is not desirable and can lead to unwanted side effects such as organ damage or immunosuppression. Additionally, non-specific therapies such as Atacicept likely need to be dosed for a longer period of time to achieve a desired result, e.g., a clinical response. Furthermore, non-specific therapies such as Atacicept likely cannot produce rapid responses due to the lack of specificity and lack of targeting of disease-causing components, e.g., gd-IgA1 or immune complexes comprising the same, IgA1 or immune complexes comprising the same, or anti-gd-IgA1 or immune complexes comprising the same. Such non-specific therapies are also not able to disrupt and/or remove pathogenic immune complexes.

Without wishing to be bound by any particular theory, the technologies provided in the present disclosure can address certain limitations identified in existing therapies used to treat diseases associated with increased and/or aberrant IgA such as Atacicept. Technologies provided herein specifically target autoantigens or immune complexes comprising the same, or autoantibodies or immune complexes comprising the same, and produce rapid, specific, and durable responses when administered to a subject. In some embodiments, technologies provided herein are anticipated to produce fewer and/or less severe unwanted side effects than non-specific B-cell directed therapies, due to the specificity in targeting immunogenic autoantigens and/or autoantibodies. In some embodiments, technologies provided herein can result in responses (e.g., clinical responses) in a shorter amount of time, as compared to non-specific B-cell directed therapies. In some embodiments, technologies provided herein do not need to be dosed for as long an extended period of time, e.g., as compared to non-specific B-cell directed therapies. In some embodiments, technologies provided herein can disrupt and/or remove immune complexes comprising autoantigens and/or autoantibodies disclosed here. In some embodiments, technologies provided herein can result in improved depletion and/or removal of a specific autoantigen or autoantibody, or immune complexes comprising the same, e.g., gd-IgA1 and/or anti-gd-IgA1, as compared to non-specific B-cell directed therapies. Advantages associated with technologies disclosed herein can result in improved responses (e.g., clinical responses) in patients having or at risk of having diseases associated with increased and/or aberrant IgA (e.g., IgAN).

Among other things, the present disclosure provides technologies for degrading and/or removing immunogenic autoantigens, thereby depleting, reducing and/or removing: (1) IgA1 levels or immune complexes comprising the same, (2) galactose-deficient IgA1 (gd-IgA1) levels or immune complexes comprising the same; or (3) anti-gd-IgA1 autoantibodies or immune complexes comprising the same; (4) immune complexes comprising IgA; or (5) antigenic variants of IgG, by providing glycoengineered polypeptides that can simultaneously bind to a target antibody (e.g., an IgA1 or an immune complex comprising the same, a gd-IgA1 or an immune complex comprising the same, or an anti-gd-IgA1 autoantibody or an immune complex comprising the same) and to an endocytic receptor via one or more glycans. Without wishing to be bound by theory, the present disclosure proposes that binding of a glycoengineered polypeptide disclosed herein to a target antibody and to an endocytic receptor induces internalization of the target antibody into a cell. In some embodiments, internalization of the target antibody results in degradation. The technologies disclosed herein also relate to nucleic acid molecules encoding glycoengineered polypeptides disclosed herein. Further provided herein are compositions comprising glycoengineered polypeptides disclosed herein or nucleic acid molecules encoding the same, and methods of making the same.

Technologies provided herein are particularly useful for reducing and/or removing target antibodies in subjects having, or at risk of having diseases associated with increased and/or aberrant IgA (e.g., IgAN). Administration of compositions comprising glycoengineered polypeptides disclosed herein or nucleic acid molecules encoding the same can reduce and/or deplete target antibodies, thus treating the disease or ameliorating one or more symptoms of the disease.

Accordingly, the present disclosure provides a glycoengineered polypeptide comprising: (a) a first moiety comprising one or more peptides that specifically binds to a target antibody or a fragment or a complex thereof; and (b) a second moiety comprising one or more glycans conjugated to the first moiety at one or more glycosylation sites.

In some embodiments, a complex comprising a target antibody is or comprises an immune complex.

In some embodiments, a target antibody comprises galactose-deficient IgA1 (gd-IgA1), or a fragment or a complex thereof.

In some embodiments, a target antibody comprises IgA1, or a fragment or a complex thereof.

In some embodiments, a target antibody comprises an autoantibody that specifically binds to gd-IgA1 (“anti-gd-IgA1 autoantibody”), or a fragment or a complex thereof.

In some embodiments, a second moiety specifically binds to one or more endocytic receptors. In some embodiments, an endocytic receptor is or comprises an endocytic lectin. In some embodiments, an endocytic receptor is chosen from: an asialoglycoprotein receptor (ASGPR); a mannose binding receptor, a Cluster of Differentiation 206 (CD206) receptor; a DC-SIGN (Cluster of Differentiation 209 or CD209) receptor; a C-Type Lectin Domain Family 4 Member G (LSECTin) receptor; a macrophage inducible Ca2+-dependent lectin receptor (Mincle); a L-SIGN CD209L receptor; dectin-1; dectin-2, langerin, macrophage mannose 2 receptor, BDCA-2, DCIR, MBL, MDL, MICL, CLEC2, CLEC10, DNGR1, CLEC12B, DEC-205, and mannose 6 phosphate receptor (M6PR), or a combination thereof.

In some embodiments, a glycan structure comprises a biantennary GalNAc.

In some embodiments, a biantennary GalNac binds to an asialoglycoprotein receptor (ASGPR) or a fragment or variant thereof, or a complex comprising ASGPR.

The disclosure also provides a composition comprising: (i) a first glycoengineered polypeptide comprising a first moiety that specifically binds to IgA1 or a fragment or a complex thereof, (ii) a second glycoengineered polypeptide comprising a first moiety that specifically binds to gd-IgA1 or a fragment or a complex thereof; and/or (iii) a third glycoengineered comprising a first moiety that specifically binds to an anti-gd-IgA1 autoantibody or a fragment or a complex thereof. In some embodiments, a composition is a pharmaceutical composition.

Further provided herein is a method comprising administering to a subject a pharmaceutical composition comprising a glycoengineered polypeptide disclosed herein or a nucleic acid encoding the same.

Provided herein is a method comprising, assessing a level of a target antibody in a sample from a subject, and administering a pharmaceutical composition comprising a glycoengineered polypeptide disclosed herein or a nucleic acid encoding the same if the level of the target antibody is higher than a comparator.

In some embodiments, a comparator comprises a predetermined reference sample such as a sample obtained from an otherwise similar subject who does not have a disease or disorder, or a symptom of a disease or disorder.

In some embodiments, a subject has or is diagnosed as having a disease associated with increased and/or aberrant IgA. In some embodiments, a disease associated with increased and/or aberrant IgA is IgA nephropathy. In some embodiments, a disease associated with increased and/or aberrant IgA is dermatitis herpetiformis. In some embodiments, a disease associated with increased and/or aberrant IgA is and Henoch-Schoenlein purpura.

In some embodiments, a method is a treatment method.

In some embodiments, a method is a prevention method.

Additional features of glycoengineered polypeptides disclosed herein, nucleic acids encoding the same, composition comprising glycoengineered polypeptides or nucleic acids encoding the same, and methods of making and using the same are provided throughout the present disclosure.

### BRIEF DESCRIPTION OF THE DRAWING

FIG. 1 depicts exemplary configurations of glycoengineered polypeptide constructs described herein. For example, the configurations can be used in glycoengineered polypeptides that specifically bind to IgA1, gd-IgA1, or anti-gd-IgA1. Exemplary configurations depicted in 5′ to 3′ orientation are: (1) VHH-glycotag-cleavable HIS Tag; (2) HIS tag-glycotag-VHH; (3) VHH-glycotag; (4) glycotag-VHH; (5) VHH-half Fc-glycotag; (6) VHH-albumin domain-glycotag; (7) albumin domain-VHH-glycotag; (8) VHH-VHH-glycotag; (9) glycotag-VHH-VHH; (10) VHH-VHH-glycotag-HIS tag; (11) His tag-glycotag-VHH-VHH.

FIG. 2 depicts exemplary configurations of glycoengineered polypeptide constructs described herein. For example, the configurations can be used in glycoengineered polypeptides comprising CD89 polypeptides or fragments thereof, which specifically bind to IgA1. The glycoengineered polypeptides can include native N-glycosylation sites, mutations at native N-glycosylation sites, engineered N-glycosylation sites (glycotag) or any combination thereof.

FIG. 3 is a schematic representation of the depletion assay protocol for experiments in Example 1. Female Wistar rats were injected intravenously with human IgA at −0.5 hours, followed by a subcutaneous injection of 2.0 mg of an exemplary glycoengineered polypeptide of soluble CD89 (labelled G-LyTAC in figure; four rats) or a control PBS solution (four rats) at 0 hours. Serum levels of total (free+bound) human IgA were quantified at 1-, 3-, 6-, 10-, 24-, and 48-hours post-treatment with the sCD89 glycoengineered polypeptide or PBS.

FIG. 4 depicts the depletion of IgA antibodies in rat serum using an exemplary glycoengineered polypeptide of soluble CD89 in the manner depicted in FIG. 3. Results are expressed as a percentage of human IgA antibodies remaining in serum following treatment with the sCD89 glycoengineered polypeptide (triangle markers) or PBS vehicle (circle markers) at each timepoint.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

IgA1: The term “IgA1” is used herein in reference to immunoglobulin A1 polypeptides as understood in the art. IgA1 belongs to the IgA family and is the predominant immunoglobulin produced at mucosal membranes. IgA1 protein is encoded by the IGHA1 gene. Amino acid sequences for full-length IgA1 and/or for nucleic acids that encode it can be found in a public database such as GenBank, UniProt and Swiss-Prot. For example, an amino acid sequence of human IgA1 constant region (SEQ ID NO: 1) can be found as UniProt/Swiss-Prot Accession No. P01876; a nucleic acid sequence encoding human IgA1 can be found readily by one with skill in the art. Those skilled in the art will appreciate that the sequence presented in SEQ ID NO: 1 is exemplary, and certain variations (including, for example, conservative substitutions in SEQ ID NO:1, codon-optimized variants of a nucleic acid sequence encoding human IgA1 etc) are understood to also be or encode human IgA1; additionally, those skilled in the art will appreciate that homologs and orthologs of human IgA1 are known and/or knowable through the exercise of ordinary skill and which may be useful in the present invention, for example, based on degree of sequence identity, presence of one or more characteristic sequence elements, and/or one or more shared activities.

gd-IgA1: As used herein, the term “gd-IgA1” or “galactose-deficient IgA1” refers to IgA1 having a glycan profile that is different from a reference glycan profile of an IgA1. A reference glycan profile can be that of an IgA1 from a healthy individual or a subject who is not at risk of having IgA nephropathy. In some embodiments, a reference glycan profile comprises 1, 2, 3, 4, 5, or 6 O-glycan chains. In some embodiments, gd-IgA1 has reduced glycosylation at a hinge region as compared to IgA1 hinge region glycosylation in a healthy individual or a subject who is not at risk of having IgA nephropathy. In some embodiments, reduced glycosylation comprises reduced O-linked glycosylation. In some embodiments, reduced glycosylation comprises reduced galactosylation. In some embodiments, reduced glycosylation comprises altered glycosylation that comprises fewer galactose moieties as compared to IgA1 glycosylation in a healthy individual or a subject who is not at risk of having IgA nephropathy. In some embodiments, gd-IgA1 has a glycan profile comprising a terminal GalNac. In some embodiments, gd-IgA1 has a glycan profile comprising a terminal GalNac with an alpha2,6 linked sialic acid. In some embodiments, sialylation of a terminal GalNac blocks effective galactosylation.

Glycans: As used herein, the term “glycan” refers to one or more saccharides or sugar chains that can be attached to a protein or lipid to form a glycoconjugate. A glycan conjugated to a protein forms a glycoprotein. A glycan conjugated to a nitrogen atom of an amino acid residue is an N-linked glycan and a glycan conjugated to an oxygen atom of an amino acid residue is an O-linked glycan. As will be appreciated by those of ordinary skill in the art, the structure of a glycan indicates if a specific glycan is an N-linked glycan.

Glycoengineered: As used herein, the term “glycoengineered,” or an equivalent thereof means a process of glycosylating a target protein (e.g., a glycoengineered polypeptide disclosed herein), or a target protein made by such process. In some embodiments, the process uses a host cell system that has one or more enzymes (e.g., pathways) that provides for glycosylation of the target protein; in some other embodiments, the process is performed by chemically attaching one or more glycans to a target protein, e.g., using Click chemistry. Such a host cell system can be genetically engineered to introduce a glycosylation pathway to selectively glycosylate a target protein with a particular glycan structure. A host cell used to generate a glycoengineered target protein can include, for example, a recombinant nucleic acid encoding a target protein; and a recombinant nucleic acid encoding a heterologous glycosyltransferase. The host cell system used for glycoengineering (e.g., to generate a glycoengineered protein) can introduce, eliminate, and/or modify N-linked glycosylation. The host cell system used for glycoengineering (e.g. to generate a glycoengineered protein) can introduce, eliminate, and/or modify O-linked glycosylation. The host cell used for glycoengineering or to generate a glycoengineered target protein can be a mammalian cell, an insect cell, a yeast cell, a bacterial cell, a plant cell, a microalgae, or a protozoa. The protozoa used for glycoengineering can be a species of Leishmania. A glycoengineered target protein also includes a target protein that has been engineered to be selectively glycosylated at one or more specific sites when generated in the host cell system.

Glycoengineered polypeptide: As used herein, a “glycoengineered polypeptide” is a polypeptide that mediates the internalization and/or degradation of a target protein by specifically binding to a target protein (e.g., a target antibody) and engaging with one or more endocytic receptors. In some embodiments, binding (e.g., simultaneous binding) of a glycoengineered polypeptide to a target protein and an endocytic receptor internalizes a target protein and/or activates one or more degradation pathways.

Glycosylation site: As used herein, the term “glycosylation site” refers to a site of glycosylation in a protein. Such a glycosylation site, also referred to as a glycosite herein, can be naturally present in the amino acid sequence of a protein or recombinantly engineered into the protein by addition or substitution or deletion of amino acids. In some embodiments, a glycosylation site is present in a so-called glycotag that is fused to a glycoengineered polypeptide disclosed herein. In certain embodiments, a glycotag is fused to a protein to create a bispecific binding protein. As used herein a glycotag refers to a peptide containing consensus N-glycosylation site sequence fused to N- or a C-terminal or both termini of a protein or polypeptide. In some embodiments, the glycotag is fused to the C-terminus of the of the glycoengineered polypeptide disclosed herein via a peptide linker. In some embodiments, the glycotag is fused to the N-terminus of the glycoengineered polypeptide disclosed herein via a peptide linker. In some embodiments, the peptide linker is a consensus peptide sequence. In some embodiments, the consensus peptide sequence is 1, 2, 3, 4, 5, 6, 7 or more amino acid residues in length. In some embodiments, the bifunctional protein provided herein contains 1, 2, 3, 4, 5, 6, 7, 8, 9, 10 or more glycotags.

Endocytic receptor: As used herein, the term “endocytic receptor” refers to a receptor or a fragment thereof that binds to a target and internalizes the target into a cell. In some embodiments, an endocytic receptor recognizes and binds to one or more glycans on a target. In some embodiments, binding of an endocytic receptor to a target internalizes the target into a cell, e.g., into a lysosome or phagosome. In some embodiments, an endocytic receptor is or comprises an endocytic lectin. In some embodiments, an endocytic receptor is chosen from: an asialoglycoprotein receptor (ASGPR); a mannose binding receptor, a Cluster of Differentiation 206 (CD206) receptor; a DC-SIGN (Cluster of Differentiation 209 or CD209) receptor; a C-Type Lectin Domain Family 4 Member G (LSECTin) receptor; a macrophage inducible Ca2+-dependent lectin receptor (Mincle); a L-SIGN CD209L receptor; dectin-1; dectin-2, langerin, macrophage mannose 2 receptor, BDCA-2, DCIR, MBL, MDL, MICL, CLEC2, CLEC10, DNGR1, CLEC12B, DEC-205, and mannose 6 phosphate receptor (M6PR), or a combination thereof.

Target antibody: As used herein, the term “target antibody” refers to: (1) an IgA1, a fragment thereof, or an immune complex comprising the same; (2) a galactose-deficient IgA1 (gd-IgA1) a fragment thereof, or an immune complex comprising the same; (3) an anti-gd-IgA1 autoantibody a fragment thereof, or an immune complex comprising the same; (4) immune complexes comprising IgA, or (5) antigenic variants of IgG. In some embodiments, an immune complex comprises one or more antibodies, an antigen recognized by one or more antibodies or autoantibodies, and/or one or more components of a complement system. In some embodiments, a complement component comprises: C3, C5b, C6, C7, C8, and/or C9, or fragments of any complement component or combinations thereof. In some embodiments, an immune complex comprises C3 or fragments of C3. In some embodiments, C3 fragments comprises iC3b, C3c, C3dg, or combinations thereof. In some embodiments, an immune complex comprises one or more antibodies chosen from: an IgG, an IgA, an IgM, an IgD, an IgE, or fragments or combinations thereof. In some embodiments, a target antibody comprises a gd-IgA1 antibody, a fragment thereof, or an immune complex comprising the same. In some embodiments, a target antibody comprises an IgA1 antibody, a fragment thereof, or an immune complex comprising the same. In some embodiments, a target antibody comprises an anti-gd-IgA1 autoantibody, a fragment thereof, or an immune complex comprising the same. In some embodiments, an anti-gd-IgA1 autoantibody is an IgG or an IgM. In some embodiments, a target antibody comprises one or more antigenic variants of IgG, e.g., as disclosed herein.

Disease associated with increased and/or aberrant IgA: As used herein, the term “disease associated with increased and/or aberrant IgA” refers to a disease in which there is: (1) increased IgA expression; (2) aberrantly glycosylated IgA as compared to wild type IgA; (3) accumulation of IgA or immune complexes comprising the same; and/or (4) presence of autoantibodies that specifically bind to gd-IgA1 or immune complexes comprising the same. In some embodiments, IgA comprises IgA1 or gd-IgA1. In some embodiments, increased or altered IgA expression includes increased or aberrant expression of IgA1. In some embodiments, increased or altered IgA expression includes increased or aberrant expression of gd-IgA1. In some embodiments, aberrantly glycosylated IgA includes IgA1 having reduced and/or altered glycosylation at a hinge region as compared glycosylation of a reference IgA1 (e.g., wildtype IgA1). In some embodiments, increased or aberrant glycosylation comprises IgA1 glycosylation comprising fewer galactose moieties as compared to IgA1 glycosylation in a healthy individual or a subject who is not at risk of having IgA nephropathy. In some embodiments, reduced glycosylation comprises reduced O-linked glycosylation. In some embodiments, a disease with increased and/or aberrant IgA is characterized in that immune deposits comprising immune complexes (e.g., as described herein) are observed in one or more tissues (e.g., renal tissue) or organs (e.g., kidney). In some embodiments, immune deposits can accumulate in glomeruli. In some embodiments, immune deposits activate mesangial cells. In some embodiments, immune deposits induce renal injury. In some embodiments, a disease with increased and/or aberrant IgA comprises IgA nephropathy (e.g., primary IgA nephropathy or secondary IgA nephropathy). In some embodiments, a disease with increased and/or aberrant IgA comprises dermatitis herpetiformis. In some embodiments, a disease with increased and/or aberrant IgA comprises Henoch-Schoenlein purpura.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system, for example to achieve delivery of an agent that is, or is included in or otherwise delivered by, the composition. Those of ordinary skill in the art will be aware of a variety of routes that may, in appropriate circumstances, be utilized for administration to a subject, for example an animal or a human. In some embodiments, an animal is a domestic animal, such as a companion animal, e.g., a dog or a cat; in some embodiments, an animal is an animal used in agriculture (e.g., farming [e.g., a cow, a sheep or a horse]) or for recreation. For example, in some embodiments, administration may be systemic or local. Those skilled in the art will be aware of appropriate administration routes for use with particular therapies described herein, for example which include bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may be by injection (e.g., intramuscular, intravenous, or subcutaneous injection). In some embodiments, injection may involve bolus injection, drip, perfusion, or infusion. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, an antibody agent can be formulated for oral delivery. For example, one with skill in the art will understand that an antibody agent disclosed herein can be formulated for oral delivery using technologies developed by Oramed (https://www.oramed.com/) or Premas (https://www.premasbiotech.con).

Adult: As used herein, the term “adult” refers to a human eighteen years of age or older. In some embodiments, a human adult has a weight within the range of about 90 pounds to about 250 pounds.

Affinity: As is known in the art, “affinity” is a measure of the tightness with which two or more binding partners associate with one another. Those skilled in the art are aware of a variety of assays that can be used to assess affinity, and will furthermore be aware of appropriate controls for such assays. In some embodiments of a glycoengineered polypeptide disclosed herein, a first moiety comprising one or more peptides that specifically bind to a target autoantibody has a high affinity to an autoantigen. In some embodiments, a high affinity is an affinity of about 100-1000 pM. In some embodiments, affinity is assessed in a quantitative assay. In some embodiments, affinity is assessed over a plurality of concentrations (e.g., of one binding partner at a time). In some embodiments, affinity is assessed in the presence of one or more potential competitor entities (e.g., that might be present in a relevant—e.g., physiological—setting). In some embodiments, affinity is assessed relative to a reference (e.g., that has a known affinity above a particular threshold [a “positive control” reference] or that has a known affinity below a particular threshold [a “negative control” reference” ]. In some embodiments, affinity may be assessed relative to a contemporaneous reference; in some embodiments, affinity may be assessed relative to a historical reference. Typically, when affinity is assessed relative to a reference, it is assessed under comparable conditions.

Avidity: As is known in the art, “avidity” is a measure of the accumulated strength of multiple non-covalent interactions between two or more binding partners in a complex. Those skilled in the art are aware of a variety of assays that can be used to assess avidity, and will furthermore be aware of appropriate controls for such assays. In some embodiments, avidity can be determined by (1) a binding affinity of two or more binding partners in a complex; (2) valency of each of the binding partners in a complex; and/or (3) structural arrangements of two or more binding partners in a complex. In some embodiments, the avidity of binding between two or more binding partners is more than a sum of each binding affinity between the two or more binding partners. In some embodiments, avidity is also referred to as apparent affinity or functional affinity. In some embodiments of a glycoengineered polypeptide disclosed herein, a second moiety comprising one or more glycans which can bind to a receptor (e.g., an endocytic receptor) contributes to binding avidity of the glycoengineered polypeptide. In some embodiments, an endocytic receptor is ASGPR or a fragment or variant thereof. In some embodiments, avidity is assessed in a quantitative assay. In some embodiments, avidity is assessed over a plurality of concentrations. In some embodiments, avidity is assessed in the presence of one or more potential competitor entities (e.g., that might be present in a relevant—e.g., physiological—setting). In some embodiments, avidity may be assessed relative to a contemporaneous reference; in some embodiments, avidity may be assessed relative to a historical reference. Typically, when avidity is assessed relative to a reference, it is assessed under comparable conditions.

Agent: As used herein, the term “agent”, may refer to a physical entity or phenomenon. In some embodiments, an agent may be characterized by a particular feature and/or effect. In some embodiments, an agent may be a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Amino acid: in its broadest sense, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Animal: as used herein refers to a member of the animal kingdom. In some embodiments, “animal” refers to humans; unless otherwise specified, in many embodiments, a human may be of either gender and/or at any stage of development. In some embodiments, “animal” refers to non-human animals; unless otherwise specified, in many embodiments, a non-human animal may be of any gender and/or at any stage of development. In certain embodiments, a non-human animal is a mammal (e.g., a rodent, a mouse, a rat, a rabbit, a monkey, a dog, a cat, a sheep, cattle, a primate, and/or a pig). In some embodiments, an animal may be, for example, a mammals, a bird, a reptile, an amphibian, a fish, an insect, a worm, etc. In some embodiments, an animal may be a transgenic animal, genetically engineered animal, and/or a clone.

Antibody: As used herein, the term “antibody” refers to a polypeptide that includes canonical immunoglobulin sequence elements sufficient to confer specific binding to a particular target antigen. In the case of an autoimmune disease, the antigen to which a pathogenic autoantibody binds is also referred to as an “autoantigen.” As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. In some embodiments, antibodies produced and/or utilized in accordance with the present disclosure include one or more modifications on an Fc domain, e.g., an effector null mutation, e.g., a LALA, LAGA, FEGG, AAGG, or AAGA mutation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of dog, cat, mouse, rabbit, primate, or human antibodies. In some embodiments, antibody sequence elements are human, humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., VHH [e.g., a camelid VHH] or NAR) alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′)2, Fab′, di-sdFv, trifunctional antibodies, diabodies, and minibodies. etc. In some embodiments, relevant formats may be or include: Adnectins®; Affibodies®; Affilins®; Anticalins®; Avimers®; BiTE®s; cameloid antibodies; Centyrins®; ankyrin repeat proteins or DARPINs®; dual-affinity re-targeting (DART) agents; Fynomers®; shark single domain antibodies such as IgNAR; immune mobilixing monoclonal T cell receptors against cancer (ImmTACs); KALBITOR®s; MicroProteins; Nanobodies® minibodies; masked antibodies (e.g., Probodies®); Small Modular ImmunoPharmaceuticals (“SMIPs™”); single chain or Tandem diabodies (TandAb®); TCR-like antibodies; Trans-Bodies®; TrimerX®; VHHs (e.g., camelid VHHs and/or bivalent VHHs). In some embodiments, an antibody may lack a covalent modification (e.g., attachment of a glycan) that it would have if produced naturally. In some embodiments, an antibody format is or comprises a VHH, e.g., a camelid VHH. In some embodiments, a VHH is a multivalent VHH, e.g., a bivalent VHH. In some embodiments, an antibody comprises a single domain antibody, e.g., comprising one or more additional domains such as an Fc, a half-Fc (e.g., comprising an interchain cysteine mutant), an albumin domain, or combinations thereof. In some embodiments, an antibody comprises a single chain Fv, e.g., comprising one or more additional domains such as an Fc, a half-Fc (e.g., comprising an interchain cysteine mutant), an albumin domain, or combinations thereof. In some embodiments, an antibody comprises a polypeptide-Fc fusion. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Antibody fragment: As used herein, an “antibody fragment” refers to a portion of an antibody or antibody agent as described herein, and typically refers to a portion that includes an antigen-binding portion or variable region thereof. An antibody fragment may be produced by any means. For example, in some embodiments, an antibody fragment may be enzymatically or chemically produced by fragmentation of an intact antibody or antibody agent. Alternatively, in some embodiments, an antibody fragment may be recombinantly produced (i.e., by expression of an engineered nucleic acid sequence. In some embodiments, an antibody fragment may be wholly or partially synthetically produced. In some embodiments, an antibody fragment (particularly an antigen-binding antibody fragment) may have a length of at least about 50, 60, 70, 80, 90, 100, 110, 120, 130, 140, 150, 160, 170, 180, 190 amino acids or more, in some embodiments at least about 200 amino acids.

Antigen: The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an elicits a cellular response (e.g., involving T-cells whose receptors specifically interact with the antigen). In some embodiments, and antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Binding: Those skilled in the art will appreciate that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

CDR: as used herein, refers to a complementarity determining region within an antibody variable region. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. Certain systems have been established in the art for defining CDR boundaries (e.g., Kabat, Chothia, etc.); those skilled in the art appreciate the differences between and among these systems and are capable of understanding CDR boundaries to the extent required to understand and to practice the claimed subject matter.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Conjugate: The term “conjugate” as used herein refers to linking of one moiety to another moiety by in vitro methods (e.g., chemical synthesis) or in vivo (e.g., in a cell). In some embodiments, a moiety comprising one or more glycans (e.g., a second moiety) is conjugated to a different moiety, for example, at one or more glycosylation sites in vivo in a cell. In some embodiments, a moiety comprising one or more glycans (e.g., a second moiety) is conjugated to a different moiety, for example, at one or more glycosylation sites by chemical conjugation.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, alpha-helix character, alpha-sheet character, coiled-coil character, random coil character, etc.), and/or by a particular functional feature (e.g., binding activity, enzymatic activity, folding activity, signaling activity, etc.).

Epitope: as used herein, includes any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Functional: As used herein, a “functional” biological molecule is a biological molecule in a form in which it exhibits a property and/or activity by which it is characterized.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the fragment.

Homology: As used herein, the term “homology” refers to the overall relatedness between polymeric molecules, e.g., between polypeptide molecules. In some embodiments, polymeric molecules such as antibodies are considered to be “homologous” to one another if their sequences are at least 80%, 85%, 90%, 95%, or 99% identical. In some embodiments, polymeric molecules are considered to be “homologous” to one another if their sequences are at least 80%, 85%, 90%, 95%, or 99% similar.

Human: In some embodiments, a human is an embryo, a fetus, an infant, a child, a teenager, an adult, or a senior citizen.

Humanized: as is known in the art, the term “humanized” is commonly used to refer to antibodies (or antibody components) whose amino acid sequence includes V_{H} and V_{L} region sequences from a reference antibody raised in a non-human species (e.g., a mouse), but also includes modifications in those sequences relative to the reference antibody intended to render them more “human-like”, i.e., more similar to human germline variable sequences. In some embodiments, a “humanized” antibody (or antibody component) is one that immunospecifically binds to an antigen of interest and that has a framework (FR) region having substantially the amino acid sequence as that of a human antibody, and a complementary determining region (CDR) having substantially the amino acid sequence as that of a non-human antibody. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor immunoglobulin) and all or substantially all of the framework regions are those of a human immunoglobulin consensus sequence. In some embodiments, a humanized antibody also comprises at least a portion of an immunoglobulin constant region (Fc), typically that of a human immunoglobulin constant region. In some embodiments, a humanized antibody contains both the light chain as well as at least the variable domain of a heavy chain. The antibody also may include a C_{H}1, hinge, C_{H}2, C_{H}3, and, optionally, a C_{H}4 region of a heavy chain constant region. In some embodiments, a humanized antibody only contains a humanized V_{L} region. In some embodiments, a humanized antibody only contains a humanized V_{H} region. In some certain embodiments, a humanized antibody contains humanized V_{H} and V_{L} regions.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

“Improve,” “increase”, “inhibit” or “reduce”: As used herein, the terms “improve”, “increase”, “inhibit’, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Peptide: The term “peptide” as used herein refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in a particular form (e.g., in a solid form or a liquid form), and/or may be specifically adapted for, for example: oral administration (for example, as a drenche [aqueous or non-aqueous solutions or suspensions], tablet, capsule, bolus, powder, granule, paste, etc, which may be formulated specifically for example for buccal, sublingual, or systemic absorption); parenteral administration (for example, by subcutaneous, intramuscular, intravenous or epidural injection as, for example, a sterile solution or suspension, or sustained-release formulation, etc); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.

Polypeptide: As used herein refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Specific: The term “specific”, when used herein with reference to an agent having an activity, is understood by those skilled in the art to mean that the agent discriminates between potential target entities or states. For example, an in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

Specificity: As is known in the art, “specificity” is a measure of the ability of a particular ligand to distinguish its binding partner from other potential binding partners.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial identity: as used herein refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215(3): 403-410, 1990; Altschul et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having not more than a small number (e.g., 3, 2, or 1) an amino acid sequence changes relative to that of a reference CDR. In some embodiments, a CDR that is substantially identical to a reference CDR differs from that reference CDR by one or more amino acid changes at the end of the reference CDR; in some such embodiments, the relevant CDR is identical to the reference CDR other than at one or both ends. As is known in the art, CDR elements typically have a length within a range of a few amino acids (e.g., 3, 4, 5, 6, or 7) to about 20 or 30 amino acids (see, for example, Collis et al. J. Mol. Biol. 325:337, 2003, incorporated herein by reference); thus, in some embodiments, a CDR may be considered to be substantially identical to a reference CDR when it shares at least about 80% (or less for a shorter CDR), at least about 85%, at least about 90%, at least about 95%, at least about 98%, at least about 99%, or at least about 100% identity with the reference CDR.

Substantial sequence homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues will appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains Substitution of one amino acid for another of the same type may often be considered a “homologous” substitution. Typical amino acid categorizations are summarized below:

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology. In some embodiments, two sequences are considered to be substantially homologous if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are homologous over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues.

Treat: As used herein, the term “treat,” “treatment,” or “treating” is used to refer to one or more of partial or complete alleviation, amelioration, relief, inhibition, prevention, delay of onset of, reduction in severity of and/or reduction in frequency (e.g., incidence) of one or more symptoms or features of a disease, disorder, and/or condition. In some embodiments, treatment may be prophylactic; for example may be administered to a subject who does not exhibit signs of a disease, disorder, and/or condition. In some embodiments, treatment may be administered to a subject who exhibits early signs of the disease, disorder, and/or condition, and may, for example, decrease risk of developing pathology associated with the disease, disorder, and/or condition and/or delay onset and/or decrease rate of development or worsening of one or more features of a disease, disorder and/or condition.

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors, e.g., that are statistically correlated with increased risk of development of the relevant disease, disorder, and/or condition. Thus, in some embodiments, treatment may be prophylactic; in some embodiments, treatment may be therapeutic.

Variant: The term “variant”, as used herein, refers to a molecule or entity (e.g., that are or comprise a nucleic acid, protein, or small molecule) that shows significant structural identity with a reference molecule or entity but differs structurally from the reference molecule or entity, e.g., in the presence or absence or in the level of one or more chemical moieties as compared to the reference molecule or entity. In some embodiments, a variant also differs functionally from its reference molecule or entity. In many embodiments, whether a particular molecule or entity is properly considered to be a “variant” of a reference is based on its degree of structural identity with the reference molecule. As will be appreciated by those skilled in the art, a biological or chemical reference molecule in typically characterized by certain characteristic structural elements. A variant, by definition, is a distinct molecule or entity that shares one or more such characteristic structural elements but differs in at least one aspect from the reference molecule or entity. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

### DETAILED DESCRIPTION OF CERTAIN EMBODIMENTS

Disclosed herein are glycoengineered polypeptides and compositions comprising the same having the ability to degrade one or more target antibodies (e.g., an IgA1 or an immune complex comprising the same, a gd-IgA1 or an immune complex comprising the same, or an anti-gd-IgA1 autoantibody or an immune complex comprising the same) by binding to a target antibody with a first moiety and binding to an endocytic receptor with a second moiety comprising one or more glycans, thus targeting the target antibody for degradation. As exemplified herein, a glycoengineered polypeptide is engineered by introduction of glycosylation sites on a glycoengineered polypeptide, resulting in an engineered glycosylation profile that mediates endocytic receptor degradation of the glycoengineered polypeptides and the target antibody to which it binds.

By customizing the N-glycosylation, a glycoengineered polypeptide described herein: 1) has homogeneous glycosylation; 2) can degrade large targets such as immune complexes; 3) has a defined ligand-to-antibody ratio; 4) has defined glycosylation sites; 6) can activate more diverse and powerful degradation receptors; and/or 6) can engage in protein degradation in a highly optimized manner. A glycoengineered polypeptide may be employed as a novel therapeutic to treat autoimmune diseases, e.g., a disease associated increased and/or aberrant IgA, e.g., IgA nephropathy.

Diseases Associated with Increased and/or Aberrant IgA

Immunoglobin A (IgA) is the major class of immunoglobulin found in human mucosal secretions. IgA is a polymeric antibody, typically containing two copies of IgA that are assembled with one joining-chain to form a dimeric IgA. The dimeric IgA immunoglobulin protein reaches the fluids of the gastrointestinal and respiratory tract by binding to another polypeptide chain, termed the “polymeric immunoglobulin receptor”, which is produced by mucosal epithelial cells. Once dimeric IgA antibodies bind to this receptor, they are transported by an endocytic transport pathway to the apical surface of the epithelial cell and released into the mucosal fluid space as secretory IgA (sIgA) (Selvskandan et al. Frontiers in Immunology, 2020 and Boyd et al. Kidney International, 2012).

IgA includes two isotypes: IgA1 and IgA2. The main differences between the isotypes IgA1 and IgA2 lie in the hinge region of the heavy polypeptide chain; a 13-amino acid deletion characterizes the IgA2 hinge region (Yamasaki K et al. Monoclon Antib Immunodiagn Immunother, 2018). IgA1 and IgA2 are also found in human blood plasma and serum.

Those skilled in the art, reading the present disclosure, will appreciate that glycoengineered polypeptides disclosed herein (and/or nucleic acid(s) encoding such and/or compositions that comprise and/or deliver either), may be useful for treating a disease associated with increased and/or aberrant IgA in a subject. In some embodiments, a disease associated with increased and/or aberrant IgA has or is characterized as having increased levels of IgA (e.g., circulating IgA1 and/or circulating gd-IgA1) and/or immune complexes comprising the same as compared to a healthy subject who does not have, or does not have the risk of developing a disease associated with increased and/or aberrant IgA. In some embodiments, a disease associated with increased and/or aberrant IgA has or is characterized as having galactose deficient gd-IgA1 as described herein and/or immune complexes comprising the same. In some embodiments, a disease associated with increased and/or aberrant IgA has or is characterized as having anti-gd-IgA1 autoantibodies or immune complexes comprising the same.

Immunoglobulin A1 (IgA1) deposition in human tissues and organs is a characteristic of several human diseases, including IgA nephropathy (IgAN), dermatitis herpetiformis (DH), and Henoch-Schoenlein purpura (HS) ((Hall, R P & T. J. Lawley, J. Immunol. (1985) 135(3): 1760-5), Clarindo M V et al, Bras Dermatol, 2014 and Xu L et al. Front Immunology, 2022). IgA1 deposition can be associated with a variety of clinical manifestations such as renal failure, skin blistering, rash, arthritis, gastrointestinal bleeding and abdominal pain.

In some embodiments, a disease associated with increased and/or aberrant IgA has or is characterized as having IgA deposits (e.g., IgA1 deposits, gd-IgA1 deposits and/or anti-gd-IgA1 deposits). Without wishing to be bound by any particular theory, it is proposed that IgA deposits may be due to accumulation of immune complexes described herein.

Available treatment options for patients that present with abnormal IgA1 deposition can include administration of corticosteroids that have immunosuppressive and anti-inflammatory properties, dietary fish oil supplements that reduce renal inflammation, and angiotensin converting enzyme inhibitors that reduce the risk of progressive renal disease and renal failure. However, such treatments do not reduce levels of IgA1, gd-IgA1 and/or anti-gd-IgA1, and do not directly act on and/or remove IgA1, gd-IgA1 and/or anti-gd-IgA1 deposits in tissue or organs.

A disease associated with increased and/or aberrant IgA can include IgA nephropathy, dermatitis herpetiformis (DH), or Henoch-Schoenlein purpura (HS).

### IgA Nephropathy

IgAN is a disease of the kidney. The disease is considered to be an immune-complex-mediated glomerulonephritis, which is characterized by deposition of IgA either alone or with other immunoglobulins (e.g., IgG and/or IgM) and/or with complement components, in the glomerular mesangium. (Wyatt R J and Julian B A, (2013) NEJM 368:25). Nephropathy results and is defined by proliferative changes in the glomerular mesangial cells. IgAN is one of the most common types of chronic glomerulonephritis and a frequent cause of end-stage renal disease.

Without wishing to be bound by any particular theory, it is proposed that IgAN may develop by the following four steps. The first step is the appearance in the circulation of increased levels of aberrantly O-galactosylated IgA1 (e.g., galactose deficient IgA1 [gd-IgA1]). This IgA1 may also exhibit reduced O-linked sialylation and a reduction in N-acetygalactosamine (GalNAc) residues at the hinge region of IgA1. The second step is the generation of IgG and/or IgA autoantibodies directed against the aberrantly O-galactosylated hinge region of gd-IgA1, with the third step being the formation of anti-gd-IgA1:gd-IgA1 immune complexes. The fourth step is the variable development of inflammatory and fibrotic processes in the kidney, triggered by the deposition of anti-gd-IgA1:gd-IgA1 immune complexes in the mesangium (Selvskandan et al. Frontiers in Immunology, 2020).

In some embodiments, anti-gd-IgA1 is an IgG anti-gd-IgA1.

In some embodiments, anti-gd-IgA1 is an IgM anti-gd-IgA1.

In some embodiments, anti-gd-IgA1 is an IgE anti-gd-IgA1.

In some embodiments, anti-gd-IgA1 is an IgD anti-gd-IgA1.

In some embodiments, IgA deposits occur by accumulation of IgA1 immune complexes (e.g., gd-IgA1 immune complexes and/or IgA1 immune complexes having a normal O-glycosylation). In some embodiments, IgA deposits occur by accumulation of gd-IgA1 immune complexes. In some embodiments, IgA deposits occur by accumulation of IgA1 immune complexes. In some embodiments, IgA deposits occur by accumulation of anti-gd-IgA1 immune complexes, e.g., along with the gd-IgA1 antigen (Selvskandan, H. et al. Frontiers in Immunology, 2020). In some embodiments, an immune complex comprising IgA1, gd-IgA1, and/or anti-gd-IgA1 comprises an antigen recognized by the antibody, one or more components of a complement system, one or more additional immunoglobulins, or combinations thereof.

In some embodiments, disease associated with increased and/or aberrant IgA is IgA nephropathy (IgAN). In some embodiments, the disclosure provides a method for treating IgAN by delivering to a patient in need of such treatment a glycoengineered polypeptide or a composition comprising the same, e.g., as disclosed herein.

Current treatments for IgAN include a blood pressure medication, a proteinuria management therapy, a renal protectant (e.g., a SGLT2 inhibitor), glucocorticoids. In some embodiments, one or more additional treatments (e.g., one or more current treatments listed above) may be administered prior to, substantially simultaneously with, or subsequent to one or more provided glycoengineered polypeptides.

### Dermatitis Herpetiformis (DH)

In some embodiments, a disease associated with increased and/or aberrant IgA is dermatitis herpetiformis (DH). In some embodiments, the disclosure provides a method for treating dermatitis herpetiformis (DH) by delivering to a patient in need of such treatment a glycoengineered polypeptide or a composition comprising the same, e.g., as disclosed herein.

Dermatitis herpetiformis is characterized by a chronic blistering skin disease associated with deposits of IgA1 at the dermal-epidermal junction (Hall, R P & T. J. Lawley, J. Immunol. (1985) 135(3): 1760-5). DH patients have granular IgA1 deposits and often have an associated gluten-sensitive enteropathy (GSE).

Current treatments for Dermatitis herpetiformis include oral antibiotics such as daspone. In some embodiments, one or more additional treatments (e.g., one or more current treatments listed above) may be administered prior to, substantially simultaneously with, or subsequent to one or more provided glycoengineered polypeptides.

### Henoch-Schoenlein Purpura (HS)

In some embodiments, a disease associated with increased and/or aberrant IgA is Henoch-Schoenlein purpura (HS). In some embodiments, the disclosure provides a method for treating Henoch-Schoenlein purpura (HS) by delivering to a patient in need of such treatment a glycoengineered polypeptide or a composition comprising the same, e.g., as disclosed herein.

Henoch-Schoenlein purpura is a skin, blood vessel and kidney disease. HSP is characterized by deposition of IgA1 containing immune complexes in tissue. The disease is often diagnosed by observing evidence of IgA1 deposition in the skin tissue or kidney via immunofluorescence microscopy. The clinical manifestations typically include rash; arthralgias; abdominal pain; and renal disease.

Current treatments for Henoch-Schoenlein purpura include corticosteriods. In some embodiments, one or more additional treatments (e.g., one or more current treatments listed above) may be administered prior to, substantially simultaneously with, or subsequent to one or more provided glycoengineered polypeptides.

### Target Antibodies (I-A1, Gd-IgA1 and Anti-Gd-IgA1)

IgAN, which is the most common primary glomerulonephritis is typically diagnosed from pathological assessment of renal biopsies having IgA-containing immune deposits (Knoppova et al., Frontiers in Immunology (2016) volume 7, article 117). Immune deposits found in IgAN comprise IgA1 including gd-IgA1, complement components and/or other immunoglobulins such as anti-gd-IgA1.

Disclosed herein, among other things, is the finding that glycoengineered polypeptides comprising a first moiety that specifically binds to a target antibody can be used to treat and/or prevent a disease associated with increased and/or aberrant IgA, or ameliorate one or more symptoms associated with increased and/or aberrant IgA.

In some embodiments, a target antibody disclosed herein comprises an IgA1, or a fragment thereof, or an immune complex comprising the same.

In some embodiments, a target antibody disclosed herein comprises a gd-IgA1, or a fragment thereof or an immune complex comprising the same.

In some embodiments, a target antibody disclosed herein comprises an anti-gd-IgA1 autoantibody or a fragment thereof or an immune complex comprising the same. In some embodiments, an anti-gd-IgA1 autoantibody is an IgG. In some embodiments, an anti-gd-IgA1 autoantibody is an IgM. In some embodiments, an anti-gd-IgA1 autoantibody is an IgE. In some embodiments, an anti-gd-IgA1 autoantibody is an IgD.

In some embodiments, a target antibody disclosed herein comprises an immune complex comprising a target antibody. In some embodiments, an immune complex disclosed herein comprises one or more antibodies, an antigen recognized by one or more antibodies, and/or one or more components of a complement system. In some embodiments, a complement component comprises: C3, C5b, C6, C7, C8, and/or C9, or fragments of any complement component or combinations thereof. In some embodiments, an immune complex comprises C3 or fragments of C3. In some embodiments, C3 fragments comprises iC3b, C3c, C3dg, or combinations thereof. In some embodiments, an immune complex comprises or more antibodies chosen from: an IgG, an IgA, an IgM, an IgD, an IgE, or fragments or combinations thereof.

### Glycoengineered Polypeptides

Disclosed herein are glycoengineered polypeptides comprising: (a) a first moiety comprising one or more peptides that specifically bind to a target antibody or a fragment or a complex thereof; and (b) a second moiety comprising one or more glycans conjugated to the first moiety at one or more glycosylation sites.

In some embodiments, a target antibody is an IgA1 antibody or a fragment or a complex thereof. In some embodiments, a first moiety of a glycoengineered polypeptide comprises one or more peptides that specifically bind to an IgA1 antibody or a fragment or a complex thereof. In some embodiments, the one or more peptides that specifically bind to an IgA1 antibody or a fragment thereof or a complex thereof comprises a CD89 polypeptide, or a fragment or a variant thereof. In some embodiments, the one or more peptides that specifically bind to an IgA1 antibody or a fragment thereof or a complex thereof comprises soluble CD89.

In some embodiments, a target antibody is a gd-IgA1 antibody or a fragment or a complex thereof. In some embodiments, a first moiety of a glycoengineered polypeptide comprises one or more peptides that specifically bind to a gd-IgA1 antibody or a fragment or a complex thereof.

In some embodiments, a target antibody is an anti-gd-IgA1 autoantibody or a fragment or a complex thereof. In some embodiments, a first moiety of a glycoengineered polypeptide comprises one or more peptides that specifically bind to an anti-gd-IgA1 autoantibody or a fragment or a complex thereof. In some embodiments, the one or more peptides comprises an epitope recognized by a target antibody (e.g., anti gd-IgA1 autoantibody) or a fragment thereof (e.g., an antigen binding fragment of an anti gd-IgA1 autoantibody). In some embodiments, an epitope comprises a fragment of a gd-IgA1, e.g., a hinge region as described herein. In some embodiments, the one or more peptides specifically binds to one or more idiotopes of an anti-gd-IgA1 autoantibody. In some embodiments, the one or more peptides is an anti-idiotypic antibody that specifically binds to an anti-gd-IgA1 autoantibody.

In some embodiments, a glycoengineered polypeptide is capable of binding to an IgA1 antibody or a fragment or a complex thereof; a gd-IgA1 antibody or a fragment or a complex thereof; and/or an anti-gd-IgA1 autoantibody or a fragment or a complex thereof, or combinations thereof.

In some embodiments, a glycoengineered polypeptide is capable of binding to one or more target antibodies in addition to an IgA1 antibody or a fragment or a complex thereof; a gd-IgA1 antibody or a fragment or a complex thereof; and/or an anti-gd-IgA1 autoantibody or a fragment or a complex thereof, or combinations thereof.

In some embodiments, a glycoengineered polypeptide disclosed herein comprises a first moiety, a second moiety and one or more additional elements. In some embodiments, a glycoengineered polypeptide comprises: an N-glycosylation site, a linker, a spacer, a signal peptide, a tag, a half-life extender or a combination thereof.

In some embodiments, a glycoengineered polypeptide comprises one or more N-glycosylation sites in a first moiety. In some embodiments, a first moiety comprises one or more N-glycosylation sites that are naturally occurring and/or one or more N-glycosylation sites that are engineered into a first moiety. In some embodiments, an engineered N-glycosylation site (also referred to herein as a glycosite) is or comprises the sequence of GGGGANSTAPAPAPA (SEQ ID NO: xx).

In some embodiments, a glycoengineered polypeptide comprises a linker. In some embodiments, a linker comprises a Gly-Ser linker, or an EAAAK linker. In some embodiments, a linker comprises a (Gly-Gly-Gly-Gly-Ser)n linker, wherein n is an integer between 0 to 20.

In some embodiments, a glycoengineered polypeptide comprises a spacer. In some embodiments, a spacer comprises one or more nucleotides which separates a first nucleic acid sequence from a subsequent nucleic acid sequence. In some embodiments, a spacer comprises a nucleic acid sequence which encodes one or more peptides that separates a first encoded polypeptide sequence from a subsequent encoded polypeptide sequence.

In some embodiments, a glycoengineered polypeptide comprises a signal peptide, e.g., as disclosed herein. In some embodiments, a signal peptide is a native signal peptide. In some embodiments, a signal peptide is not a native signal peptide.

In some embodiments, a signal peptide is derived from a Leishmania species. In certain embodiments, a signal peptide is derived from Leishmania tarentolae. In certain embodiments, a signal peptide is derived from Leishmania major.

In certain embodiments, the signal peptide is an invertase signal peptide derived from Leishmania tarentolae.

In certain embodiments, the signal peptide is an alkaline phosphatase signal peptide derived from Leishmania major.

In certain embodiments, a signal peptide comprises an amino acid sequence of SEQ ID NO: 21, or a portion thereof. In certain embodiments, a signal peptide comprises an amino acid sequence of SEQ ID NO: 22, or a portion thereof. In certain embodiments, a signal peptide comprises an amino acid sequence of SEQ ID NO: 42, or a portion thereof. In certain embodiments, a signal peptide comprises an amino acid sequence of SEQ ID NO: 43, or a portion thereof. In certain embodiments, a signal peptide is processed and removed from the glycoengineered polypeptide.

Exemplary signal peptide: SPinv, a modified signal peptide from Leishmania tarentolae invertase, SEQ ID NO: 21: MIASSVRHAVILLLVAVAMMAAVIA.

Exemplary signal peptide: SPinv, the native signal peptide from Leishmania tarentolae invertase, SEQ ID NO: 22: MIASSVRHAVILLLVAVAMMAAAVIA.

Exemplary signal peptide: native signal peptide derived from Leishmania tarentolae invertase Spinv4, SEQ ID NO: 42: MIASSVRHAVILLLVAVAMMGGVIA

Exemplary signal peptide: native signal peptide derived from Leishmania major Alkaline phosphatase (“LmSAP”), SEQ ID NO: 43: MASRLVRVLAAAMLVAAAVS

In some embodiments, a glycoengineered polypeptide comprises a tag. In some embodiments, a tag is a moiety that can be used for purifying and/or identifying a glycoengineered polypeptide disclosed herein. In some embodiments, a tag comprises a His tag, a Myc tag, or a GST tag. In some embodiments, a tag comprises a cleavable tag. In some embodiments, a tag is not a glycotag.

In some embodiments, a tag is a His tag (HHHHHHHHHH; SEQ ID NO: 40).

In some embodiments, a half-life extender comprises albumin or a fragment or a variant thereof. In some embodiments, a half-life extender comprises an Fc mutation.

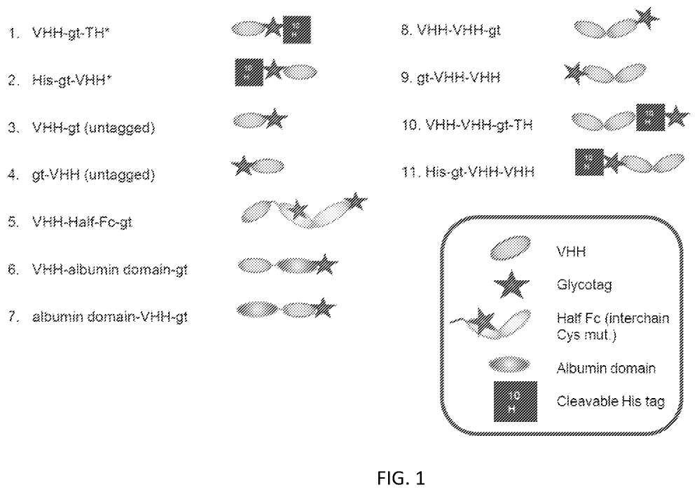

In some embodiments, a glycoengineered polypeptide disclosed herein has a configuration provided in FIG. 1.

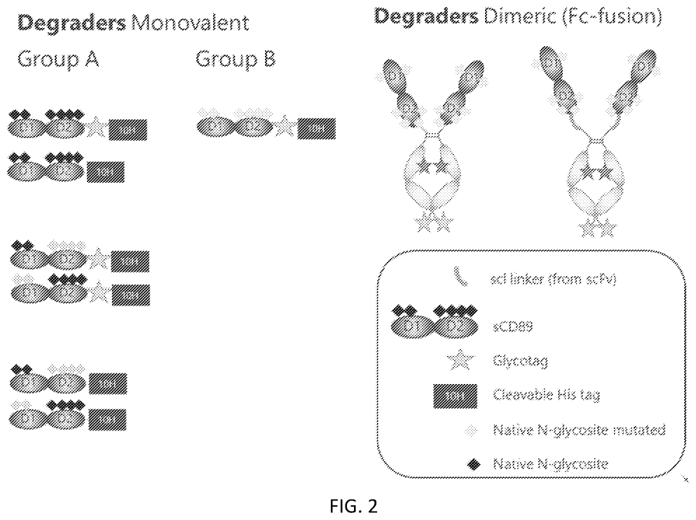

In some embodiments, a glycoengineered polypeptide disclosed herein has a configuration provided in FIG. 2.

### First Moiety

In some embodiments, a glycoengineered polypeptide comprises a first moiety comprising one or more peptides that specifically binds to a target antibody or a fragment thereof or a complex comprising the same.

In some embodiments, a first moiety comprises a peptide that is about 5 amino acids in length to about 500 amino acids in length. In some embodiments, a first moiety comprises a peptide that is about 5 amino acids, about 10 amino acids, about 15 amino acids, about 20 amino acids, about 25 amino acids, about 30 amino acids, about 35 amino acids, about 40 amino acids, about 45 amino acids, about 50 amino acids, about 55 amino acids, about 60 amino acids, about 65 amino acids, about 70 amino acids, about 75 amino acids, about 80 amino acids, about 85 amino acids, about 90 amino acids, about 95 amino acids, about 100 amino acids, about 200 amino acids, about 300 amino acids, about 400 amino acids, about 500 amino acids in length.

In some embodiments, a first moiety comprises a peptide that is at least 5 amino acids, at least 10 amino acids, at least 15 amino acids, at least 20 amino acids, at least 25 amino acids, at least 30 amino acids, at least 35 amino acids, at least 40 amino acids, at least 45 amino acids, at least 50 amino acids, at least 55 amino acids, at least 60 amino acids, at least 65 amino acids, at least 70 amino acids, at least 75 amino acids, at least 80 amino acids, at least 85 amino acids, at least 90 amino acids, at least 95 amino acids, at least 100 amino acids, at least 200 amino acids, at least 300 amino acids, at least 400 amino acids, at least 500 amino acids in length.

In some embodiments, a first moiety comprises one or more peptides that are about 50 amino acids in length to about 5000 amino acids in length in total. In some embodiments, a first moiety comprises one or more peptides that are about 50 amino acids, about 60 amino acids, about 70 amino acids, about 80 amino acids, about 90 amino acids, about 100 amino acids, about 200 amino acids, about 300 amino acids, about 400 amino acids, about 500 amino acids, about 600 amino acids, about 700 amino acids, about 800 amino acids, about 900 amino acids, about 1000 amino acids, about 1500 amino acids, about 2000 amino acids, about 2500 amino acids, about 3000 amino acids, about 3500 amino acids, about 4000 amino acids, about 4500 amino acids, about 5000 amino acids in length in total.

In some embodiments, a first moiety comprises 1, 2, 3, 4, 5, or more peptides that specifically bind to a target antibody.

In some embodiments, one or more peptides of a first moiety that specifically bind to a target antibody are the same, e.g., the one or more peptides have the same sequence. In some embodiments, the one or more peptides having the same sequence are separated by one or more intervening sequences (e.g., spacers, and/or linkers). In some embodiments, the one or more peptides having the same sequence are not separated by one or more intervening sequences.

In some embodiments, one or more peptides of a first moiety that specifically bind to a target antibody are different, e.g., the one or more peptides do not have the same sequence. In some embodiments, the one or more peptides having different sequences are separated by one or more intervening sequences (e.g., spacers, tags and/or linkers). In some embodiments, the one or more peptides different sequences are not separated by one or more intervening sequences (e.g., spacers, tags and/or linkers).

In some embodiments, the one or more peptides having different sequences comprise one or more peptides that specifically bind to the same target antibody. In some embodiments, the one or more peptides bind to different epitopes of a target antibody (e.g., to different domains of the target antibody). In some embodiments, the one or more peptides having different sequences comprise one or more peptides that specifically bind to an IgA1 or a fragment thereof. In some embodiments, the one or more peptides having different sequences comprise one or more peptides that specifically bind to a gd-IgA1 or a fragment thereof. In some embodiments, the one or more peptides having different sequences comprise one or more peptides that specifically bind to an anti-gdIgA1 autoantibody or a fragment thereof.

In some embodiments, the one or more peptides having different sequences comprise one or more peptides that specifically bind to a first a target antibody (e.g., a IgA1 or a fragment thereof), one or more peptides that bind to a second target antibody (e.g., a gd-IgA1 or a fragment thereof) and one or more peptides that bind to a third target antibody (e.g., an anti-gd-IgA1 autoantibody or a fragment thereof). In some embodiments, the one or more peptides having different sequences are separated by one or more intervening sequences (e.g., spacers, tags and/or linkers). In some embodiments, the one or more peptides different sequences are not separated by one or more intervening sequences (e.g., spacers, tags and/or linkers).

In some embodiments, a linker separating one or more peptides of a first moiety comprises a Gly-Ser linker, or an EAAAK linker. In some embodiments, a linker comprises a (Gly-Gly-Gly-Gly-Ser)n linker, wherein n is an integer between 0 to 20.

In some embodiments, a spacer separating one or more peptides of a first moiety comprises 1-10 amino acid residues, or about 10-20 amino acid residues.

In some embodiments, one or more peptides that specifically bind to a target antibody are each conjugated to a second moiety.

In some embodiments, one or more peptides that specifically bind to a target antibody are not each conjugated to the second moiety.

In some embodiments, one or more peptides that specifically bind to a target antibody are conjugated to each other, e.g., are situated on one polypeptide.

In some embodiments, one or more peptides that specifically bind to a target antibody are separated by a protease cleavage site or an IRES. In some embodiments, each of the one or more peptides is expressed as a separate peptide, e.g., translation as a separate peptide from an IRES or after cleavage of a protease cleavage site.

In some embodiments, one or more peptides that specifically bind to a target antibody are not separated by a protease cleavage site or an IRES, e.g., is expressed as a fusion protein.

In some embodiments, one or more peptides of a first moiety that specifically bind to a target antibody comprise an epitope that is recognized by a target antibody. In some embodiments, an epitope is a linear epitope. In some embodiments, an epitope is a conformational epitope.

In some embodiments, an epitope is or comprises a single continuous epitope. In some embodiments, an epitope comprises one or more additional amino acid residues, e.g., on the 5′ end and/or the 3′ end of the epitope.

In some embodiments, an epitope comprises one or more sequences separated by one or more intervening amino acid sequences configured such that the one or more sequences form a single epitope, e.g., spatially form an epitope when expressed and folded into a polypeptide conformation. In some embodiments, an intervening amino acid sequence comprises a linker and/or a spacer. For example, an epitope comprising one or more sequences separated by one or more intervening amino acid sequences has the following structure: Xn-[A1]-Xn-[A2]-Xn, wherein A1 is a first portion of an epitope and A2 is a second portion of an epitope which together form a spatial epitope that is recognized by a target antibody, and X denotes intervening amino acid sequences with n being an integer from 0-20. In some embodiments, an intervening amino acid sequence is a spacer or a linker, e.g., as described herein.

In some embodiments, an epitope that is formed by one or more sequences can be broken up into 3, 4, 5, or more fragments. For example, in such embodiments, the polypeptide may comprise the following structure: Xn-[A1]-Xn-[A2]-Xn-[An]-Xn, wherein A1 is a first portion of an epitope, A2 is a second portion of an epitope, and An is the n-th portion of an epitope which together form a spatial epitope that is recognized by a target antibody, and X denotes intervening amino acid sequences with n being an integer from 0-20. In some embodiments, an intervening amino acid sequence is a spacer or a linker, e.g., as described herein.

In some embodiments, a first moiety comprises a plurality of epitopes, e.g., the same or different epitopes. In some embodiments, a first moiety comprises a plurality of the same epitopes, e.g., epitopes recognized by the same target antibody. In some embodiments, a first moiety comprises a plurality of different epitopes, e.g., epitopes recognized by different target antibodies. In some embodiments, the plurality of epitopes is separated by a linker, IRES or cleavage peptide.

Immunoglobulin A1 (IgA1) and Peptides that Bind to IgA1

In humans, there are two types of IgA subclasses, IgA1 and IgA2. IgA1 has O-glycans attached to Serine or Threonine residues in the hinge region of the heavy chain (Knoppova 2017). The IgA2 hinge region does not have Serine or Threonine residues and IgA2 does not have O-glycans.

A human IgA1 constant region polypeptide sequence is provided herein as SEQ ID NO: 2:

ASPTSPKVFPLSLCSTQPDGNVVIACLVQGFFPQEPLSVTWSESG
QGVTARNFPPSQDASGDLYTTSSQLTLPATQCLAGKSVTCHVKHY
TNPSQDVTVPCPVPSTPPTPSPSTPPTPSPSCCHPRLSLHRPALE
DLLLGSEANLTCTLTGLRDASGVTFTWTPSSGKSAVQGPPERDLC
GCYSVSSVLPGCAEPWNHGKTFTCTAAYPESKTPLTATLSKSGNT
FRPEVHLLPPPSEELALNELVTLTCLARGFSPKDVLVRWLQGSQE
LPREKYLTWASRQEPSQGTTTFAVTSILRVAAEDWKKGDTFSCMV
GHEALPLAFTQKTIDRLAGKPTHVNVSVVMAEVD
GTCY

In some embodiments, a glycoengineered polypeptide comprises a first moiety comprising one or more peptides that specifically bind to IgA1. In some embodiments, the one or more peptides that specifically bind to IgA1 recognize an epitope on IgA1.

In some embodiments, an epitope is a linear epitope. In some embodiments, an epitope is a conformational epitope. In some embodiments, an epitope is or comprises a single continuous epitope. In some embodiments, an epitope comprises one or more additional amino acid residues, e.g., on the 5′ end and/or the 3′ end of the epitope.

In some embodiments, an epitope comprises one or more sequences separated by one or more intervening amino acid sequences configured such that the one or more sequences form a single epitope, e.g., spatially form an epitope when expressed and folded into a polypeptide conformation. In some embodiments, an intervening amino acid sequence comprises a linker and/or a spacer. For example, an epitope comprising one or more sequences separated by one or more intervening amino acid sequences has the following structure: Xn-[A1]-Xn-[A2]-Xn, wherein A1 is a first portion of an epitope and A2 is a second portion of an epitope which together form a spatial epitope that is recognized by a target antibody, and X denotes intervening amino acid sequences with n being an integer from 0-20.

In some embodiments, an epitope that is formed by one or more sequences can be broken up into 3, 4, 5, or more fragments. For example, in such embodiments, the polypeptide may comprise the following structure: Xn-[A1]-Xn-[A2]-Xn-[An]-Xn, wherein A1 is a first portion of an epitope, A2 is a second portion of an epitope, and An is the n-th portion of an epitope which together form a spatial epitope that is recognized by a target antibody, and X denotes intervening amino acid sequences with n being an integer from 0-20.

In some embodiments, a first moiety comprises a plurality of epitopes, e.g., the same or different epitopes. In some embodiments, a first moiety comprises a plurality of the same epitopes, e.g., epitopes recognized by an IgA1 antibody. In some embodiments, a first moiety comprises a plurality of different epitopes, e.g., epitopes recognized by different IgA1 antibodies. In some embodiments, the plurality of epitopes is separated by a linker, IRES or cleavage peptide.

In some embodiments, one or more peptides that specifically bind to an anti-IgA1 antibody comprises an epitope that is not present or not accessible in an IgA1 from a healthy individual or an individual not at risk of developing IgAN.

In some embodiments, a first moiety comprises 1, 2, 3, 4, 5, or more peptides that specifically bind to an IgA1 antibody.

In some embodiments, one or more peptides of a first moiety that specifically bind to an IgA1 antibody are the same, e.g., the one or more peptides have the same sequence. In some embodiments, the one or more peptides having the same sequence are separated by one or more intervening sequences (e.g., spacers and/or linkers). In some embodiments, the one or more peptides having the same sequence are not separated by one or more intervening sequences (e.g., spacers and/or linkers).

In some embodiments, one or more peptides of a first moiety that specifically bind to an IgA1 antibody are different, e.g., the one or more peptides do not have the same sequence. In some embodiments, the one or more peptides having different sequences are separated by one or more intervening sequences (e.g., spacers and/or linkers). In some embodiments, the one or more peptides different sequences are not separated by one or more intervening sequences (e.g., spacers and/or linkers).

In some embodiments, each of the one or more peptides having different sequences specifically bind to an IgA1 antibody or a fragment thereof.

In some embodiments, a linker separating one or more peptides of a first moiety comprises a Gly-Ser linker, or an EAAAK linker. In some embodiments, a linker comprises a (Gly-Gly-Gly-Gly-Ser)n linker, wherein n is an integer between 0 to 20.

In some embodiments, one or more peptides that specifically bind to an IgA1 antibody are each conjugated to a second moiety.

In some embodiments, one or more peptides that specifically bind to an IgA1 antibody are not each conjugated to the second moiety.

In some embodiments, one or more peptides that specifically bind to an IgA1 antibody are conjugated to each other, e.g., are situated on one polypeptide.

In some embodiments, one or more peptides that specifically bind to an IgA1 antibody are separated by a protease cleavage site or an IRES. In some embodiments, each of the one or more peptides is expressed as a separate peptide, e.g., translation as a separate peptide from an IRES or after cleavage of a protease cleavage site.

In some embodiments, one or more peptides that specifically bind to an IgA1 antibody are not separated by a protease cleavage site or an IRES, e.g., is expressed as a fusion protein.

In some embodiments, a glycoengineered polypeptide comprising a first moiety which comprises one or more peptides that specifically binds to an IgA1 antibody or a fragment thereof has a configuration provided in FIG. 1.

In some embodiments, one or more peptides that specifically bind to an anti-IgA1 antibody comprises an antibody agent. In some embodiments, the antibody agent comprises an antigen binding fragment. In some embodiments, the antibody agent comprises a full antibody, a Fab fragment, an scFv, a nanobody, a duobody, a single domain antibody (e.g., a VHH). In some embodiments, the antibody agent comprises a VHH, e.g., a camelid VHH or a bivalent VHH.

### CD89 Polypeptides as IgA1 Binders

CD89, also known as immunoglobulin alpha Fc receptor (FcaR1), is a human myeloid IgA Fc receptor and binds to both IgA1 and IgA2 subclasses of IgA (Morton H C and Brandtzaeg P (2001) Arch Immunol Ther Exp (Warsz) 49(3):217-29.

An exemplary human CD89 polypeptide sequence is provided herein as SEQ ID NO: 5, with bolding indicating the signal sequence (corresponding to UniProt Accession Number P24071):

MDPKQTTLLCLVLCLGQRIQAQEGDFPMPFISAKSSPVIPLDGSV
KIQCQAIREAYLTQLMIIKNSTYREIGRRLKFWNETDPEFVIDHM
DANKAGRYQCQYRIGHYRFRYSDTLELVVTGLYGKPFLSADRGLV
LMPGENISLTCSSAHIPFDRFSLAKEGELSLPQHQSGEHPANFSL
GPVDLNVSGIYRCYGWYNRSPYLWSFPSNALELVVTDSIHQDYTT
QNLIRMAVAGLVLVALLAILVENWHSHTALNKEASADVAEPSWSQ
QMCQPGLTFARTPSVCK
Human CD89 signal peptide:
(SEQ ID NO: 44)
MDPKQTTLLCLVLCLGQRIQA

An exemplary human CD89 polypeptide sequence is provided herein as SEQ ID NO: 9, with bolding indicating the signal peptide of SEQ ID NO: 44:

MDPKQTTLLCLVLCLGQRIQAQEGRYISEHFWCRSLGCNPVNDAS
AQRPGDFPMPFISAKSSPVIPLDGSVKIQCQAIREAYLTQLMIIK
NSTYREIGRRLKFWNETDPEFVIDHMDANKAGRYQCQYRIGHYRF
RYSDTLELVVTDSIHQDYTTQNLIRMAVAGLVLVALLAILVENWH
SHTALNKEASADVAEPSWSQQMCQPGLTFARTPSVCK

An engineered human CD89 polypeptide sequence is provided herein as SEQ ID NO: 38, with bolding indicating the signal peptide of SEQ ID NO: 42:

MIASSVRHAVILLLVAVAMMGGVIAQEGDFPMPFISAKSSPVIPL
DGSVKIQCQAIREAYLTQLMIIKNSTYREIGRRLKFWNETDPEFV
IDHMDANKAGRYQCQYRIGHYRFRYSDTLELVVTGLYGKPFLSAD
RGLVLMPGEQISLTCSSAHIPFDRFSLAKEGELSLPQHQSGEHPA
QFSLGPVDLQVSGIYRCYGWYQRSPYLWSFPSNALELVVT

An exemplary engineered CD89 polypeptide sequence without a signal peptide is provided herein as SEQ ID NO: 39

QEGDFPMPFISAKSSPVIPLDGSVKIQCQAIREAYLTQLMIIKNS
TYREIGRRLKFWNETDPEFVIDHMDANKAGRYQCQYRIGHYRFRY
SDTLELVVTGLYGKPFLSADRGLVLMPGEQISLTCSSAHIPFDRF
SLAKEGELSLPQHQSGEHPAQFSLGPVDLQVSGIYRCYGWYQRSP
YLWSFPSNALELVVT

In some embodiments, one or more peptides in a first moiety that specifically bind to IgA1, or a fragment or a complex thereof comprise a CD89 (FcaR1) polypeptide, or a variant, or a fragment thereof.

In some embodiments, the one or more peptides comprise a soluble form of CD89 of a variant or a fragment thereof. Soluble CD89 is described e.g., in van Zandbergen G et al., (1999) J Immunology 163, pp. 5806-5812; and in van Der Boog P J M et al., (2002) J Immunology 168.3 pp. 1252-1258, the entire contents of each of which are hereby expressly incorporated by reference.

In some embodiments, a glycoengineered polypeptide comprising a first moiety which comprises one or more peptides comprising a CD89 (FcaR1) polypeptide, or a variant, or a fragment thereof has a configuration provided in FIG. 2.

In some embodiments, a CD89 polypeptide comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 5. In some embodiments, a CD89 polypeptide is or comprises SEQ ID NO: 5 or SEQ ID NO: 9. In some embodiments, a CD89 polypeptide is or comprises SEQ ID NO: 5 or SEQ ID NO: 9 without the signal peptide of SEQ ID NO: 44.

In some embodiments, a CD89 polypeptide comprises a sequence having at least 85% identity to SEQ ID NO: 5 without the signal peptide of SEQ ID NO: 44. In some embodiments, a CD89 polypeptide comprises a sequence having at least 85% identity to SEQ ID NO: 9 without the signal peptide of SEQ ID NO: 44.

In some embodiments, a CD89 polypeptide comprising a sequence having at least 85% identity to SEQ ID NO: 5 without the signal peptide of SEQ ID NO: 44, further comprises a different signal peptide, e.g., as disclosed herein.

In some embodiments, a CD89 polypeptide comprising a sequence having at least 85% identity to SEQ ID NO: 9 without the signal peptide of SEQ ID NO: 44, further comprises a different signal peptide, e.g., as disclosed herein.

In some embodiments, a glycoengineered polypeptide comprises a first moiety comprising one or more peptides comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 5 or SEQ ID NO: 9. In some embodiments, a first moiety comprises one or more peptides comprising the sequence of SEQ ID NO: 5 or SEQ ID NO: 9. In some embodiments, a first moiety comprises one or more peptides comprising the sequence of SEQ ID NO: 5 without the signal peptide of SEQ ID NO: 44. In some embodiments, a first moiety comprises one or more peptides comprising the sequence of SEQ ID NO: 9 without the signal peptide of SEQ ID NO: 44.

In some embodiments, a first moiety comprises one or more peptides comprising a sequence having at least 85% identity to SEQ ID NO: 5 without the signal peptide of SEQ ID NO: 44. In some embodiments, a first moiety comprises one or more peptides comprising a sequence having at least 85% identity to SEQ ID NO: 9 without the signal peptide of SEQ ID NO: 44.

In some embodiments, a first moiety comprising one or more peptides comprising a sequence having at least 85% identity to SEQ ID NO: 5 without the signal peptide of SEQ ID NO: 44 further comprises a different signal peptide, e.g., as disclosed herein.

In some embodiments, a first moiety comprising one or more peptides comprising a sequence having at least 85% identity to SEQ ID NO: 9 without the signal peptide of SEQ ID NO: 44 further comprises a different signal peptide, e.g., as disclosed herein.

In some embodiments, a CD89 polypeptide comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 38. In some embodiments, a CD89 polypeptide is or comprises SEQ ID NO: 38. In some embodiments, a CD89 polypeptide is or comprises SEQ ID NO: 38 without the signal peptide of SEQ ID NO: 42.

In some embodiments, a CD89 polypeptide comprises a sequence having at least 85% identity to SEQ ID NO: 38 without the signal peptide of SEQ ID NO: 42.

In some embodiments, a CD89 polypeptide comprising a sequence having at least 85% identity to SEQ ID NO: 38 without the signal peptide of SEQ ID NO: 42, further comprises a different signal peptide, e.g., as disclosed herein.

In some embodiments, a glycoengineered polypeptide comprises a first moiety comprising one or more peptides comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 38. In some embodiments, a first moiety comprises one or more peptides comprising the sequence of SEQ ID NO: 38. In some embodiments, a first moiety comprises one or more peptides comprising the sequence of SEQ ID NO: 38 without the signal peptide of SEQ ID NO: 42.

In some embodiments, a first moiety comprises one or more peptides comprising a sequence having at least 85% identity to SEQ ID NO: 38 without the signal peptide of SEQ ID NO: 42.

In some embodiments, a first moiety comprising one or more peptides comprising a sequence having at least 85% identity to SEQ ID NO: 38 without the signal peptide of SEQ ID NO: 42, further comprises a different signal peptide, e.g., as disclosed herein.

In some embodiments, a CD89 polypeptide comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 39. In some embodiments, a CD89 polypeptide is or comprises SEQ ID NO: 39.

In some embodiments, a CD89 polypeptide comprising an amino acid sequence having at least 85% identity to SEQ ID NO: 39 comprises a signal peptide disclosed herein, e.g., a Leishmania derived signal peptide or a CD89 signal peptide.

In some embodiments, a signal peptide is chosen from SEQ ID NO: 21, SEQ ID NO: 22, SEQ ID NO: 42, SEQ ID NO: 43 or SEQ ID NO: 44.

In some embodiments, a glycoengineered polypeptide comprises a first moiety comprising one or more peptides comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 39. In some embodiments, a first moiety comprises one or more peptides comprising the sequence of SEQ ID NO: 39.

In some embodiments, a first moiety comprises one or more peptides comprising a sequence having at least 85% identity to SEQ ID NO: 39 and a signal peptide, e.g., as disclosed herein. In some embodiments, a signal peptide is chosen from SEQ ID NO: 21, SEQ ID NO: 22, SEQ ID NO: 42, SEQ ID NO: 43 or SEQ ID NO: 44.

In some embodiments, a target antibody is an IgA1 antibody or a fragment thereof. In some embodiments, an IgA1 antibody is characterized in that it binds to a CD89 polypeptide or a variant or fragment thereof.

In some embodiments, a first moiety of a glycoengineered polypeptide disclosed herein comprises one or more peptides that specifically bind to an IgA1 antibody. In some embodiments, one or more peptides that specifically bind to an IgA1 antibody comprises a CD89 polypeptide, or a fragment or a variant thereof.

In some embodiments, one or more peptides that specifically bind to an IgA1 antibody comprises a soluble fragment of a CD89 polypeptide.

In some embodiments, one or more peptides that specifically bind to an IgA1 antibody comprises a fragment of a CD89 polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a CD89 polypeptide of SEQ ID NO: 5 or SEQ ID NO: 9.

In some embodiments, one or more peptides that specifically bind to an IgA1 antibody comprises a fragment of a CD89 polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a CD89 polypeptide of SEQ ID NO: 5 or SEQ ID NO: 9 without the signal peptide.

In some embodiments, one or more peptides that specifically bind to an IgA1 antibody comprises a fragment of a CD89 polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a CD89 polypeptide of SEQ ID NO: 5 or SEQ ID NO: 9.

In some embodiments, one or more peptides that specifically bind to an IgA1 antibody comprises a fragment of a CD89 polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a CD89 polypeptide of SEQ ID NO: 5 or SEQ ID NO: 9 without the signal peptide.

In some embodiments, one or more peptides that specifically bind to an IgA1 antibody comprises a fragment of a CD89 polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a CD89 polypeptide of SEQ ID NO: 38.

In some embodiments, one or more peptides that specifically bind to an IgA1 antibody comprises a fragment of a CD89 polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a CD89 polypeptide of SEQ ID NO: 38 without the signal peptide.

In some embodiments, one or more peptides that specifically bind to an IgA1 antibody comprises a fragment of a CD89 polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a CD89 polypeptide of SEQ ID NO: 38.

In some embodiments, one or more peptides that specifically bind to an IgA1 antibody comprises a fragment of a CD89 polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a CD89 polypeptide of SEQ ID NO: 38 without the signal peptide.

In some embodiments, one or more peptides that specifically bind to an IgA1 antibody comprises a fragment of a CD89 polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a CD89 polypeptide of SEQ ID NO: 39.

In some embodiments, one or more peptides that specifically bind to an IgA1 antibody comprises a fragment of a CD89 polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a CD89 polypeptide of SEQ ID NO: 39.

In some embodiments, a fragment comprises a CD89 fragment that binds to IgA1. In some embodiments, the CD89 fragment that binds to IgA1 is a linear fragment. In some embodiments, the CD89 fragment that binds to IgA1 is a conformational fragment. In some embodiments, the fragment comprises one or more additional amino acid residues, e.g., on the 5′ end and/or the 3′ end of the epitope.

In some embodiments, a CD89 fragment which binds to IgA1 comprises one or more sequences separated by one or more intervening amino acid sequences configured such that the one or more sequences form a conformation that can be bound by IgA1. In some embodiments, an intervening amino acid sequence comprises a linker and/or a spacer. For example, a CD89 fragment which binds to IgA1 comprising one or more sequences separated by one or more intervening amino acid sequences has the following structure: Xn-[A1]-Xn-[A2]-Xn, wherein A1 is a first portion of a CD89 which binds to IgA1 and A2 is a second portion of a CD89 fragment which binds to IgA1 which together form a conformation that can be bound by IgA1, and X denotes intervening amino acid sequences with n being an integer from 0-20.

In some embodiments, a CD89 fragment which binds to IgA1 that is formed by one or more sequences can be broken up into 3, 4, 5, or more fragments. For example, in such embodiments, the polypeptide may comprise the following structure: Xn-[A1]-Xn-[A2]-Xn-[An]-Xn, wherein A1 is a first portion of a CD89 fragment which binds to IgA1, A2 is a second portion of a CD89 fragment which binds to IgA1, and An is the n-th portion of a CD89 fragment which binds to IgA1 which together form a conformation that can be bound by IgA1, and X denotes intervening amino acid sequences with n being an integer from 0-20.

In some embodiments, a first moiety comprises a plurality of CD89 fragments that bind to IgA1, e.g., the same or different CD89 fragments that bind to IgA1. In some embodiments, a first moiety comprises a plurality of the same CD89 fragments that bind to IgA1. In some embodiments, a first moiety comprises a plurality of different CD89 fragments that bind to IgA1. In some embodiments, the plurality of CD89 fragments that bind to IgA1 is separated by a linker, IRES or cleavage peptide.

In some embodiments, one or more peptides that specifically bind to an IgA1 antibody comprises a contiguous chain of amino acids comprising at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, of the amino acid of SEQ ID NO: 5 or SEQ ID NO: 9.

In some embodiments, one or more peptides that specifically bind to an IgA1 antibody comprises a contiguous chain of amino acids comprising at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, of the amino acid of SEQ ID NO: 5 or SEQ ID NO: 9 without the signal peptide.

In some embodiments, one or more peptides that specifically bind to an IgA1 antibody comprises a contiguous chain of amino acids comprising at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, of the amino acid of SEQ ID NO: 38.

In some embodiments, one or more peptides that specifically bind to an IgA1 antibody comprises a contiguous chain of amino acids comprising at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, of the amino acid of SEQ ID NO: 39.

In some embodiments, one or more peptides that specifically bind to an IgA1 antibody comprises a contiguous chain of amino acids comprising at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, of the amino acid of SEQ ID NO: 41.

In some embodiments, one or more peptides that specifically bind to an IgA1 antibody comprises a full length CD89 protein, e.g., as provided in SEQ ID NO: 5 with or without the signal peptide.

In some embodiments, one or more polypeptides that specifically bind to an anti-IgA1 antibody comprises a variant of a CD89 polypeptide. In some embodiments, a variant is an inactive variant as compared to a wild-type CD89 polypeptide.

In some embodiments, a variant comprises a CD89 polypeptide or a fragment thereof having one or more mutations at a glycosylation site. In some embodiments, a mutation is at an Asparagine residue such that a glycosylation site is altered. In some embodiments, a mutation comprises a mutation at Asparagine 141, Asparagine 177, Asparagine 186 and/or Asparagine 198.

In some embodiments, a glycoengineered polypeptide comprises a CD89 polypeptide or a fragment thereof having one or more native glycosylation sites. In some embodiments, a glycoengineered polypeptide comprises a CD89 polypeptide or a fragment thereof having two native glycosylation sites. In some embodiments, a native glycosylation site comprises N65 and/or N79.

In some embodiments, a glycoengineered polypeptide comprises a CD89 polypeptide or a fragment thereof comprising native glycosylation sites: N65 and N79.

In some embodiments, a glycoengineered polypeptide comprises a CD89 polypeptide or a fragment thereof having one or more engineered glycosylation sites. In some embodiments, an engineered glycosylation site is or comprises the sequence of

(SEQ ID NO: 37)
GGGGANSTAPAPAPA.

In some embodiments, a glycoengineered polypeptide comprises a CD89 polypeptide or a fragment thereof having two native glycosylation sites: N65 and N79, and one engineered glycosylation site having the sequence GGGGANSTAPAPAPA (SEQ ID NO: 37).

In some embodiments, a glycoengineered polypeptide further comprises one or more additional elements. In some embodiments, one or more additional elements comprise: (a) a linker, (b) a spacer, (c) a cleavage peptide, e.g., an IRES or a protease cleavage site, (d) a signal peptide, (e) a tag, e.g., a cleavable tag, (f) a half-life extender domain, e.g., an Fc domain or albumin, or (g) any combination of (a)-(f).

In some embodiments, a glycoengineered polypeptide comprises a tag, e.g., a His tag.

In some embodiments, a glycoengineered polypeptide comprises the following sequence (SEQ ID NO: 41).

MIASSVRHAVILLLVAVAMMGGVIAQEGDFPMPFISAKSSPVIPL
DGSVKIQCQAIREAYLTQLMIIKNSTYREIGRRLKFWNETDPEFV
IDHMDANKAGRYQCQYRIGHYRFRYSDTLELVVTGLYGKPFLSAD
RGLVLMPGEQISLTCSSAHIPFDRFSLAKEGELSLPQHQSGEHPA
QFSLGPVDLQVSGIYRCYGWYQRSPYLWSFPSNALELVVTGGGGA
NSTAPAPAPAHHHHHHHHHH

In some embodiments, a glycoengineered polypeptide comprises a sequence with at least 85%, at least 90%, at least 95%, at least 99% identity to SEQ ID NO: 41. In some embodiments, a glycoengineered polypeptide comprises the sequence of SEQ ID NO: 41.

In some embodiments, a first moiety comprises 1, 2, 3, 4, 5, or more peptides that specifically bind to an IgA1 antibody.

In some embodiments, one or more peptides of a first moiety that specifically bind to an IgA1 antibody are the same, e.g., the one or more peptides have the same sequence of a CD89 polypeptide, or a fragment or a variant thereof. In some embodiments, the one or more peptides having the same sequence of a CD89 polypeptide, or a fragment or a variant thereof are separated by one or more intervening sequences (e.g., spacers and/or linkers). In some embodiments, the one or more peptides having the same sequence of a CD89 polypeptide, or a fragment or a variant thereof are not separated by one or more intervening sequences (e.g., spacers and/or linkers).

In some embodiments, one or more peptides of a first moiety that specifically bind to an IgA1 antibody are different, e.g., the one or more peptides do not have the same sequence of a CD89 polypeptide, or a fragment or a variant thereof. In some embodiments, the one or more peptides having different sequences of a CD89 polypeptide, or a fragment or a variant thereof are separated by one or more intervening sequences (e.g., spacers and/or linkers). In some embodiments, the one or more peptides different sequences of a CD89 polypeptide, or a fragment or a variant thereof are not separated by one or more intervening sequences (e.g., spacers and/or linkers).

In some embodiments, each of the one or more peptides having different sequences of a CD89 polypeptide, or a fragment or a variant thereof specifically bind to an IgA1 antibody or a fragment thereof.

In some embodiments, a linker separating one or more peptides of a first moiety comprises a Gly-Ser linker, or an EAAAK linker. In some embodiments, a linker comprises a (Gly-Gly-Gly-Gly-Ser)n linker, wherein n is an integer between 0 to 20.

In some embodiments, one or more peptides that specifically bind to an IgA1 antibody are each conjugated to a second moiety.

In some embodiments, one or more peptides that specifically bind to an IgA1 antibody are not each conjugated to the second moiety.

In some embodiments, one or more peptides that specifically bind to an IgA1 antibody are conjugated to each other, e.g., are situated on one polypeptide.

In some embodiments, one or more peptides that specifically bind to an IgA1 antibody are separated by a protease cleavage site or an IRES. In some embodiments, each of the one or more peptides is expressed as a separate peptide, e.g., translation as a separate peptide from an IRES or after cleavage of a protease cleavage site.

In some embodiments, one or more peptides that specifically bind to an IgA1 antibody are not separated by a protease cleavage site or an IRES, e.g., is expressed as a fusion protein.

Galactose-Deficient IgA1 (Gd-IgA1) and Peptides that Bind to Gd-IgA1

As described herein, IgA1 has a hinge region comprising Serine and Threonine residues that can be glycosylated. IgA1 has a hinge region comprising nine Serine and Threonine amino acid residues, out of which about three to six residues are typically attached to O-glycans (Knoppova 2017). In humans, IgA1 hinge region glycoforms having four and give glycans are most common.

As described in Knoppova 2017, each heavy chain of IgA1 also contains two N-glycans, one in the CH2 domain (Asn263) and the second in the tailpiece portion (Asn459). Additionally, normal human circulatory IgA1 usually has core 1 O-glycans consisting of N-acetylgalactosamine (GalNAc) with β1,3-linked galactose. One or both saccharides can be sialylated, galactose with α2,3-linked and GalNAc with α2,6-linked sialic.

The composition of the O-glycans on normal serum IgA1 is variable with the most common forms including the GalNAc-galactose disaccharide and its mono- and di-sialylated forms. Typically, normal serum IgA1 has little or no galactose-deficient O-glycans, but it has been shown that some terminal or sialylated GalNAc can be found even in healthy individuals (Knoppova 2017).

In IgA nephropathy patients, IgA1 having aberrant O-glycosylation is commonly found. In particular, galactose-deficient IgA1 (gd-IgA1) has been shown to play a role in the formation of immune complexes and glomerular deposition. Several reports suggest that the IgA in the mesangial deposits is mainly of the IgA1 subclass, and is enriched for Gd-IgA1 glycoforms (Knoppova 2017).

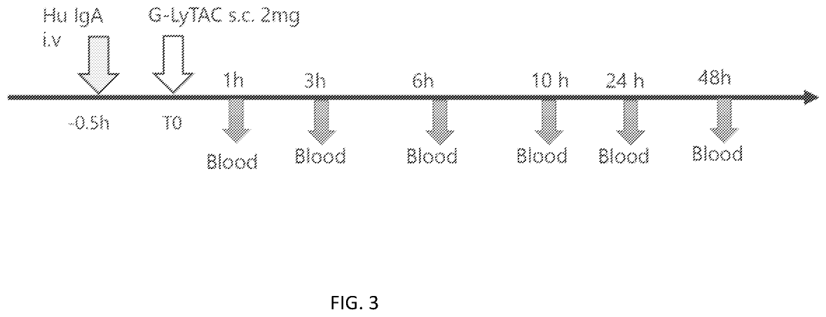

A typical O-glycosylation profile of the IgA1 hinge region is described in detail in Knoppova et al., particularly in FIG. 3 therein. Knoppova et al., 2017 is hereby incorporated by reference in its entirety.

As discussed in Knoppova 2017, normal serum IgA1 O-glycans consist predominantly of galactose-β1-3GalNAc dissaccharide, also known as T antigen, and its mono- or di-sialylated forms [NeuAcα2-3-galactose-β1-3GalNAc and NeuAcα2-3-galactose-β1-3(NeuAcat2-6)GalNAc, commonly described as sialyl-T (ST) antigen](see FIG. 3 of Knoppova 2017, left panel). After the initial addition of GalNAc to Ser/Thr residues, galactose is added by UDP-galactose:GalNAc-α-Ser/Thr β1,3-galactosyltransferase (C1GalT1). Galactose-β1,3GalNAc structures are subsequently modified by attaching sialic acid from CMP-N-acetylneuraminic acid (CMP-NeuAc) to galactose residues by the activity of galactose-β1,3GalNAc α2,3-sialyltransferase (ST3Gal) and/or to the GalNAc residues by activity of an α2,6-sialyltransferase (ST6GalNAc).

In some embodiments, a gd-IgA1 comprises an aberrant glycosylation profile as compared to a reference glycan profile. In some embodiments, a reference glycan profile disclosed herein comprises an O-glycosylation profile disclosed in FIG. 3 of Knoppova et al. In some embodiments, a reference glycan profile disclosed herein comprises a glycan profile described herein. In some embodiments, a gd-IgA1 comprises a glycan profile having a terminal GalNac (also referred to as the Tn antigen). In some embodiments, a gd-IgA1 comprises a glycan profile having a GalNac with an alpha2,6 linked sialic acid (also referred to as the STn antigen). In some embodiments, sialylation of a terminal GalNac blocks effective galactosylation resulting in a gd-IgA1 having an aberrant glycosylation profile.

An IgA1 hinge region is provided as SEQ ID NO: 1:

PVPSTPPTPSPSTPPTPSPSC.

In some embodiments, a IgA1 hinge region comprises the core sequence of:

(SEQ ID NO: 8)
VPSTPPTPSPSTPPTPSPS.

In some embodiments, the hinge region comprises nine O-glycosylation sites. In some embodiments, at least 1, 2, 3, 4, 5, or 6 of the O-glycosylation sites are occupied.

In some embodiments, a first moiety comprise one or more peptides specifically bind to a glycosylation-deficient IgA1 (gd-IgA1), or a fragment or a complex thereof. In some embodiments, the one or more peptides bind to a hinge region of gd-IgA1. In some embodiments, the one or more peptides bind to SEQ ID NO: 1 or a portion or a variant thereof. In some embodiments, the one or more peptides that bind to SEQ ID NO: 1 bind to one or more additional amino acid sequences besides SEQ ID NO: 1.

In some embodiments, the one or more peptides bind to and/or recognize a glycan profile on gd-IgA1. In some embodiments, the one or more peptides that specifically bind to gd-IgA1 recognize a glycan profile on gd-IgA1. In some embodiments, gd-IgA1 comprises a glycan profile that is different from a reference glycan profile of an IgA1. In some embodiments, gd-IgA1 comprises a glycan profile having at least one less glycan compared to a reference glycan profile of an IgA1.

In some embodiments, a reference glycan profile comprises an O-glycosylation profile. In some embodiments, a reference O-glycosylation profile comprises 1, 2, 3, 4, 5, or 6 O-glycan chains. In some embodiments, O-glycan chains of a reference O-glycosylation profile comprises N-acetylgalactosamine (GalNac). In some embodiments, an O-glycan chain further comprises a Galactose (Gal), a sialic acid or a combination thereof.

In some embodiments, a gd-IgA1 glycan profile comprises at least one less galactosylation compared to a reference glycan profile.

In some embodiments, a gd-IgA1 glycan profile comprises at least 5% less galactosylation compared to the reference glycan profile.

In some embodiments, a gd-IgA1 glycan profile comprises increased sialylation compared to the reference glycan profile.

In some embodiments, a gd-IgA1 glycan profile comprises a terminal GalNac (also referred to as the Tn antigen).

In some embodiments, a gd-IgA1 glycan profile comprises GalNac with an alpha-2,6 linked sialic acid (also referred to as the STn antigen).

In some embodiments, sialylation of a terminal GalNac blocks effective galactosylation.

In some embodiments, one or more peptides that specifically bind to gd-IgA1 recognize an epitope in a hinge region of gd-IgA1. In some embodiments, an altered glycan profile results in a conformational change in gd-IgA1. In some embodiments, a conformation change exposes a neoepitope that is recognized by the one or more peptides. In some embodiments, a neoepitope is not present in non-galactose deficient IgA1, e.g., IgA1 comprising a reference glycan profile. In some embodiments, a neoepitope is a linear epitope. In some embodiments, a neoepitope is a conformational epitope.

In some embodiments, one or more peptides that specifically bind to gd-IgA1 comprises an antibody agent. In some embodiments, an antibody agent comprises an antigen binding fragment. In some embodiments, an antibody agent comprises a full antibody, a Fab fragment, an scFv, a nanobody, a duobody, or a single domain antibody (e.g., a VHH). In some embodiments, an antibody agent comprises a VHH, e.g., a camelid VHH or a bivalent VHH.

Anti-Gd-IgA1 Autoantibodies and Peptides that Bind to Anti-Gd-IgA1

In IgA nephropathy autoimmunity, a gd-IgA1 autoantigens produces anti-gd-IgA1 autoantibodies. In some embodiments, anti-gd-IgA1 autoantibodies or immune complexes comprising the same form deposits in one or more tissues or organs. In some embodiments, anti-gd-IgA1 autoantibodies or immune complexes comprising the same contribute to and/or result in IgA nephropathy.

In some embodiments, an anti-gd-IgA1 autoantibody is an IgG antibody. In some embodiments, an anti-gd-IgA1 autoantibody is an IgA antibody. In some embodiments, an anti-gd-IgA1 autoantibody is an IgM antibody. In some embodiments, an anti-gd-IgA1 autoantibody is an IgD antibody. In some embodiments, an anti-gd-IgA1 autoantibody is an IgE antibody.

In some embodiments, a gd-IgA1 autoantigen is a gd-IgA1 polypeptide or a variant or fragment thereof. In some embodiments, a gd-IgA1 autoantigen is a glycan profile found on gd-IgA1. In some embodiments, an anti-gd-IgA1 autoantibody, a fragment, or a complex thereof is characterized in that it binds to a gd-IgA1 polypeptide or a variant or fragment thereof. In some embodiments, an anti-gd-IgA1 autoantibody, a fragment, or a complex thereof is characterized in that it binds to one or more glycans on gd-IgA1.

In some embodiments, an anti-gd-IgA1 antibody specifically binds to a glycan profile on gd-IgA1. In some embodiments, a glycan profile bound by an anti-gd-IgA1 is not present on a reference IgA1, e.g., an IgA1 from a healthy individual or an individual who is not at risk of developing IgAN. In some embodiments, a glycan profile bound by an anti-gd-IgA1 is a gd-IgA1 glycan profile described herein.

In some embodiments, the first moiety of a glycoengineered polypeptide disclosed herein comprises one or more peptides that specifically bind to one or more idiotopes of an anti-gd-IgA1 autoantibody, or a fragment thereof. In some embodiments, the one or more peptides comprise an anti-idiotypic antibody or a fragment (e.g., an antigen binding fragment) thereof.

In some embodiments, an anti-gd-IgA1 IgG comprises a mutation in a complementarity determining region 3 (CDR3) of an Ig heavy chain (IgH) variable region. In some embodiments, the mutation comprises an Alanine to Serine mutation. In some embodiments, the Alanine to Serine mutation occurs in a YCAR amino acid sequence or a YCAK amino acid sequence of a CDR3 IgH. Exemplary mutations in anti-gd-IgA1 autoantibodies are disclosed in U.S. Pat. No. 9,655,963, the entire contents of which are hereby incorporated by reference.

In some embodiments, the first moiety of a glycoengineered polypeptide disclosed herein comprises one or more peptides that specifically bind to a CDR3 IgH region of an anti-gd-IgA1 autoantibody or to a fragment thereof. In some embodiments, a CDR3 IgH region of an anti-gd-IgA1 autoantibody comprises a mutation, e.g., as described herein.

In some embodiments, the first moiety of a glycoengineered polypeptide disclosed herein comprises one or more peptides that specifically bind to a mutation in a CDR3 IgH region of an anti-gd-IgA1 autoantibody. In some embodiments, the one or more peptides bind to a YCAR amino acid sequence in a CDR3 IgH in which the Alanine is mutated to a Serine. In some embodiments, the one or more peptides bind to a YCAK amino acid sequence in a CDR3 IgH in which the Alanine is mutated to a Serine.

In some embodiments, one or more peptides that specifically bind to an anti-gd-IgA1 autoantibody binds to an IgG protein, or a fragment or a variant thereof. In some embodiments, the IgG is an IgG1, an IgG2, an IgG3, or an IgG4. In some embodiments, the IgG protein has a mutation in a CDR3 region.

In some embodiments, a wildtype IgG1 constant region polypeptide is provided as SEQ ID NO: 3.

ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK

In some embodiments, a wildtype IgG4 constant region is provided as SEQ ID NO: 4.

ASTKGPSVFPLAPCSRSTSESTAALGCLVKDYFPEPVTVSWNSGA
LTSGVHTFPAVLQSSGLYSLSSVVTVPSSSLGTKTYTCNVDHKPS
NTKVDKRVESKYGPPCPSCPAPEFLGGPSVFLFPPKPKDTLMISR
TPEVTCVVVDVSQEDPEVQFNWYVDGVEVHNAKTKPREEQFNSTY
RVVSVLTVLHQDWLNGKEYKCKVSNKGLPSSIEKTISKAKGQPRE
PQVYTLPPSQEEMTKNQVSLTCLVKGFYPSDIAVEWESNGQPENN
YKTTPPVLDSDGSFFLYSRLTVDKSRWQEGNVFSCSVMHEALHNH
YTQKSLSLSLGK

In some embodiments, the first moiety of a glycoengineered polypeptide disclosed herein comprises one or more peptides comprising an epitope recognized by anti-gd-IgA1 autoantibody, or a fragment thereof. In some embodiments, the first moiety of a glycoengineered polypeptide disclosed herein comprises one or more peptides that compete for binding with gd-IgA1 to an anti-gd-IgA1 autoantibody, or a fragment or complex thereof. In some embodiments, the first moiety of a glycoengineered polypeptide disclosed herein comprises one or more peptides that block binding of gd-IgA1 to an anti-gd-IgA1 autoantibody, or a fragment or complex thereof. In some embodiments, blocking binding of gd-IgA1 to an anti-gd-IgA1 autoantibody, or a fragment or complex thereof, reduces and/or prevents formation and/or deposition of immune complexes comprising anti-gd-IgA1 autoantibody in a tissue or organ.

In some embodiments, the first moiety of a glycoengineered polypeptide disclosed herein comprises one or more peptides that do not compete for binding with gd-IgA1 to an anti-gd-IgA1 autoantibody, or a fragment or complex thereof.

In some embodiments, a first moiety of a glycoengineered polypeptide disclosed herein comprises one or more peptides that specifically bind to an anti-gd-IgA1 autoantibody. In some embodiments, one or more peptides that specifically bind to an anti-gd-IgA1 autoantibody comprises a gd-IgA1 polypeptide, or a fragment or a variant thereof.

In some embodiments, one or more peptides that specifically bind to an anti-gd-IgA1 autoantibody comprises a gd-IgA1 polypeptide fragment. In some embodiments, a fragment comprises the sequence of SEQ ID NO: 1, or a fragment or a variant thereof. In some embodiments, a fragment comprises one or more additional amino acid residues to the 5′ and/or 3′ end of the sequence.

In some embodiments, a fragment comprises a gd-IgA1 polypeptide fragment having a glycan profile that is different from a reference glycan profile, e.g., as described herein. In some embodiments, a gd-IgA1 polypeptide fragment has a glycan profile having a terminal GalNac. In some embodiments, a gd-IgA1 polypeptide fragment has a glycan profile having a terminal sialylated GalNac. In some embodiments, a fragment comprises a gd-IgA1 amino acid sequence that leads to capping of GlcNac or GalNac with sialic acid.

In some embodiments, a fragment comprises an epitope that is recognized by anti-gd-IgA1 autoantibody. In some embodiments, an epitope comprises one or more glycans. In some embodiments, an epitope comprises a glycan profile, e.g., a gd-IgA1 glycan profile as described herein.

In some embodiments, a first moiety comprises a plurality of epitopes, e.g., the same or different epitopes. In some embodiments, a first moiety comprises a plurality of the same epitopes, e.g., epitopes recognized by an anti-gd-IgA1 autoantibody. In some embodiments, a first moiety comprises a plurality of different epitopes, e.g., epitopes recognized by different anti-gd-IgA1 autoantibodies. In some embodiments, the plurality of epitopes is separated by a linker, IRES or cleavage peptide.

In some embodiments, one or more peptides that specifically bind to an anti-gd-IgA1 autoantibody comprises a fragment, e.g., an epitope, that is not present or not accessible in a IgA1 polypeptide from a healthy individual or an individual not at risk of developing IgAN.

In some embodiments, a first moiety comprises 1, 2, 3, 4, 5, or more peptides that specifically bind to an anti-gd-IgA1 autoantibody.

In some embodiments, one or more peptides of a first moiety that specifically bind to an anti-gd-IgA1 autoantibody are the same, e.g., the one or more peptides have the same sequence. In some embodiments, the one or more peptides having the same sequence are separated by one or more intervening sequences (e.g., spacers and/or linkers). In some embodiments, the one or more peptides having the same sequence are not separated by one or more intervening sequences (e.g., spacers and/or linkers).

In some embodiments, one or more peptides of a first moiety that specifically bind to an anti-gd-IgA1 autoantibody are different, e.g., the one or more peptides do not have the same sequence. In some embodiments, the one or more peptides having different sequences are separated by one or more intervening sequences (e.g., spacers and/or linkers). In some embodiments, the one or more peptides different sequences are not separated by one or more intervening sequences (e.g., spacers and/or linkers).

In some embodiments, a linker separating one or more peptides of a first moiety comprises a Gly-Ser linker, or an EAAAK linker. In some embodiments, a linker comprises a (Gly-Gly-Gly-Gly-Ser)n linker, wherein n is an integer between 0 to 20.

In some embodiments, one or more peptides that specifically bind to an anti-gd-IgA1 autoantibody are each conjugated to a second moiety.

In some embodiments, one or more peptides that specifically bind to an anti-gd-IgA1 autoantibody are not each conjugated to the second moiety.

In some embodiments, one or more peptides that specifically bind to an anti-gd-IgA1 autoantibody are conjugated to each other, e.g., are situated on one polypeptide.

In some embodiments, one or more peptides that specifically bind to an anti-gd-IgA1 autoantibody are separated by a protease cleavage site or an IRES. In some embodiments, each of the one or more peptides is expressed as a separate peptide, e.g., translation as a separate peptide from an IRES or after cleavage of a protease cleavage site.

In some embodiments, one or more peptides that specifically bind to an anti-gd-IgA1 autoantibody are not separated by a protease cleavage site or an IRES, e.g., is expressed as a fusion protein.

In some embodiments, one or more peptides that specifically bind to an anti-gd-IgA1 autoantibody comprises an antibody agent. In some embodiments, the antibody agent comprises an antigen binding fragment. In some embodiments, the antibody agent comprises a full antibody, a Fab fragment, an scFv, a nanobody, a duobody, or a single domain antibody (e.g., a VHH). In some embodiments, one or more peptides that specifically bind to an anti-gd-IgA1 autoantibody comprises a VHH, e.g., a camelid VHH or a bivalent VHH.

### Second Moiety

A glycoengineered polypeptide disclosed herein comprises a first moiety that specifically binds to a target antibody or a fragment or a complex thereof; and a second moiety comprising one or more glycans conjugated to the first moiety at one or more glycosylation sites.

Without being bound by any particular theory, glycan engagement with endocytic carbohydrate binding proteins and receptors enables different biological pathways. These essential biological pathways are involved in modulating immune responses, mediating protein clearance, protein turnover, and controlling trafficking of soluble glycoproteins, glycolipids and any natural molecule containing a glycan moiety. The glycan-receptor interaction is determined by the glycan structure. Glycan binding receptors are highly diverse and can be exploited by glycoengineering to develop novel therapeutics based on the concept of glycan-mediated protein degradation to treat different diseases, which include but are not limited to autoimmune disorders as disclosed herein.

Further without being bound by any particular theory a glycoengineered polypeptide comprising a second moiety having one or more glycans, as described herein, is expected to activate natural degradation pathways.

In some embodiments, a second moiety of a glycoengineered polypeptide disclosed herein comprises one or more glycans and specifically binds to one or more endocytic receptors. Endocytic receptors as described herein capture glycoproteins via specific glycan structures to mediate degradation, e.g., lysosomal degradation. Endocytic receptors are ubiquitous in human and can be found on different cells.

In some embodiments, an endocytic receptor is or comprises an endocytic lectin. In some embodiments, the endocytic receptor is chosen from: an asialoglycoprotein receptor (ASGPR); a mannose binding receptor, a Cluster of Differentiation 206 (CD206) receptor; a DC-SIGN (Cluster of Differentiation 209 or CD209) receptor; a C-Type Lectin Domain Family 4 Member G (LSECTin) receptor; a macrophage inducible Ca2+-dependent lectin receptor (Mincle); a L-SIGN CD209L receptor; dectin-1; dectin-2, langerin, macrophage mannose 2 receptor, BDCA-2, DCIR, MBL, MDL, MICL, CLEC2, CLEC10, DNGR1, CLEC12B, DEC-205, and mannose 6 phosphate receptor (M6PR), or a combination thereof.

In some embodiments, provided herein is a glycoengineered polypeptide comprising a first moiety that specifically binds to a target antibody and a second moiety comprising a glycan comprising terminal GlcNAc.

In some embodiments, provided herein is a glycoengineered polypeptide comprising a first moiety that specifically binds to a target antibody and a second moiety comprising a glycan comprising terminal GalNAc.

In some embodiments, provided herein is a glycoengineered polypeptide comprising a first moiety that specifically binds to a target antibody and a second moiety comprising a glycan comprising terminal Gal.

In some embodiments, a glycoengineered polypeptide provided herein can comprise (i) a binding specificity to one or more target antibodies and (ii) one or more N-glycan(s) with binding specificities to one or more endocytic receptor(s).

In some embodiments, a glycoengineered polypeptide comprises one type of N-glycan with binding specificity to one type of endocytic receptor.

In some embodiments, a glycoengineered polypeptide comprises one or more N-glycosylation sites in a first moiety. In some embodiments, one or more N-glycosylation sites in a first moiety are native N-glycosylation sites. In some embodiments, one or more N-glycosylation sites in a first moiety are engineered N-glycosylation sites. In some embodiments, a glycoengineered polypeptide comprises one or more native N-glycosylation sites and one or more engineered N-glycosylation sites.

In some embodiments, a second moiety comprising one or more glycans is conjugated, e.g., linked, to a first moiety at one or more N-glycosylation sites.

In some embodiments, a glycoengineered polypeptide comprises 1, 2, 3, 4, 5, 6, 7, 8, 9, 10 or more N-glycosylation sites (or glycosites; such as an N-glycosylation consensus sequence). These N-glycosylation sites can be glycosylated by an N-glycan such that the resulting glycoengineered bifunctional binding protein can engage with or bind to 1, 2, 3, 4, 5, 6, 7, 8, 9, 10 or more endocytic receptor molecules.

In some embodiments, a glycoengineered polypeptide comprises two types of N-glycans with binding specificities to two different endocytic receptors. In certain embodiments, a glycoengineered polypeptide provided herein can comprise 1, 2, 3, 4, 5, 6, 7, 8, 9, 10 or more polypeptide chains. Each chain can be produced in a different cell line. In certain embodiments, the glycoengineered polypeptide can be an antibody and one type of N-glycan is on the Fc domain and another type of N-glycan is on the Fab domain (eg, the variable regions) of the antibody.

In some embodiments, a glycoengineered polypeptide comprises: (i) a first type of N-glycan with binding specificity to a first endocytic receptor wherein the first type of N-glycan is present at 1, 2, 3, 4, 5, 6, 7, 8, 9, 10 or more glycosites thus engaging with or binding to 1, 2, 3, 4, 5, 6, 7, 8, 9, 10 or more molecular of the first endocytic carbohydrate-binding protein or receptor; and (ii) a second type of N-glycan with binding specificity to a second endocytic receptor wherein the second N-glycan is present at 1, 2, 3, 4, 5, 6, 7, 8, 9, 10 or more glycosites so that a single bifunctional binding protein can engage with or bind to 1, 2, 3, 4, 5, 6, 7, 8, 9, 10 or more molecules of the second endocytic receptor(s).

In some embodiments, a glycoengineered polypeptide comprises: (i) a first type of N-glycan with binding specificity to a first endocytic receptor wherein the first type of N-glycan is present at 1, 2, 3, 4, 5, 6, 7, 8, 9, 10 or more glycosites thus engaging with or binding to 1, 2, 3, 4, 5, 6, 7, 8, 9, 10 or more molecular of the first endocytic receptor; (ii) a second type of N-glycan with binding specificity to a second endocytic receptor wherein the second N-glycan is present at 1, 2, 3, 4, 5, 6, 7, 8, 9, 10 or more glycosites so that a single bifunctional binding protein can engage with or bind to 1, 2, 3, 4, 5, 6, 7, 8, 9, 10 or more molecules of the second endocytic receptor(s); and (iii) a third type of N-glycan with binding specificity to a third endocytic receptor wherein the third N-glycan is present at 1, 2, 3, 4, 5, 6, 7, 8, 9, 10 or more glycosites so that a single bifunctional binding protein can engage with or bind to 1, 2, 3, 4, 5, 6, 7, 8, 9, 10 or more molecules of the third endocytic receptor(s).

In some embodiments, a glycoengineered polypeptide provided herein has 1, 2, 3, 4, 5, 6, 7, 8, 9, 10 or more glycosites. In some embodiments, in a population of glycoengineered polypeptides, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% of the glycosites in the population at one specific position are glycosylated. In certain embodiments, in a population of glycoengineered polypeptides, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% of the glycosites in the population are glycosylated. N-glycans that can be present at the glycosites of the glycoengineered polypeptide provided herein are described herein.

In some embodiments, a glycosite is an N-glycosylation consensus sequence. The consensus sequence can be N-X-S/T, or N-X-C, wherein X is any amino acid except proline.

In some embodiments, a glycosite is or comprises the sequence of

(SEQ ID NO: 37)
GGGGANSTAPAPAPA.

In some embodiments, an N-glycan is conjugated to the glycoengineered polypeptide at at least one, two, three, or four N-glycosylation sites.

In some embodiments, an N-glycan is conjugated to the glycoengineered polypeptide at one, two, three, or four N-glycosylation sites.

In some embodiments, an N-glycosylation site is naturally occurring.

In some embodiments, an N-glycosylation site is engineered into the amino acid sequence of the first moiety.

In certain embodiments, one or more of the N-glycosylation sites are engineered into the amino acid sequence of the first moiety of the glycoengineered polypeptide (i.e. one or more of the N-glycosylation sites are not present in a wild-type, or naturally occurring form of the first moiety). In certain embodiments, at least one of the N-glycosylation sites is engineered into the amino acid sequence of the first moiety of the glycoengineered polypeptide. In certain embodiments, at least two of the N-glycosylation sites are engineered into the amino acid sequence of the first moiety of the glycoengineered polypeptide. In certain embodiments, at least three of the N-glycosylation sites are engineered into amino acid sequence of the first moiety of the glycoengineered polypeptide. In certain embodiments, at least four of the N-glycosylation sites are engineered into the amino acid sequence of the first moiety of the glycoengineered polypeptide. In certain embodiments, one or more of the engineered N-glycosylation sites are glycotags fused to the N- and/or C-terminus of the amino acid sequence of the first moiety of the glycoengineered polypeptide via a peptide linker. In certain embodiments, a glycotag is fused to the N-terminus of first moiety of the glycoengineered polypeptide. In certain embodiments, a glycotag is fused to the C-terminus of first moiety of the glycoengineered polypeptide. In certain embodiments, a glycotag is fused to the N- and the C-terminus of first moiety of the glycoengineered polypeptide. In certain embodiments, one or more of the N-glycosylation sites are natural N-glycosylation sites (i.e. one or more of the N-glycosylation sites are present in a wild-type, or naturally occurring form of the first moiety). In certain embodiments, at least one of the N-glycosylation sites is a natural N-glycosylation site. In certain embodiments, at least two of the N-glycosylation sites are natural N-glycosylation sites.

In some embodiments, provided herein is a glycoengineered polypeptide that specifically binds to a target antibody associated with a disease disclosed herein, comprising a first moiety and a second moiety. In some embodiments, provided herein is a glycoengineered polypeptide comprising a first moiety that specifically binds to a target antibody associated with a disease disclosed herein and a second moiety that binds specifically to an endocytic receptor, wherein the second moiety comprises a glycan structure.

In some embodiments, provided herein is a glycoengineered polypeptide comprising a first moiety that specifically binds to a target protein and a second moiety comprising an N-glycan selected from the group consisting of GlcNAc2Man3GlcNAc2, GalNAc2GlcNAc2Man3 GlcNAc2, Gal2GlcNAc2Man3GlcNAc2, Man3 GlcNAc, GlcNAc 1Man3 GlcNAc2, Gal2GlcNAc2Man3 GlcNAc2, Gal 1 GlcNAc2Man3 GlcNAc2, GalNAc 1 GlcNAc2Man3 GlcNAc2, GlcNAc3Man3 GlcNAc2, GlcNAc4Man3 GlcNAc2, Gal3GlcNAc3Man3 GlcNAc2, GalNAc3 GlcNAc3Man3 GlcNAc2, GalNAc4GlcNAc4Man3GlcNAc2, Gal4GlcNAc4Man3GlcNAc2, or Man-6-P-N-glycan.

In some embodiments, increasing the number of glycan structures on a glycoengineered polypeptide increases the rate of lysosomal degradation as compared to an otherwise similar glycoengineered polypeptide with fewer glycan structures.

In some embodiments, the number of glycan structures on a glycoengineered polypeptide disclosed herein is 1 or more, 2 or more, 3 or more, 4 or more, 5 or more, 6 or more, 7 or more, 8 or more, 9 or more or 10 or more glycan structures.

In some embodiments, a glycoengineered polypeptide disclosed herein comprises a glycan structure having a monoantennary structure.

In some embodiments, a glycoengineered polypeptide disclosed herein comprises a glycan structure having a biantennary structure.

In some embodiments, a glycoengineered polypeptide disclosed herein comprises a glycan structure having a triantennary structure.

In some embodiments, a glycoengineered polypeptide disclosed herein comprises a glycan structure having a tetraantennary structure.

In some embodiments, the glycan structure comprises a biantennary structure. In some embodiments, the glycan structure comprises a biantennary GalNAc. In some embodiments, the biantennary GalNac binds to an asialoglycoprotein receptor (ASGPR) or a fragment or variant thereof, or a complex comprising ASGPR.

In some embodiments, the N-glycan has a structure of:

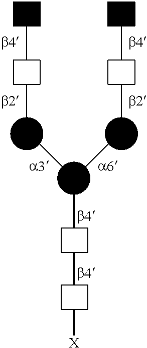

wherein the black square represents an N-acetyl galactosamine (GalNAc), the white square represents an N-acetylglucosamine (GlcNAc) residue and the black circle represents a mannose (Man) residue, and wherein X represents an amino acid residue of the first moiety.

In some embodiments, the N-glycan specifically binds to one or more endocytic receptors, e.g., that mediate lysosomal degradation. In some embodiments, the N-glycan specifically binds to ASGPR.

In some embodiments, the endocytic receptor is or comprises ASGPR or a fragment or variant thereof, or a complex comprising ASGPR. In some embodiments, when the endocytic receptor is ASGPR, the glycan structure of the second moiety comprises a terminal GalNac.

ASGPR-mediated degradation in the hepatocyte has many applications. ASPGR binding to the N-glycan structure disclosed herein can result in the selective degradation of one or more target antibodies (e.g., as disclosed herein). By way of example, ASGPR-mediated degradation can lead to removal of cytokines, chemokines and hormones. Additionally, ASGPR-mediated degradation can be used for the delivery of the target molecules to the hepatocyte endosome. Thus, ASGPR-mediated degradation is applicable for various diseases, while limiting systemic toxicity.

In certain embodiments, the 1, 2, 3, 4, 5, 6, 7, 8, 9, 10 or more N-glycosylation sites can be glycosylated by the N-glycan such that the resulting glycoengineered polypeptide can engage with or bind to 1, 2, 3, 4, 5, 6, 7, 8, 9, 10 or more endocytic receptor molecules. In certain embodiments, the glycoengineered polypeptide is glycosylated by the N-glycan at at least 2, at least 3, at least 4, at least 5, at least 6, at least 7, at least 8, at least 9, or at least 10 of the N-glycosylation sites. In certain embodiments, the glycoengineered polypeptide is glycosylated by the N-glycan at at least 2 N-glycosylation sites. In certain embodiments, the glycoengineered polypeptide is glycosylated by the N-glycan at at least 3 N-glycosylation sites. In certain embodiments, the glycoengineered polypeptide is glycosylated by the N-glycan at at least 4 N-glycosylation sites. In certain embodiments, the glycoengineered polypeptide is glycosylated by the N-glycan at at least 5 N-glycosylation sites. In certain embodiments, the glycoengineered polypeptide is glycosylated by the N-glycan at at least 6 N-glycosylation sites. In certain embodiments, the glycoengineered polypeptide is glycosylated by the N-glycan at at least 7 N-glycosylation sites. In certain embodiments, the glycoengineered polypeptide is glycosylated by the N-glycan at at least 8 N-glycosylation sites. In certain embodiments, the glycoengineered polypeptide is glycosylated by the N-glycan at at least 9 N-glycosylation sites. In certain embodiments, the glycoengineered polypeptide is glycosylated by the N-glycan at at least 10 N-glycosylation sites.

In certain embodiments, the glycoengineered polypeptide is glycosylated by the N-glycan at 2 N-glycosylation sites. In certain embodiments, the glycoengineered polypeptide is glycosylated by the N-glycan at 3 N-glycosylation sites. In certain embodiments, the glycoengineered polypeptide is glycosylated by the N-glycan at 4 N-glycosylation sites. In certain embodiments, the glycoengineered polypeptide is glycosylated by the N-glycan at 5 N-glycosylation sites. In certain embodiments, the glycoengineered polypeptide is glycosylated by the N-glycan at 6 N-glycosylation sites. In certain embodiments, the glycoengineered polypeptide is glycosylated by the N-glycan at 7 N-glycosylation sites. In certain embodiments, the glycoengineered polypeptide is glycosylated by the N-glycan at 8 N-glycosylation sites. In certain embodiments, the glycoengineered polypeptide is glycosylated by the N-glycan at 9 N-glycosylation sites. In certain embodiments, the glycoengineered polypeptide is glycosylated by the N-glycan at 10 N-glycosylation sites. In certain embodiments, the glycoengineered polypeptide is glycosylated at an Asn amino acid residue of the glycoengineered polypeptide. In certain embodiments, the N-glycosylation site is an N-glycosylation consensus sequence. In certain embodiments, the N-glycosylation site comprises a consensus sequence of N-X-S/T or N-X-C, wherein X is any amino acid except proline.

In certain embodiments, at least 10%, at least 20%, at least 30%, at least 40%, at least 50%, at least 60%, at least 70%, at least 80%, at least 90%, at least 95, or at least 98% of the N-glycosylation sites are occupied by an N-glycan. In certain embodiments, at least 10%, at least 20%, at least 30%, at least 40%, at least 50%, at least 60%, at least 70%, at least 80%, at least 90%, at least 95, or at least 98% of the N-glycosylation sites are occupied by an N-glycan of the structure:

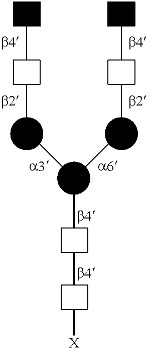

linked to the glycoengineered polypeptide at one or more N-glycosylation sites, wherein the black square represents an N-acetyl galactosamine (GalNAc), the white square represents an N-acetylglucosamine (GlcNAc) residue the black circle represents a mannose (Man) residue, and X represents an amino acid residue of the glycoengineered polypeptide. In certain embodiments, at least 10% of the N-glycosylation sites are occupied by the N-glycan. In certain embodiments, at least 20% of the N-glycosylation sites are occupied by the N-glycan. In certain embodiments, at least 30% of the N-glycosylation sites are occupied by the N-glycan. In certain embodiments, at least 40% of the N-glycosylation sites are occupied by the N-glycan. In certain embodiments, at least 50% of the N-glycosylation sites are occupied by the N-glycan. In certain embodiments, at least 60% of the N-glycosylation sites are occupied by the N-glycan. In certain embodiments, at least 70% of the N-glycosylation sites are occupied by the N-glycan. In certain embodiments, at least 80% of the N-glycosylation sites are occupied by the N-glycan. In certain embodiments, at least 90% of the N-glycosylation sites are occupied by the N-glycan. In certain embodiments, at least 95% of the N-glycosylation sites are occupied by the N-glycan. In certain embodiments, at least 98% of the N-glycosylation sites are occupied by the N-glycan.

In certain embodiments, the N-glycan is linked to the glycoengineered polypeptide at least one N-glycosylation site. In certain embodiments, the N-glycan is linked to the glycoengineered polypeptide at least two N-glycosylation sites. In certain embodiments, the N-glycan is linked to the glycoengineered polypeptide at one, two, three, or four N-glycosylation sites. In certain embodiments, the N-glycan is linked to the glycoengineered polypeptide at one N-glycosylation site. In certain embodiments, the N-glycan is linked to the glycoengineered polypeptide at two N-glycosylation sites. In certain embodiments, the N-glycan is linked to the glycoengineered polypeptide at three N-glycosylation sites. In certain embodiments, the N-glycan is linked to the glycoengineered polypeptide at four N-glycosylation sites. In certain embodiments, the N-glycan is linked to the glycoengineered polypeptide at an Asn amino acid residue of the glycoengineered polypeptide. In certain embodiments, the N-glycan is linked to the glycoengineered polypeptide at an N-glycosylation consensus sequence. In certain embodiments, the N-glycan is linked to the glycoengineered polypeptide at a consensus sequence of N-X-S/T or N-X-C, wherein X is any amino acid except proline.

In certain embodiments, the glycoengineered polypeptide glycoengineered polypeptide glycoengineered polypeptide comprises two different N-glycans (i.e. a first and a second N-glycan), wherein each N-glycan is independently linked to the glycoengineered polypeptide at 1, 2, 3, 4, 5, 6, 7, 8, 9, 10 or more N-glycosylation sites, and wherein one of the N-glycans (i.e. the first N-glycan) has the structure:

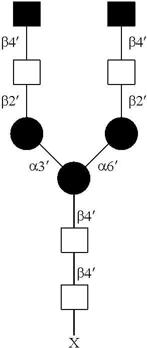

wherein the black square represents an N-acetyl galactosamine (GalNAc), the white square represents an N-acetylglucosamine (GlcNAc) residue the black circle represents a mannose (Man) residue, and X represents an amino acid residue of the glycoengineered polypeptide. In certain embodiments, the different N-glycans specifically bind to different endocytic receptors. In certain embodiments, the first N-glycan specifically binds to ASGPR. In certain embodiments, the other N-glycan is an N-glycan described in PCT/EP2022/057556, which is incorporated herein by reference in its entirety. In certain embodiments, the first N-glycan is larger than the second N-glycan. In other embodiments, the first N-glycan is smaller than the second N-glycan. In certain embodiments, the N-glycosylation sites predominantly or exclusively occupied by the larger N-glycan are more sterically accessible than the N-glycosylation sites predominantly or exclusively occupied by the smaller N-glycan. In certain embodiments, the other N-glycan is A2. In certain embodiments, the other N-glycan is A1GalNAc1 or A2GalNAc1. In certain embodiments, the N-glycans are linked to the glycoengineered polypeptide at an Asn amino acid residue of the glycoengineered polypeptide. In certain embodiments, the N-glycans are linked to the glycoengineered polypeptide at an N-glycosylation consensus sequence. In certain embodiments, the N-glycans are linked to the glycoengineered polypeptide at a consensus sequence of N-X-S/T or N-X-C, wherein X is any amino acid except proline. In certain embodiments, the first N-glycan is linked to the glycoengineered polypeptide at 1, 2, 3, 4, 5, 6, 7, 8, 9, 10 or more N-glycosylation sites, and the second N-glycan is linked to the glycoengineered polypeptide at 1, 2, 3, 4, 5, 6, 7, 8, 9, 10 or more N-glycosylation sites.

In certain embodiments, the glycoengineered polypeptide further comprises a third N-glycan, wherein the third N-glycan is linked to the glycoengineered polypeptide at 1, 2, 3, 4, 5, 6, 7, 8, 9, 10 or more N-glycosylation sites. In certain embodiments, the third N-glycan specifically binds to a different endocytic receptor than the first and/or second N-glycan. In certain embodiments, the third N-glycan is an N-glycan described in PCT/EP2022/057556, which is incorporated herein by reference in its entirety. In certain embodiments, the third N-glycan is A2. In certain embodiments, the third N-glycan is A1GalNAc1 or A2GalNAc1. In certain embodiments, the third N-glycan is linked to the glycoengineered polypeptide at an Asn amino acid residue of the glycoengineered polypeptide. In certain embodiments, the third N-glycan is linked to the glycoengineered polypeptide at an N-glycosylation consensus sequence. In certain embodiments, the third N-glycan is linked to the glycoengineered polypeptide at a consensus sequence of N-X-S/T or N-X-C, wherein X is any amino acid except proline.

In certain embodiments, the second and/or third N-glycan specifically bind to an endocytic lectin. In some embodiments, the endocytic lectin is a mannose binding receptor. In some embodiments, the endocytic lectin is a Cluster of Differentiation 206 (CD206) receptor. In some embodiments, the endocytic lectin is a DC-SIGN (Cluster of Differentiation 209 or CD209) receptor. In some embodiments, the endocytic lectin is a C-Type Lectin Domain Family 4 Member G (LSECTin) receptor. In some embodiments, the endocytic lectin is a macrophage inducible Ca²⁺-dependent lectin receptor (Mincle). In some embodiments, the endocytic receptor is L-SIGN CD209L. In some embodiments, the endocytic receptor is asialoglycoprotein (ASGPR). In some embodiments, the endocytic receptor is dectin-1. In some embodiments, the endocytic receptor is dectin-2. In some embodiments, the endocytic receptor is langerin. In some embodiments, the second and/or third N-glycan specifically bind to a receptor selected from the group consisting of macrophage mannose 2 receptor, BDCA-2, DCIR, MBL, MDL, MICL, CLEC2, CLEC10, DNGR1, CLEC12B, DEC-205, and mannose 6 phosphate receptor (M6PR).

CD206 is a C-type lectin and phagocytic/endocytic recycling and signaling receptor. CD206 is expressed primarily by M2 anti-inflammatory macrophages, dendritic cells, and live sinusoidal endothelial cells. DC-SIGN is a non-recycling, signaling receptor that targets both the ligand and receptor to the lysosome for degradation. LSECTin is expressed on liver sinusoidal endothelial cells.

In certain embodiments, the glycoengineered polypeptide glycoengineered polypeptide glycoengineered polypeptide is glycosylated at two or more N-glycosylation sites by an N-glycan of the structure:

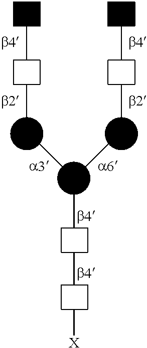

wherein the black square represents an N-acetyl galactosamine (GalNAc), the white square represents an N-acetylglucosamine (GlcNAc) residue the black circle represents a mannose (Man) residue, and X represents an amino acid residue of the glycoengineered polypeptide, and wherein two of the N-glycosylation sites are separated by at least 5, at least 10, at least 20, at least 30, at least 40, at least 50, at least 60, at least 70, at least 80, at least 90, or at least 100 amino acids. In certain embodiments, the N-glycan is linked to the glycoengineered polypeptide at two N-glycosylation sites separated by a distance of about 5-10, about 10-20, about 20-30, about 30-40, about 40-50, about 50-60, about 60-70, about 70-80, about 80-90, about 90-100, about 100-150, about 150-200, or about 200-300 amino acids. In certain embodiments, the amino acid separation between the N-glycosylation sites is the number of amino acids between the terminal amino acids of the N-glycosylation consensus sequence. Without being bound by theory, the glycoengineered polypeptide folds in space and, thus, has a three-dimensional geometry in addition to its primary amino acid structure. Also without being bound by theory, this three-dimensional geometry, including the position of the N-glycan is not static but dynamic (see, for example, Re, S., et al Biophysical Reviews, 4, 179-187 (2012)). Notwithstanding, in certain embodiments, the distance between N-glycosylation sites and/or N-glycans on a glycoengineered polypeptide may be from an equilibrium geometry of the glycoengineered polypeptide, as determined by any standard means known in the art, including for example computational modelling studies. In certain embodiments, the N-glycan is linked to the glycoengineered polypeptide at two N-glycosylation sites separated by a distance of at least 1.0 nm. In certain embodiments, the N-glycan is linked to the glycoengineered polypeptide at two N-glycosylation sites separated by a distance of about 1.0-5.0 nm. In certain embodiments, the N-glycan is linked to the glycoengineered polypeptide at two N-glycosylation sites separated by a distance of about 1.5-3.0 nm. In certain embodiments, the N-glycan is linked to the glycoengineered polypeptide at two N-glycosylation sites separated by a distance of about 1.5-2.5 nm. In certain embodiments, the N-glycan is linked to the glycoengineered polypeptide at three N-glycosylation sites each separated by a distance of about 1.0-5.0 nm. In certain embodiments, the N-glycan is linked to the glycoengineered polypeptide at three N-glycosylation sites each separated by a distance of about 1.5-3.0 nm. In certain embodiments, the N-glycan is linked to the glycoengineered polypeptide at three N-glycosylation sites each separated by a distance of about 1.5-2.5 nm. In certain embodiments, the N-glycans are separated by a distance of at least 1.0 nm. In certain embodiments, the N-glycans are separated by a distance of about 1.0 to about 5.0 nm. In certain embodiments, the N-glycans are separated by a distance of about 1.5 to about 2.5 nm. In certain embodiments, the distance between the N-glycosylation sites and/or N-glycans is chosen to minimize steric hindrance, for example between the glycoengineered polypeptide (s), the target protein(s), and/or the ASGPR receptor(s). In certain embodiments, the distance between the N-glycosylation sites and/or N-glycans is chosen based on the separation of ASGPR receptors on a cell surface. In certain embodiments, the distance between the N-glycosylation sites and/or N-glycans is chosen to be similar (e.g. no more than twice, or no less than half) to the separation of ASGPR receptors on a cell surface. In certain embodiments, the N-glycan is linked to the glycoengineered polypeptide at an Asn amino acid residue of the glycoengineered polypeptide. In certain embodiments, the N-glycan is linked to the glycoengineered polypeptide at an N-glycosylation consensus sequence. In certain embodiments, the N-glycan is linked to the glycoengineered polypeptide at a consensus sequence of N-X-S/T or N-X-C, wherein X is any amino acid except proline.

### Nucleic Acid Sequences Encoding Glycoengineered Polypeptides

The present disclosure, among other things, provides nucleic acids encoding glycoengineered polypeptides as described herein.

In some embodiments, a nucleic acid is or comprises single stranded DNA (e.g., as in certain viral vectors). In some embodiments, a nucleic acid is or comprises double stranded DNA (e.g., as in certain viral vectors and/or certain plasmids). In some embodiments, a nucleic acid is or comprises RNA (e.g., as in certain viral vectors and/or as in mRNA therapeutics), etc.

Nucleic acids encoding glycoengineered polypeptides may be modified to include codons that are optimized for expression in a particular cell type (e.g., a Leishmania cell) or organism. Codon optimized sequences are synthetic sequences, and preferably encode an identical polypeptide (or biologically active fragment of a full length polypeptide which has substantially the same activity as the full length polypeptide) encoded by a non-codon optimized parent polynucleotide. In some embodiments, a coding region of a nucleic acids encoding glycoengineered polypeptides described herein, in whole or in part, may include an altered sequence to optimize codon usage for a particular cell type (e.g., a eukaryotic or prokaryotic cell). For example, a coding sequence for an antibody agent (e.g., antigen binding fragment) as described herein may be optimized for expression in a bacterial cells. Alternatively, the coding sequence may be optimized for expression in a mammalian cell (e.g., a CHO cell). Such a sequence may be described as a codon-optimized sequence.

Nucleic acid constructs of the present disclosure may be inserted into an expression vector or viral vector by methods known to the art, and nucleic acids may be operably linked to an expression control sequence. A vector comprising any nucleic acids or fragments thereof described herein is further provided by the present disclosure. Any nucleic acids or fragments thereof described herein can be cloned into any suitable vector and can be used to transform or transfect any suitable host (e.g., Leishmania host cell). Selection of vectors and methods to construct them are commonly known to persons of ordinary skill in the art.

In some embodiments, nucleic acids and vectors of the present disclosure are isolated and/or purified. The present disclosure also provides a composition comprising an isolated or purified nucleic acid, optionally in the form of a vector. Isolated nucleic acids and vectors may be prepared using standard techniques known in the art including, for example, alkali/SDS treatment, CsCl binding, column chromatography, agarose gel electrophoresis, and/or other techniques well known in the art. The composition can comprise other components as described further herein.

Any method known to one skilled in the art for the insertion of nucleic acids into a vector may be used to construct expression vectors encoding a glycoengineered polypeptide described herein under control of transcriptional and/or translational control signals. These methods may include in vitro recombinant DNA and synthetic techniques and in vivo recombination (see, e.g., Sambrook et al., Molecular Cloning, a Laboratory Manual, 2d edition, Cold Spring Harbor Press, Cold Spring Harbor, N.Y. (1989); and Ausubel et al., Current Protocols in Molecular Biology, Greene Publishing Associates and John Wiley & Sons, New York, N.Y. (1994), each of which is hereby incorporated by reference in its entirety).

### Compositions and Pharmaceutical Compositions

A composition disclosed herein may comprise and/or deliver one or more glycoengineered polypeptides disclosed herein or nucleic acids encoding one or more glycoengineered polypeptides disclosed herein.

In some embodiments, a composition disclosed herein comprises a glycoengineered polypeptide comprising a first moiety and a second moiety. In some embodiments, a composition disclosed herein comprises a plurality of glycoengineered polypeptides comprising a first moiety and a second moiety.

In some embodiments, a composition comprising a plurality of glycoengineered polypeptides comprises 1, 2, 3, 4, 5, or more glycoengineered polypeptides comprising a first moiety that binds to a target antibody (e.g., a first moiety that binds to the same target antibody).

In some embodiments, a composition comprising a plurality of glycoengineered polypeptides comprises glycoengineered polypeptides having a first moiety that binds to a gd-IgA1 or a fragment thereof. In some embodiments, the glycoengineered polypeptides in the plurality each comprise the same first moiety. In some embodiments, the glycoengineered polypeptides in the plurality each comprise a different first moiety (e.g., a first glycoengineered polypeptide comprises a first moiety that binds to a IgA1 or a fragment thereof, a second glycoengineered polypeptide comprises a different first moiety that binds to a IgA1 or a fragment thereof, etc.).

In some embodiments, a composition comprising a plurality of glycoengineered polypeptides comprises glycoengineered polypeptides having a first moiety that binds to a gd-IgA1 or a fragment thereof. In some embodiments, the glycoengineered polypeptides in the plurality each comprise the same first moiety. In some embodiments, the glycoengineered polypeptides in the plurality each comprise a different first moiety (e.g., a first glycoengineered polypeptide comprises a first moiety that binds to a gd-IgA1 or a fragment thereof, a second glycoengineered polypeptide comprises a different first moiety that binds to a gd-IgA1 or a fragment thereof, etc.).

In some embodiments, a composition comprising a plurality of glycoengineered polypeptides comprises glycoengineered polypeptides having a first moiety that binds to an anti-gd-IgA1 autoantibody or a fragment thereof. In some embodiments, the glycoengineered polypeptides in the plurality each comprise the same first moiety. In some embodiments, the glycoengineered polypeptides in the plurality each comprise a different first moiety (e.g., a first glycoengineered polypeptide comprises a first moiety that binds to an anti-gd-IgA1 autoantibody or a fragment thereof, a second glycoengineered polypeptide comprises a different first moiety that binds to an anti-gd-IgA1 autoantibody or a fragment thereof, etc.).

In some embodiments, a composition comprising a plurality of glycoengineered polypeptides comprises 1, 2, 3, 4, 5, or more glycoengineered polypeptides each comprising a first moiety that binds to a different target antibody (e.g., an IgA1 or a fragment thereof, or an gd-IgA1 or a fragment thereof or an anti-gd-IgA1 autoantibody or a fragment thereof). In some embodiments, a composition comprising a plurality of glycoengineered polypeptides comprises a first glycoengineered polypeptide comprising a first moiety that binds to a target antibody (e.g., an IgA1 or a fragment thereof), a second glycoengineered polypeptide comprising a first moiety that binds to a different target antibody (e.g., a gd-IgA1 or a fragment thereof) and a third glycoengineered polypeptide comprising a first moiety that binds to a different target antibody (e.g., an anti-gd-IgA1 autoantibody or a fragment thereof).

In some embodiments, a glycoengineered polypeptide disclosed herein (i) a first glycoengineered polypeptide comprising a first moiety that specifically binds to an IgA1 or a fragment or a complex thereof, (ii) a second glycoengineered polypeptide comprising a first moiety that specifically binds to a gd-IgA1 or a fragment or a complex thereof; and (iii) a third glycoengineered polypeptide comprising a first moiety that specifically binds to an anti-gd-IgA1 autoantibody or a fragment or a complex thereof.

In some embodiments, a glycoengineered polypeptide disclosed herein (i) a first glycoengineered polypeptide comprising a first moiety that specifically binds to an IgA1 or a fragment or a complex thereof, and (ii) a second glycoengineered polypeptide comprising a first moiety that specifically binds to a target antibody other than an IgA1, or a fragment or a complex thereof.

In some embodiments, a glycoengineered polypeptide disclosed herein (i) a first glycoengineered polypeptide comprising a first moiety that specifically binds to a gd-IgA1 or a fragment or a complex thereof, and (ii) a second glycoengineered polypeptide comprising a first moiety that specifically binds to a target antibody other than a gd-IgA1, or a fragment or a complex thereof.

In some embodiments, a glycoengineered polypeptide disclosed herein (i) a first glycoengineered polypeptide comprising a first moiety that specifically binds to an anti-gd-IgA1 autoantibody or a fragment or a complex thereof, and (ii) a second glycoengineered polypeptide comprising a first moiety that specifically binds to a target antibody other than an anti-gd-IgA1 autoantibody, or a fragment or a complex thereof.

In some embodiments, ratio of the first glycoengineered polypeptide to the second glycoengineered polypeptide is about 1:9, about 1:8, about 1:7, about 1:6, about 1:5, about 1:4, about 1:3, about 1:2.5, about 1:2, about 1:1.5, about 1:1, about 9:1, about 8:1, about 7:1, about 6:1, about 5:1, about 4:1, about 3:1, about 2.5:1 about, 2:1, or about 1.5:1.

In some embodiments, the ratio of the first glycoengineered polypeptide to the second glycoengineered polypeptide is about 1:5 to about 5:1; about 1:2.5 to about 2.5:1.

In some embodiments, the ratio of the first glycoengineered polypeptide to the second glycoengineered polypeptide is about 1:1.5 to about 1.5:1.

In some embodiments, ratio of the first glycoengineered polypeptide to the third glycoengineered polypeptide is about 1:9, about 1:8, about 1:7, about 1:6, about 1:5, about 1:4, about 1:3, about 1:2.5, about 1:2, about 1:1.5, about 1:1, about 9:1, about 8:1, about 7:1, about 6:1, about 5:1, about 4:1, about 3:1, about 2.5:1 about, 2:1, or about 1.5:1.

In some embodiments, the ratio of the first glycoengineered polypeptide to the third glycoengineered polypeptide is about 1:5 to about 5:1; about 1:2.5 to about 2.5:1.

In some embodiments, the ratio of the first glycoengineered polypeptide to the third glycoengineered polypeptide is about 1:1.5 to about 1.5:1.

In some embodiments, ratio of the second glycoengineered polypeptide to the third glycoengineered polypeptide is about 1:9, about 1:8, about 1:7, about 1:6, about 1:5, about 1:4, about 1:3, about 1:2.5, about 1:2, about 1:1.5, about 1:1, about 9:1, about 8:1, about 7:1, about 6:1, about 5:1, about 4:1, about 3:1, about 2.5:1 about, 2:1, or about 1.5:1.

In some embodiments, the ratio of the second glycoengineered polypeptide to the third glycoengineered polypeptide is about 1:5 to about 5:1; about 1:2.5 to about 2.5:1.

In some embodiments, the ratio of the second glycoengineered polypeptide to the third glycoengineered polypeptide is about 1:1.5 to about 1.5:1.

In some embodiments, the first glycoengineered polypeptide is present at an amount of about 10-90% and the additional (e.g., second or third) glycoengineered polypeptide is present at an amount of about 90-10%.

In some embodiments, the first glycoengineered polypeptide is present at an amount of about 20-80% and the additional (e.g., second or third) glycoengineered polypeptide is present at an amount of about 80-20%.

In some embodiments, the first glycoengineered polypeptide is present at an amount of about 30-70% and the additional (e.g., second or third) glycoengineered polypeptide is present at an amount of about 70-30%.

In some embodiments, the first glycoengineered polypeptide is present at an amount of about 40-60% and the additional (e.g., second or third) glycoengineered polypeptide is present at an amount of about 60-40%.

In some embodiments, the first glycoengineered polypeptide is present at an amount of about 10% and the additional (e.g., second or third) glycoengineered polypeptide is present at an amount of about 90%.

In some embodiments, the first glycoengineered polypeptide is present at an amount of about 20% and the additional (e.g., second or third) glycoengineered polypeptide is present at an amount of about 80%.

In some embodiments, the first glycoengineered polypeptide is present at an amount of about 30% and the additional (e.g., second or third) glycoengineered polypeptide is present at an amount of about 70%.

In some embodiments, the first glycoengineered polypeptide is present at an amount of about 40% and the additional (e.g., second or third) glycoengineered polypeptide is present at an amount of about 60%.

In some embodiments, the first glycoengineered polypeptide is present at an amount of about 50% and the additional (e.g., second or third) glycoengineered polypeptide is present at an amount of about 50%.

In some embodiments, the first glycoengineered polypeptide is present at an amount of about 60% and the additional (e.g., second or third) glycoengineered polypeptide is present at an amount of about 40%.

In some embodiments, the first glycoengineered polypeptide is present at an amount of about 70% and the additional (e.g., second or third) glycoengineered polypeptide is present at an amount of about 30%.

In some embodiments, the first glycoengineered polypeptide is present at an amount of about 80% and the additional (e.g., second or third) glycoengineered polypeptide is present at an amount of about 20%.

In some embodiments, the first glycoengineered polypeptide is present at an amount of about 90% and the additional (e.g., second or third) glycoengineered polypeptide is present at an amount of about 10%.

In some embodiments, disclosed herein is a composition comprising a population of glycoengineered polypeptides disclosed herein, wherein the population of glycoengineered polypeptides has an N-glycan profile that is at least 30%, at least 40%, at least 50%, at least 60%, at least 70%, at least 80%, at least 90%, at least 95%, at least 98%, or about 100% homogeneous at one or more of the N-glycosylation site(s).

In some embodiments, the homogeneity of the N-glycan profile at one or more of the N-glycosylation sites is determined by N-glycan analysis, glycopeptide analysis or intact protein analysis.

In some embodiments, the N-glycan profile comprises about 30% to 40%, about 40% to about 50%, about 50% to about 60%, about 60% to about 70%, about 70% to about 80%, about 80% to about 90%, or about 90% to about 100% of the N-glycan of the structure provided herein.

In some embodiments, the population of glycoengineered polypeptides has an N-glycan profile comprising about 30% to about 40%, about 40% to about 50%, about 50% to about 60%, about 60% to about 70%, about 70% to about 80%, about 80% to about 90%, or about 90% to about 100% of the N-glycan of the structure provided herein among all glycans in the N-glycan profile.

In some embodiments, a glycoengineered polypeptide disclosed herein or a composition comprising the same may be useful to treat and/or prevent a disease described herein (e.g., a disease associated with increased and/or aberrant IgA), or to ameliorate a symptom associated with a disease, disorder or condition described herein.

The present disclosure also provides pharmaceutical compositions that, when administered to a subject (e.g., a human subject), e.g., when administered to a subject suffering from a disease associated with increased and/or aberrant IgA, deliver a glycoengineered polypeptide as described herein to such subject. Thus, in some embodiments, the present disclosure provides pharmaceutical compositions that comprise or deliver one or more glycoengineered polypeptides or one or more polynucleotides encoding such as described herein.

In some embodiments, a pharmaceutical composition is or comprises a composition according to the present disclosure.

Typically, a pharmaceutical composition includes an glycoengineered polypeptide as described herein, or a nucleic acid that encodes it in combination with one or more pharmaceutically acceptable carriers or excipients such as, for example one or more buffers, diluents, fillers, salts, solubilizers, stabilizers, and/or other materials as is known in the art. Those skilled in the art will be aware of a variety of carrier components appropriate to a particular active type (e.g., polypeptide versus nucleic acid, viral vector vs plasmid versus RNA, etc.) and/or route of administration (e.g., parenteral, enteral, etc.).

In some embodiments, a pharmaceutical composition, may comprise or deliver two or more different glycoengineered polypeptide, so that such agents may be administered in combination (e.g., substantially simultaneously or sequentially) to subject(s).

In some embodiments, a pharmaceutical composition may contain one or more agents that, for example, may improve stability of the composition and/or its active agent (e.g., to particular storage conditions and/or period(s) of time), facilitate delivery of the composition and/or its active agent, and/or otherwise enhance effectiveness (and/or reduce one or more undesirable side effects) of the active agent or composition once administered.

Alternatively or additionally, in some embodiments, a provided pharmaceutical composition may comprise or deliver another active agent in addition to a glycoengineered polypeptide as described herein.

Methods of Treatment and/or Prevention

The present disclosure, among other things, provides methods of treating and/or preventing a disease associated with increased and/or aberrant IgA (e.g., IgAN) in a subject comprising administering a composition as described herein, thereby improving at least one sign or symptom of disease associated with increased and/or aberrant IgA (e.g., IgAN)) in the subject after administration.

In some embodiments, provided herein are methods of treating and/or preventing a disease associated with increased and/or aberrant IgA (e.g., IgAN) comprising administering a composition as described herein.

Among other things, disclosed herein is the identification of glycoengineered polypeptides that specially bind to one or more target antibodies. In some embodiments, glycoengineered polypeptides disclosed herein have therapeutic value, e.g., in the treatment of disease associated with increased and/or aberrant IgA (e.g., IgAN).

A glycoengineered polypeptide that specifically binds to one or more target antibodies of the present disclosure can be used, inter alia, to treat, prevent, and/or improve disease associated with increased and/or aberrant IgA (e.g., IgAN).

Additionally or alternatively, a glycoengineered polypeptide that specifically binds to one or more target antibodies of the present disclosure can be used, inter alia, to treat, prevent, and/or improve, any number of diseases in which the target antibody levels are aberrantly high and/or in which a reduction of target antibody levels is sought.

A subject to be treated with methods described herein can be e.g., a patient having, or at risk of having, or is diagnosed as having disease associated with increased and/or aberrant IgA (e.g., IgAN).

Those skilled in the art, reading the present disclosure, will appreciate that provided compositions, may be useful for treating disease associated with increased and/or aberrant IgA (e.g., IgAN). In some embodiments, a subject disease associated with increased and/or aberrant IgA (e.g., IgAN) has or is characterized as having increased levels of target antibodies and/or immune complexes comprising the same, e.g., as compared to a healthy subject or to a subject who is not at risk of developing disease associated with increased and/or aberrant IgA (e.g., IgAN). In some embodiments, a subject having disease associated with increased and/or aberrant IgA (e.g., IgAN) has or is characterized as having aberrant target antibodies and/or immune complexes comprising the same, e.g., as compared to a healthy subject or to a subject who is not at risk of developing disease associated with increased and/or aberrant IgA (e.g., IgAN).

In some embodiments, a target antibody disclosed herein comprises an IgA1, or a fragment thereof, or an immune complex comprising the same.

In some embodiments, a target antibody disclosed herein comprises a gd-IgA1, or a fragment thereof or an immune complex comprising the same.

In some embodiments, a target antibody disclosed herein comprises an anti-gd-IgA1 autoantibody, or a fragment thereof or an immune complex comprising the same. In some embodiments, an anti-gd-IgA1 autoantibody is an IgG. In some embodiments, an anti-gd-IgA1 autoantibody is an IgM. In some embodiments, an anti-gd-IgA1 autoantibody is an IgE. In some embodiments, an anti-gd-IgA1 autoantibody is an IgD.

In some embodiments, a subject has increased levels of IgA1 or immune complexes comprising the same as compared to a subject who does not have a disease associated with increased and/or aberrant IgA, e.g., IgAN. In some embodiments, administration of a composition reduces a level IgA1 or immune complexes comprising the same as compared to a subject who has not been administered the pharmaceutical composition or as compared to the same subject prior to administration of the pharmaceutical composition. In some embodiments, a reduction in level of IgA1 or immune complexes comprising the same comprises degradation of IgA1 or immune complexes comprising the same. In some embodiments, a reduction in the level of IgA1 or immune complexes comprising the same is a result of internalization into a cell. In some embodiments, internalization comprises transporting to a lysosome and/or degradation. In some embodiments, administration of the pharmaceutical composition prevents IgA1 from binding to an antigen or a binding partner. In some embodiments, a binding partner of IgA1 comprises CD89, e.g., soluble CD89.

In some embodiments, a subject has increased levels of IgA1 or immune complexes comprising the same as compared to a subject who does not have a disease associated with increased and/or aberrant IgA, e.g., IgAN. In some embodiments, administration of the pharmaceutical composition prevents and/or reduces the formation of an immune complex comprising IgA1. In some embodiments, an immune complex disclosed herein comprises IgA1, an antigen recognized by IgA1, one or more components of a complement system and/or one or more additional immunoglobulins. In some embodiments, a complement component comprises: C3, C5b, C6, C7, C8, and/or C9, or fragments of any complement component or combinations thereof. In some embodiments, an immune complex comprises C3 or fragments of C3. In some embodiments, C3 fragments comprises iC3b, C3c, C3dg, or combinations thereof. In some embodiments, an immune complex comprises or more antibodies chosen from: an IgG, an IgA, an IgM, an IgD, an IgE, or fragments or combinations thereof.

In some embodiments, a subject has increased levels of IgA1 or immune complexes comprising the same as compared to a subject who does not have a disease associated with increased and/or aberrant IgA, e.g., IgAN. In some embodiments, administration of the pharmaceutical composition reduces activation of a complement system as compared to a subject who has not been administered the pharmaceutical composition or as compared to the same subject prior to administration of the pharmaceutical composition. In some embodiments, a complement system comprises: a lectin pathway, an alternative pathway, or a classical pathway, or a combination thereof.

In some embodiments, a subject has increased levels of IgA1 or immune complexes comprising the same as compared to a subject who does not have IgAN. In some embodiments, administration of the pharmaceutical composition prevents and/or reduces IgA deposits in the kidney of a subject as compared to a subject who has not been administered the pharmaceutical composition or as compared to the same subject prior to administration of the pharmaceutical composition.

In some embodiments, a subject has detectable levels of gd-IgA1 or immune complexes comprising the same as compared to a subject who does not have a disease associated with increased and/or aberrant IgA, e.g., IgAN. In some embodiments, administration of a composition reduces a level gd-IgA1 or immune complexes comprising the same as compared to a subject who has not been administered the pharmaceutical composition or as compared to the same subject prior to administration of the pharmaceutical composition. In some embodiments, a reduction in level of gd-IgA1 or immune complexes comprising the same comprises degradation of gd-IgA1 or immune complexes comprising the same. In some embodiments, a reduction in the level of gd-IgA1 or immune complexes comprising the same is a result of internalization into a cell. In some embodiments, internalization comprises transporting to a lysosome and/or degradation.

In some embodiments, a subject has increased levels of gd-IgA1 or immune complexes comprising the same as compared to a subject who does not have a disease associated with increased and/or aberrant IgA, e.g., IgAN. In some embodiments, administration of the pharmaceutical composition prevents and/or reduces the formation of an immune complex comprising gd-IgA1. In some embodiments, an immune complex disclosed herein comprises gd-IgA1, an antigen recognized by gd-IgA1, one or more components of a complement system and/or one or more additional immunoglobulins. In some embodiments, a complement component comprises: C3, C5b, C6, C7, C8, and/or C9, or fragments of any complement component or combinations thereof. In some embodiments, an immune complex comprises C3 or fragments of C3. In some embodiments, C3 fragments comprises iC3b, C3c, C3dg, or combinations thereof. In some embodiments, an immune complex comprises or more antibodies chosen from: an IgG, an IgA, an IgM, an IgD, an IgE, or fragments or combinations thereof.

In some embodiments, a subject has increased levels of gd-IgA1 or immune complexes comprising the same as compared to a subject who does not have a disease associated with increased and/or aberrant IgA, e.g., IgAN. In some embodiments, administration of the pharmaceutical composition reduces activation of a complement system as compared to a subject who has not been administered the pharmaceutical composition or as compared to the same subject prior to administration of the pharmaceutical composition. In some embodiments, a complement system comprises: a lectin pathway, an alternative pathway, or a classical pathway, or a combination thereof.

In some embodiments, a subject has increased levels of gd-IgA1 or immune complexes comprising the same as compared to a subject who does not have a disease associated with increased and/or aberrant IgA, e.g., IgAN. In some embodiments, administration of the pharmaceutical composition prevents and/or reduces gd-IgA1 deposits in the kidney of a subject as compared to a subject who has not been administered the pharmaceutical composition or as compared to the same subject prior to administration of the pharmaceutical composition.

In some embodiments, a subject has detectable levels of anti-gd-IgA1 or immune complexes comprising the same as compared to a subject who does not have a disease associated with increased and/or aberrant IgA, e.g., IgAN. In some embodiments, administration of a composition reduces a level anti-gd-IgA1 or immune complexes comprising the same as compared to a subject who has not been administered the pharmaceutical composition or as compared to the same subject prior to administration of the pharmaceutical composition. In some embodiments, a reduction in level of anti-gd-IgA1 or immune complexes comprising the same comprises degradation of gd-IgA1 or immune complexes comprising the same. In some embodiments, a reduction in the level of anti-gd-IgA1 or immune complexes comprising the same is a result of internalization into a cell. In some embodiments, internalization comprises transporting to a lysosome and/or degradation.

In some embodiments, a subject has increased levels of anti-gd-IgA1 or immune complexes comprising the same as compared to a subject who does not have a disease associated with increased and/or aberrant IgA, e.g., IgAN. In some embodiments, administration of the pharmaceutical composition prevents and/or reduces the formation of an immune complex comprising anti-gd-IgA1. In some embodiments, an immune complex disclosed herein comprises anti-gd-IgA1, an antigen recognized by anti-gd-IgA1, one or more components of a complement system and/or one or more additional immunoglobulins. In some embodiments, a complement component comprises: C3, C5b, C6, C7, C8, and/or C9, or fragments of any complement component or combinations thereof. In some embodiments, an immune complex comprises C3 or fragments of C3. In some embodiments, C3 fragments comprises iC3b, C3c, C3dg, or combinations thereof. In some embodiments, an immune complex comprises or more antibodies chosen from: an IgG, an IgA, an IgM, an IgD, an IgE, or fragments or combinations thereof.

In some embodiments, a subject has increased levels of anti-gd-IgA1 or immune complexes comprising the same as compared to a subject who does not have a disease associated with increased and/or aberrant IgA, e.g., IgAN. In some embodiments, administration of the pharmaceutical composition reduces activation of a complement system as compared to a subject who has not been administered the pharmaceutical composition or as compared to the same subject prior to administration of the pharmaceutical composition. In some embodiments, a complement system comprises: a lectin pathway, an alternative pathway, or a classical pathway, or a combination thereof.

In some embodiments, a subject has increased levels of anti-gd-IgA1 or immune complexes comprising the same as compared to a subject who does not have a disease associated with increased and/or aberrant IgA, e.g., IgAN. In some embodiments, administration of the pharmaceutical composition prevents and/or reduces anti-gd-IgA1 deposits in the kidney of a subject as compared to a subject who has not been administered the pharmaceutical composition or as compared to the same subject prior to administration of the pharmaceutical composition.

Additionally or alternatively, a glycoengineered polypeptide that specifically binds to one or more target antibodies of the present disclosure (or a composition comprising the same) can be used, to inhibit elevated production of gd-IgA1. In some embodiments, administration of a glycoengineered polypeptide disclosed herein to a cell, tissue, or subject reduces production of gd-IgA1. In some embodiments, administration of a glycoengineered polypeptide disclosed herein to a cell, tissue, or subject modulates enzyme expression in IgA1-producing cells, e.g., such that the IgA1 expressed is not gd-IgA1. In some embodiments, the IgA1 expressed has a glycan profile that is similar to a reference glycan profile. In some embodiments, a reference glycan profile is an IgA1 glycan profile produced in a healthy individual.

In some embodiments, administration of a glycoengineered polypeptide disclosed herein to a subject reduces the number of cells secreting gd-IgA1.

Additionally or alternatively, a glycoengineered polypeptide that specifically binds to one or more target antibodies of the present disclosure (or a composition comprising the same) can be used to modulate production of anti-gd-IgA1 autoantibodies. In some embodiments, administration of a glycoengineered polypeptide disclosed herein to a cell, tissue, or subject reduces production of anti-gd-IgA1 autoantibodies. In some embodiments, administration of a glycoengineered polypeptide disclosed herein to a cell, tissue, or subject depletes cells producing anti-gd-IgA1 autoantibodies. In some embodiments, administration of a glycoengineered polypeptide disclosed herein to a cell, tissue, or subject allows for the manipulation of affinity maturation of anti-gd-IgA1 autoantibodies to reduce affinity for the autoantigen (gd-IgA1). In some embodiments, administration of a glycoengineered polypeptide disclosed herein to a cell, tissue, or subject removes anti-gd-IgA1 autoantibodies from circulation.

Additionally or alternatively, a glycoengineered polypeptide that specifically binds to one or more target antibodies of the present disclosure (or a composition comprising the same) can be used, to inhibit formation of pathogenic IgA1-containing immune complexes. In some embodiments, administration of a glycoengineered polypeptide disclosed herein to a cell, tissue, or subject prevents and/or inhibits immune complex formation and/or enhances removal of immune complexes from circulation and/or promotes catabolism of immune complexes. In some embodiments, administration of a glycoengineered polypeptide disclosed herein to a cell, tissue, or subject blocks epitopes of autoantigen (Gd-IgA1) by non-crosslinking antibodies. In some embodiments, administration of a glycoengineered polypeptide disclosed herein to a cell, tissue, or subject inhibits anti-gd-IgA1 autoantibodies from binding to the antigen with an epitope-containing a glycopeptide or glycomimetic. In some embodiments, administration of a glycoengineered polypeptide disclosed herein to a cell, tissue, or subject prevents and/or inhibits activation of complement.

Additionally or alternatively, a glycoengineered polypeptide that specifically binds to one or more target antibodies of the present disclosure (or a composition comprising the same) can be used, to prevent glomerular deposition and/or injury. In some embodiments, administration of a glycoengineered polypeptide disclosed herein to a cell, tissue, or subject inhibits activation of mesangial cells. In some embodiments, administration of a glycoengineered polypeptide disclosed herein to a cell, tissue, or subject reduces complement activation in situ. In some embodiments, administration of a glycoengineered polypeptide disclosed herein to a cell, tissue, or subject prevents and/or inhibits binding of IgA1-containing immune complexes to mesangial cells. In some embodiments, administration of a glycoengineered polypeptide disclosed herein to a cell, tissue, or subject prevents and/or inhibits mesangial-cell signaling induced by IgA1-containing immune complexes.

### Administration

In some embodiments, of the disclosure, provided are methods comprising administering to a subject a composition according to the present disclosure. In some embodiments, a method comprises administering to a subject a pharmaceutical composition comprising a glycoengineered polypeptide according to the present disclosure.

Delivery of a glycoengineered polypeptide can be achieved e.g., by administration of a pharmaceutical composition as described herein, such as a pharmaceutical composition that comprises a glycoengineered polypeptide or a nucleic acid that encodes it, for example via oral ingestion, inhalation, topical application or parenteral administration (e.g., cutaneous, subcutaneous, intraperitoneal, intramuscular or intravenous injection). In some embodiments, administration is by intravenous or intramuscular injection. In some embodiments, local administration may be or comprise topical administration (e.g., to the skin) or parenteral administration (e.g., by injection to a site of deposition such as to the kidney).

In some embodiments, delivery of a glycoengineered polypeptide can be achieved e.g., by administration of a pharmaceutical composition as described herein, such as a pharmaceutical composition that comprises a glycoengineered polypeptide or a nucleic acid that encodes it, may be oral, rectal, ophthalmic (including intravitreal or intracameral), nasal, topical (including buccal and sublingual), intrauterine, vaginal or parenteral (including subcutaneous, intraperitoneal, intramuscular, intravenous, intradermal, intracranial, intratracheal, and epidural). Those skilled in the art will be aware of typical guiding principles for formulation of pharmaceutical compositions for administration by such routes. For example, such techniques may include the step of bringing into association a glycoengineered polypeptide or a nucleic acid that encodes it and the pharmaceutical carrier(s) or excipient(s). In some embodiments, compositions are prepared by uniformly and intimately bringing into association the active ingredient with liquid carriers or finely divided solid carriers or both, and then, if necessary, shaping the product.

In some embodiments, the administration step comprises intravenous injection, intraperitoneal injection, subcutaneous injection, transdermal injection, or intramuscular injection.

Among other things, disclosed herein is the identification of glycoengineered polypeptides and compositions comprising the same that have therapeutic value, e.g., in the treatment of a disease associated with increased and/or aberrant IgA (e.g., IgAN).

In some embodiments, glycoengineered polypeptides and compositions comprising the same are delivered to (e.g., by administration of a pharmaceutical composition as described herein that comprises or delivers such agent, or a nucleic acid that encodes it) to a subject suffering from or susceptible to a disease associated with increased and/or aberrant IgA as described herein.

In some embodiments, the subject is a human.

In some embodiments, the subject has detectable levels of one or more target antibodies, e.g., IgA1, gd-IgA1, and/or anti-gd-IgA1.

In some embodiments, a composition according to the present disclosure are delivered to a subject suffering from or susceptible to disease associated with increased and/or aberrant IgA (e.g., IgAN).

In some embodiments, the subject has or is diagnosed as having a disease associated with increased and/or aberrant IgA (e.g., IgAN). In some embodiments, the subject is a human.

In some embodiments, administration of a composition according to the present disclosure alleviates one or more symptoms of disease associated with increased and/or aberrant IgA (e.g., IgAN).

In some embodiments, administration of a glycoengineered polypeptide that binds to an IgA1 or a fragment or a complex thereof treats and/or prevents disease associated with increased and/or aberrant IgA (e.g., IgAN).

In some embodiments, administration of a glycoengineered polypeptide that binds to a gd-IgA1 or a fragment or a complex thereof treats and/or prevents disease associated with increased and/or aberrant IgA (e.g., IgAN).

In some embodiments, administration of a glycoengineered polypeptide that binds to a anti-gd-IgA1 or a fragment or a complex thereof treats and/or prevents disease associated with increased and/or aberrant IgA (e.g., IgAN).

In some embodiments, a method disclosed herein is a treatment method.

In some embodiments, a method disclosed herein is a prevention method.

### Methods of Assessing Target Antibody Levels

Also disclosed herein are methods of assessing a level assessing a level of a target antibody in a sample from a subject, and administering a pharmaceutical composition comprising a glycoengineered polypeptide disclosed herein if the level of the target antibody is higher than a comparator. In some embodiments, a comparator comprises a predetermined reference sample such as a sample obtained from an otherwise similar subject who does not have a disease or disorder, or a symptom of a disease or disorder.

In some embodiments, disease or disorder is IgA nephropathy.

In some embodiments, disease or disorder is dermatitis herpetiformis.

In some embodiments, disease or disorder is Henoch-Schoenlein purpura

In some embodiments, a level of target antibody is assessed using an assay that detects the level and/or activity of IgA1, gd-IgA1, or anti-gd-IgA1, or a combination thereof.

In some embodiments, the assay is a dot blot assay.

In some embodiments, the assay is a capture ELISA.

Exemplary assays that can be used to detect the level of an anti-gd-IgA1 or immune complexes comprising the same are disclosed in U.S. Pat. No. 9,655,963, the entire contents of which are hereby incorporated by reference.

Among other things, the present disclosure provides the insight that an assay such as an assay disclosed in U.S. Pat. No. 9,655,963 which detects anti-gd-IgA1 or immune complexes comprising the same can be used to identify a subject having or at risk of developing a disease associated with increased and/or aberrant IgA, e.g., IgAN. In some embodiments, a subject identified as having or at risk of developing a disease associated with increased and/or aberrant IgA, e.g., IgAN, has increased levels of an anti-gd-IgA1 or immune complexes comprising the same compared to a healthy subject. In some embodiments, a subject identified as having or at risk of developing a disease associated with increased and/or aberrant IgA, e.g., IgAN, can benefit from the administration of a glycoengineered polypeptide disclosed herein.

Additional assays that can be used to detect the level of an anti-gd-IgA1 or immune complexes comprising the same are disclosed are disclosed in Suzuki H. et al., (2009) J Clin Investigation 119:6, the entire contents of which are hereby incorporated by reference. Suzuki teaches a dot-blot assay for detecting glycan-specific IgG antibodies in IgAN patients. The assay disclosed in Suzuki can differentiate IgAN patients form healthy controls with high specificity and sensitivity.

Among other things, the present disclosure provides the insight that an assay such as an assay disclosed in Suzuki et al., 2009, which detects anti-gd-IgA1 or immune complexes comprising the same can be used to identify a subject having or at risk of developing a disease associated with increased and/or aberrant IgA, e.g., IgAN. In some embodiments, a subject identified as having or at risk of developing a disease associated with increased and/or aberrant IgA, e.g., IgAN, has increased levels of an anti-gd-IgA1 or immune complexes comprising the same compared to a healthy subject. In some embodiments, a subject identified as having or at risk of developing a disease associated with increased and/or aberrant IgA, e.g., IgAN, can benefit from the administration of a glycoengineered polypeptide disclosed herein.

### Dosing Regimens

In some embodiments, a method comprises administering a composition once. In some embodiments, a method comprises administering a composition repeatedly.

In some embodiments, administration of a composition is continued to maintain remission (e.g., keep target antibodies and/or immune complexes comprising the same at a low and/or undetectable level) and/or avoid relapse.

Amounts glycoengineered polypeptide administered in a single dose may depend on the nature and/or severity of the condition being treated and/or on the nature of prior treatments that the patient has undergone. In some embodiments, the attending physician decides the amount of glycoengineered polypeptide with which to treat each individual patient. In some embodiments, the attending physician initially administers low doses of glycoengineered polypeptides of the present invention and observe the patient's response. In some embodiments, larger doses are administered until an optimal therapeutic effect is obtained for the patient, after which dosage is not increased further.

In some embodiments, a glycoengineered polypeptide according to the present disclosure is delivered in an amount effective to reduce levels of one or more target antibodies or a fragment or an immune complex comprising the same.

In some embodiments, a glycoengineered polypeptide according to the present disclosure is delivered in an amount effective to reduce levels of an IgA1 antibody or a fragment or an immune complex comprising the same.

In some embodiments, a glycoengineered polypeptide according to the present disclosure is delivered in an amount effective to reduce levels of a gd-IgA1 antibody or a fragment or an immune complex comprising the same.

In some embodiments, a glycoengineered polypeptide according to the present disclosure is delivered in an amount effective to reduce levels of an anti-gd-IgA1 autoantibody or a fragment or an immune complex comprising the same.

### Combination Therapies

According to the present disclosure, glycoengineered polypeptides may be administered in combination with one or more therapies such as standard of care that is typically used for the treatment and/or management of a disease associated with increased and/or aberrant IgA, e.g., IgAN.

In some embodiments, glycoengineered polypeptides may be administered in combination with one or more pharmaceutical agents. For example, a glycoengineered polypeptide may be administered in combination with one or more therapeutic agents for a disease associated with increased and/or aberrant IgA, e.g., IgAN (such as agents that ameliorate symptoms of a disease associated with increased and/or aberrant IgA, e.g., IgAN), and/or in combination with one or more other pharmaceutical agents. In some embodiments, glycoengineered polypeptides may be administered in combination with one or more therapies and/or agents that are prescribed by a clinician for treatment of a disease associated with increased and/or aberrant IgA, e.g., IgAN.

In some embodiments, a pharmaceutical composition according to the present disclosure is administered in combination with one or more additional therapies. In some embodiments, a pharmaceutical composition according to the present disclosure is administered in combination with a blood pressure medication, a proteinuria management therapy, a renal protectant (e.g., a SGLT2 inhibitor), glucocorticoids, or a combination thereof. In some embodiments, a pharmaceutical composition according to the present disclosure is administered in combination with one or more therapies and/or agents that are prescribed by a clinician for treatment a disease associated with increased and/or aberrant IgA, e.g., IgAN.

In some embodiments, a pharmaceutical composition according to the present disclosure is administered in combination with blood pressure medication, e.g., as is typically used to manage high blood pressure.

In some embodiments, a pharmaceutical composition according to the present disclosure is administered in combination with therapies used for management of proteinuria.

In some embodiments, a pharmaceutical composition according to the present disclosure is administered in combination with a renal protectant, e.g., as used in non-diabetic kidney disease. In some embodiments, a pharmaceutical composition according to the present disclosure is administered in combination with an SGLT2 inhibitor.

In some embodiments, a pharmaceutical composition according to the present disclosure is administered in combination with glucocorticoids.

In some embodiments, a pharmaceutical composition according to the present disclosure is administered in combination with administration of intravenous immunoglobulin compositions (IVIGs).

### Characterization of Glycoengineered Polypeptides

The present disclosure provides, among other things, glycoengineered polypeptides that specifically bind to target antibodies and thereby causing degradation of target antibodies and/or immune complexes comprising the same. In some embodiments, target antibodies degradation comprises internalized into a cell for degradation (e.g., by transporting the target antibodies to a lysosome). In some embodiments, glycoengineered polypeptides specifically binding to target antibodies according to the present disclosure are characterized in that they inhibit one or more pathological functions of target antibodies including their ability to form immune complexes and/or activate complement pathways.

In some embodiments, a glycoengineered polypeptide according to the present disclosure is used to degrade and/or reduce the levels of target antibodies and/or immune complexes comprising the same. In some embodiments, a glycoengineered polypeptide is used to lower target antibodies levels, such as lowering elevated plasma target antibodies levels in a subject with a disease associated with increased and/or aberrant IgA, e.g., IgAN. In some embodiments, a glycoengineered polypeptide is used to reduce and/or remove immune complexes comprising one or more target antibodies in a subject with a disease associated with increased and/or aberrant IgA, e.g., IgAN.

In some embodiments, the present disclosure provides glycoengineered polypeptides characterized in that when administered to a cell, tissue, or subject, the glycoengineered polypeptide which is bound to a target antibody via the first moiety and to an endocytic receptor via a second moiety results in degradation of the target antibody.

In some embodiments, degradation comprises internalization into a cell. In some embodiments, degradation comprises lysosomal degradation. In some embodiments, degradation occurs in a liver cell.

In some embodiments, a target antibody comprises an immune complex comprising the same. In some embodiments, an immune complex disclosed herein comprises one or more antibodies, an antigen recognized by one or more antibodies, and/or one or more components of a complement system. In some embodiments, a complement component comprises: C3, C5b, C6, C7, C8, and/or C9, or fragments of any complement component or combinations thereof. In some embodiments, an immune complex comprises C3 or fragments of C3. In some embodiments, C3 fragments comprises iC3b, C3c, C3dg, or combinations thereof. In some embodiments, an immune complex comprises or more antibodies chosen from: an IgG, an IgA, an IgM, an IgD, an IgE, or fragments or combinations thereof.

In some embodiments, a target antibody is an IgA1 antibody or a fragment thereof or an immune complex comprising the same.

In some embodiments, a target is a gd-IgA1 antibody or a fragment thereof or an immune complex comprising the same.

In some embodiments, a target is an gd-IgA1 autoantibody or a fragment thereof or an immune complex comprising the same.

In some embodiments, the present disclosure provides glycoengineered polypeptides characterized in that when administered to a cell, tissue, or subject, the glycoengineered polypeptide which is bound to a target antibody via the first moiety prevents activation of a complement pathway or component thereof. In some embodiments, a complement pathway comprises a classical pathway, an alternative pathway, or a lectin pathway.

In some embodiments, the present disclosure provides glycoengineered polypeptides characterized in that when administered to a cell, tissue, or subject, the glycoengineered polypeptide which is bound to a target antibody via the first moiety prevents proteinuria, decreased albumin levels and/or edema.

In some embodiments, the present disclosure provides glycoengineered polypeptides characterized in that when administered to a cell, tissue, or subject, the glycoengineered polypeptide which is bound to a target antibody via the first moiety prevents and/or reduces formation of an immune complex comprising a target antibody.

In some embodiments, the present disclosure provides glycoengineered polypeptides characterized in that when administered to a cell, tissue, or subject, the glycoengineered polypeptide which is bound to a target antibody via the first moiety prevents and/or reduces IgA deposits in one or more tissues or organs. In some embodiments, the tissue is a renal tissue. In some embodiments, the organ is a kidney.

### Method of Making Glycoengineered Polypeptides

The present disclosure, among other things, provides methods of making a glycoengineered polypeptide comprising a first moiety comprising one or more peptides that specifically binds to a target (e.g., a target antibody or a fragment or a complex thereof) and a second moiety comprising one or more glycans conjugated to the first moiety.

A glycoengineered polypeptide disclosed herein can be made using methods disclosed in U.S. Provisional Patent Application No. 63/410,955 filed on Sep. 28, 2022 and U.S. Provisional Patent Application No. 63/410,936, filed on Sep. 28, 2022, and International Patent Application PCT/EP2023/076767 filed on Sep. 27, 2023, the entire contents of each of which are hereby incorporated by reference.

For example, in U.S. 63/410,955, Section 5.3 discloses Leishmania host cells; Section 5.4 discloses exemplary methods of genetically engineering a Leishmania cell for expressing glycoengineered polypeptides, Section 5.5 disclose exemplary methods of culturing Leishmania host cells; and Section 5.6 discloses exemplary uses of Leishmania host cells as an expression system.

As another example, in International Patent Application PCT/EP2023/076767, Section 7.1 discloses Leishmania host cells including modifications that can be made to a Leishmania host cell for producing glycoengineered polypeptides, Section 7.2 disclose methods of genetically engineering Leishmania host cells for producing glycoengineered polypeptides, and Section 7.3 discloses methods of culturing Leishmania host cells. An exemplary method of making glycoengineered polypeptides using Leishmania host cells is provided in Example 1 herein.

In particular, as disclosed in PCT/EP2023/076767, exemplary Leishmania strains that can be used to make glycoengineered polypeptides disclosed herein include: StCGP3558, StCGP4564, StCGP5359, or StCGP5942.

As would be understood by persons with ordinary skill in the art, such methods and host cells can also be used for making glycoengineered polypeptides disclosed herein.

In some embodiments, the one or more glycans of the second moiety are conjugated to the first moiety at one or more glycosylation sites with in vivo glycosylation, e.g., in a cell. In some embodiments, a cell is a Leishmania host cell. In some embodiments, a cell is a glycoengineered yeast host cell, e.g., glycoengineered Pichia pastoris host cell.

In some embodiments, the one or more glycans of the second moiety are conjugated to the first moiety at one or more glycosylation sites with chemical conjugation, e.g., using Click chemistry.

Also disclosed herein are methods for making a glycoengineered polypeptide. In one embodiment, provided herein is a method of producing a glycoengineered polypeptide in vivo, using a Leishmania host cell described herein. In some embodiments, provided herein is a method for producing a glycoengineered polypeptide, said method comprising (i) culturing a Leishmania host cell under conditions suitable for polypeptide production and (ii) isolating said glycoengineered polypeptide. In a specific embodiment, the Leishmania host cell comprises: (a) a recombinant nucleic acid encoding a glycoengineered polypeptide; and (b) a recombinant nucleic acid encoding one or more recombinant N-acetylgalactosamine (GalNAc) transferases. In certain embodiments, the Leishmania host cell is capable of producing glycoengineered polypeptide comprising a biantennary, GalNAc-terminated N-glycan. In particular, the Leishmania host cells provided herein is capable of producing glycoengineered polypeptide comprising an N-glycan of the following structure:

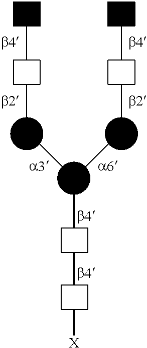

wherein the black square represents an N-acetyl galactosamine (GalNAc), the white square represents an N-acetylglucosamine (GlcNAc) residue and the black circle represents a mannose (Man) residue, and wherein X represents an amino acid residue of the glycoengineered polypeptide.

In certain embodiments, the glycoengineered polypeptide produced by the Leishmania host cell is a therapeutic polypeptide, i.e., a polypeptide used in the treatment of a disease or disorder. For example, the glycoengineered polypeptide produced by the Leishmania host cell can be peptide or an antibody.

### Leishmania Host Cells

Provided herein are Leishmania host cells for the production of glycoengineered polypeptides disclosed herein, or a population of glycoengineered polypeptides, wherein the Leishmania host cells comprise: (a) a recombinant nucleic acid encoding a glycoengineered polypeptide disclosed herein; and (b) a recombinant nucleic acid encoding one or more recombinant N-acetylgalactosamine (GalNAc) transferases. In certain embodiments, the Leishmania host cells provided herein are capable of producing glycoengineered polypeptides comprising a biantennary, GalNAc-terminated N-glycan. In particular, the Leishmania host cells provided herein are capable of producing glycoengineered polypeptides comprising an N-glycan of the following structure:

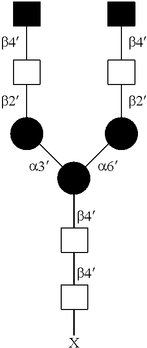

wherein the black square represents an N-acetyl galactosamine (GalNAc), the white square represents an N-acetylglucosamine (GlcNAc) residue and the black circle represents a mannose (Man) residue, and wherein X represents an amino acid residue of the glycoengineered polypeptide.

In certain embodiments, the Leishmania host cells provided herein comprise a recombinant nucleic acid encoding one or more recombinant N-acetylgalactosamine (GalNAc) transferases disclosed herein. In certain embodiments, the Leishmania host cells provided herein comprise a recombinant nucleic acid encoding one or more additional recombinant glycosyltransferases disclosed herein. In certain embodiments, one or more endogenous enzymes disclosed herein from the glycan biosynthesis pathway of the Leishmania host cells provided herein have been deleted, mutated and/or functionally inactivated. In certain embodiments, the Leishmania host cells provided herein further comprise a recombinant nucleic acid encoding heterologous UDP-GalNAc biosynthetic pathway proteins capable of generating UDP-GalNAc. In certain embodiments, the Leishmania host cells provided herein comprise a recombinant nucleic acid encoding a heterologous UDP-GalNAc transporter protein capable of transporting UDP-GalNAc to the secretory pathway.

In certain embodiments, the Leishmania host cells provided herein have been genetically engineered such that the formation of an O-linked GlcNAc on a polypeptide produced in the Leishmania host cell is reduced or eliminated. Leishmania host cells that have been genetically engineered to reduce or eliminate the formation of an O-linked GlcNAc are described, for example, in WO 2021/140143, which is incorporated herein by reference in its entirety. x

In certain embodiments, the Leishmania host cells provided herein below are genetically engineered using the methods described herein. In certain embodiments, the Leishmania host cells provided herein below are cultured according to the methods described herein.

Other suitable host cells comprise liver cells, myeloid cells, immune cells, endothelial cells, parenchymal cells or epithelial cells. In some embodiments, the immune cell is a dendritic cell, a macrophage, a monocyte, a microglia cell, a granulocyte or a B lymphocyte.

### Methods of Culturing Leishmania Host Cells

Provided herein are methods for culturing Leishmania host cells. In one embodiment, the Leishmania host cells are cultured using any of the standard culturing techniques known in the art. For example, cells are routinely grown in rich media like Brain Heart Infusion, Trypticase Soy Broth or Yeast Extract, all containing 5 μg/ml Hemin. Additionally, incubation is done at 26° C. in the dark as static or shaking cultures for 2-3 days. In some embodiments, cultures of recombinant cell lines contain the appropriate selective agents. Non-limiting exemplary selective agents are provided in Table 1.

TABLE 1
Selective agents used during transfection (50% concentration for
preselection and 100% concentration for main selection) and standard
culturing of L. tarentolae. Double amounts of the selective agents
could be used if higher selection pressure was intended.

| | | Concentration | | | |
|---|---|---|---|---|---|
| | Resistance | (100%) | | Concentration | |
| Selective | conferring | main selection/ | | (50%) | |
| agent | gene | standard culturing | | preselection | |
| Nourseothricin | sat | 50 | μg/ml | 25 | μg/ml |
| Geneticin | neo | 50 | μg/ml | 25 | μg/ml |
| Paromomycin | neo | 300 | μg/ml | 150 | μg/ml |
| Zeocin | ble | 150 | μg/ml | 75 | μg/ml |
| Hygromycin | hyg | 50 | μg/ml | 25 | μg/ml |
| Blasticidin | bsd | 5 | μg/ml | 2.5 | μg/ml |
| Puromycin | pac | 5 | μg/ml | 2.5 | μg/ml |

In certain embodiments, the Leishmania host cells are cultured in a growth medium comprising GalNAc. In certain embodiments, the growth medium comprises at least 1 mM, at least 2 mM, at least 3 mM, at least 4 mM, at least 5 mM, at least 6 mM, at least 7 mM, at least 8 mM, at least 9 mM, at least 10 mM, at least 11 mM, at least 12 mM, at least 13 mM, at least 14 mM, at least 15 mM, at least 16 mM, at least 17 mM, at least 18 mM, at least 19 mM, or at least 20 mM GalNAc. In certain embodiments, the growth medium comprises about 1 mM to about 5 mM, about 5 mM to about 10 mM, about 10 mM to about 15 mM, or about 15 mM to about 20 mM GalNAc. In certain embodiments, the growth medium comprises about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM GalNAc. In certain embodiments, the growth medium comprises about 10 mM GalNAc.

In certain embodiments, the Leishmania host cells are cultured in a growth medium comprising GlcNAc. In certain embodiments, the growth medium comprises at least 1 mM, at least 2 mM, at least 3 mM, at least 4 mM, at least 5 mM, at least 6 mM, at least 7 mM, at least 8 mM, at least 9 mM, at least 10 mM, at least 11 mM, at least 12 mM, at least 13 mM, at least 14 mM, at least 15 mM, at least 16 mM, at least 17 mM, at least 18 mM, at least 19 mM, or at least 20 mM GlcNAc. In certain embodiments, the growth medium comprises about 1 mM to about 5 mM, about 5 mM to about 10 mM, about 10 mM to about 15 mM, or about 15 mM to about 20 mM GlcNAc. In certain embodiments, the growth medium comprises about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM GlcNAc.

In certain embodiments, a Leishmania host cell may be used as an expression system for making a glycoengineered polypeptide disclosed herein or a population of glycoengineered polypeptides. In certain embodiments, the glycoengineered polypeptide degrader may be a heterologous, non-Leishmania protein, such as a therapeutic protein (e.g., an antibody). Other methods of producing Leishmania host cells for use as expression systems are known and may also be used, for example, see WO 2019/002512, WO 2021/140144 and WO 2021/140143, each of which are incorporated herein by reference in their entirety. Use of Leishmania host cells to make monoclonal antibodies are also known. Exemplary methods are described in WO 2022/053673, which is incorporated herein by reference in its entirety.

The compositions comprising the Leishmania host cells can comprise additional components suitable for maintenance and survival of the Leishmania host cells, and can additionally comprise additional components required or beneficial to the production of glycoengineered bifunctional degraders by the Leishmania host cells, e.g., inducers for inducible promoters, such as arabinose, IPTG.

### Yeast or Filamentous Fungal Host Cells

Provided herein are yeast or filamentous fungal host cells for the production of glycoengineered polypeptides disclosed herein, or a population of glycoengineered polypeptides. In some embodiments, a yeast or filamentous fungal host cell is a K. lactis host cells. In some embodiments, a yeast or filamentous fungal host cell is a Pichia pastoris host cell. In some embodiments, a yeast or filamentous fungal host cell is a Pichia methanolica host cell. In some embodiments, a yeast or filamentous fungal host cell is a Hansenula host cell.

Exemplary yeast or filamentous fungal host cells that can be used to produce glycoengineered polypeptides disclosed herein are disclosed in U.S. Pat. No. 8,206,949, the entire contents of which are hereby incorporated by reference.

Exemplary yeast or filamentous fungal host cells that can be used to produce glycoengineered polypeptides disclosed herein are disclosed in U.S. Pat. No. 7,981,660, the entire contents of which are hereby incorporated by reference.

Exemplary yeast or filamentous fungal host cells that can be used to produce glycoengineered polypeptides disclosed herein are disclosed in U.S. Pat. No. 8,883,483, the entire contents of which are hereby incorporated by reference.

In some embodiments, a yeast or filamentous fungal host cell has been genetically modified to produce glycoproteins with a predominant N-glycan glycoform.

In certain embodiments, the yeast or filamentous fungal host cells provided herein are capable of producing glycoengineered polypeptides comprising a biantennary, GalNAc-terminated N-glycan. In particular, the yeast or filamentous fungal host cells provided herein are capable of producing glycoengineered polypeptides comprising an N-glycan of the following structure:

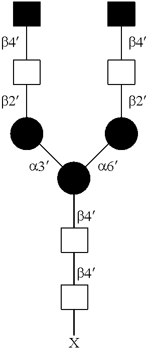

wherein the black square represents an N-acetyl galactosamine (GalNAc), the white square represents an N-acetylglucosamine (GlcNAc) residue and the black circle represents a mannose (Man) residue, and wherein X represents an amino acid residue of the glycoengineered polypeptide.

### EXAMPLES

### Example 1: Construction of a Glycoengineered Polypeptide of CD89

This example describes the construction and properties of an exemplary glycoengineered polypeptide of CD89 for use in binding to IgA antibodies and complexes comprising the same.

The Immunoglobulin alpha Fc receptor FcαRI, or CD89, is a type I transmembrane glycoprotein expressed at the surface of myeloid cells and member of the Ig superfamily. CD89 binds both IgA1 and IgA2 with similar affinity (Ka˜106 M-1). The site of interaction between CD89 and IgA was identified in the first extracellular domain of CD89 and the Cα2/Cα3 junction of IgA. (van der Boog et al; J Immunol 1 Feb. 2002; 168 (3): 1252-1258, herein “van der Boog 2002”). Soluble CD89 (sCD89) with different glycosylation has been shown to have an effect on CD89's binding affinity to IgA (Göritzer, K. et al. J. Biol. Chem. 294, 13995-14008 (2019), herein “Göritzer 2009”). It was demonstrated in Göritzer 2009 that, the shorter the glycan, the better the affinity, with GlcNAc providing the best affinity to IgA. This data also predicts a 1:1 binding model rather than 2:1 as listed in most literature and in crystal structures.

Methods: The construction of the glycoengineered polypeptide of soluble CD89 (sCD89) comprised three major steps. First, to express sCD89 in Leishmania tarentolae, the native signal peptide of CD89 (amino acid 1-21) was replaced with a Leishmania derived signal sequence (MIASSVRHAVILLLVAVAMMGGVIA; SEQ ID NO: 42) on the CD89 sequence, which spanned from amino acid 22 to amino acid 216 (numbering according to Uniprot P24071). While a Leishmania signal peptide was used in this Example, other suitable signal peptides as can be readily envisioned by an ordinarily skilled artisan can also be used. Second, the native N-glycosites in the D1 domain (N65, N79) were maintained, while the native glycosites in the D2 domain were mutated to glutamine (N141Q, N177Q, N186Q, N198Q). Third, the glycoengineered polypeptide was modified by adding a peptide sequence containing an N-glycosylation consensus site (‘glycotag’; SEQ ID NO: 37) and a His tag (FLGT13; SEQ ID NO: 40) for purification to the C-terminus. These construction steps resulted in the sCD89 glycoengineered polypeptide having three N-glycosylation sites (two native sites and a third engineered site within a C-terminal glycotag sequence; SEQ ID NO: 41).

The corresponding nucleotide sequence was transfected as an expression cassette into glycoengineered Leishmania tarentolae host cell line StCGP4564. The resulting cell line StCGP5907 was grown in a bioreactor for sCD89 expression and secretion. sCD89 containing A2GalNAc2 glycans was then purified from the cell supernatant via an IMAC purification and analyzed (Table 2). Size exclusion chromatography (SEC) was used to measure aggregation and degradation of the protein.

Results: The glycoengineering process resulted in the sCD89 glycoengineered polypeptide. Analytical data of the construct (Table 2) demonstrated that the sCD89 glycoengineered polypeptide has a high percentage of A2GalNAc2 (82.8%) and high occupancy of all three glycosites (>95% with all occupied).

TABLE 2
Analytical data for an exemplary sCD89 glycoengineered polypeptide

| | | | SEC | | | | Glycan |
|---|---|---|---|---|---|---|---|
| | | Endotoxin | | Degradation/ | | QC | Occupancy |
| Protein Name | N-glycan | (LAL) | Monomer | LMW | Aggregation | N-glycan | (IP-MS) |
| sCD89 | A2GalNAc2 | <1 | 47% | 53% | 0% | 82.8% | 3 |
| glycoengineered | | EU/mg | | | | A2GalNAc2 | glycosites >95% |
| polypeptide | | | | | | | all occupied |

### Example 2: Glycoengineered sCD89 Polypeptides Deplete IgA Antibodies

This example demonstrates depletion of human IgA antibodies in a rat model with an exemplary glycoengineered polypeptide comprising an sCD89 polypeptide.

Methods: Experiments employed standard animal clinical monitoring. Eight female Wistar rats were first injected intravenously with 1 mg of purified total human IgA on Day 1 (D1) at −0.5 hours (30 minutes prior to dosing with glycoengineered polypeptide, see below) to achieve theoretical circulating levels of IgA close to 0.1 mg/mL immediately after injection (theoretical Czero). For reference, it is noted that typically, in humans, the level of circulating IgA is about 2.6 mg/mL. Next, at 0 hours (30 minutes after administration of IgA), rats were injected subcutaneously with 2.0 mg of an exemplary sCD89 glycoengineered polypeptide (four animals) or a control PBS solution (four animals). Blood samples were acquired at 1, 3, 6, 10, 24, and 48 hours after the injection of the exemplary glycoengineered polypeptide or PBS control. All blood samples were processed to serum and distributed into 4 vials which were stored at −80° C. until analysis. Human IgA levels were evaluated in serum using a human IgA quantification ELISA kit following the manufacturer's instructions (Catalog #E88-102, Bethyl laboratories). Since binding of IgA by glycoengineered sCD89 polypeptide did not interfere with detection of human IgA by the ELISA kit (data not shown, verified during assay development), the ELISA method therefore quantifies total (free+bound) human IgA. Results are expressed as a percent of target (IgA) remaining in the serum sample.

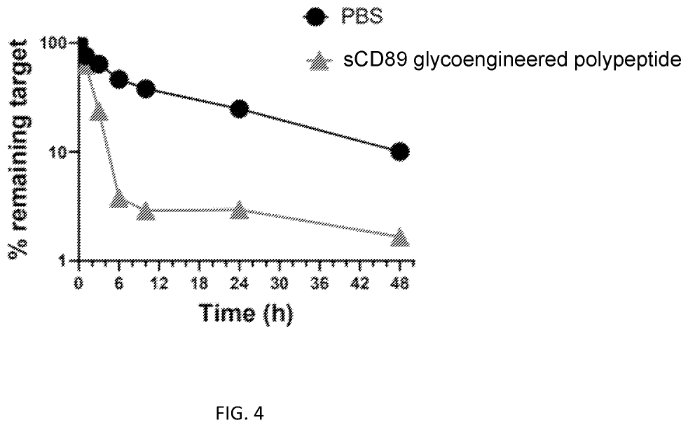

Results: The percentage of human IgA target remaining in rat serum was rapidly depleted following administration of the exemplary glycoengineered polypeptide. At 6 hours following administration, less than 5% of theoretical Czero IgA remained in the serum of samples treated with the sCD89 glycoengineered polypeptide, while most of the IgA remained in the serum from PBS treated samples (FIG. 4). The maximal effect of IgA depletion in the group treated with the sCD89 glycoengineered polypeptide, as compared to PBS control group, was achieved at 6 hours.

This data demonstrates in vivo depletion of human IgA in rats with subcutaneous administration of the sCD89 glycoengineered polypeptide. This data supports the development of glycoengineered polypeptides for use as therapeutic agent in treating and/or preventing IgAN (e.g., in a subject having one or more IgA antibodies). This data further supports the use of glycoengineered polypeptides for reducing the level of IgA antibodies in a subject.

### Example 3: Identifying Glycoengineered Polypeptides Binding to IgA

This example describes a process that can be used to identify polypeptides that can preferentially bind to IgA antibodies which have a particular glycosylation profile. For example, the particular glycosylation profile can be a glycan profile that is rare on wild-type IgA and increased on galactose-deficient IgA1. In some embodiments, a glycan profile on galactose-deficient IgA1 is characterized as having a terminal GalNac. Such a glycan profile is also known in the field as a “Tn antigen.” See, e.g., Knoppova et al., Frontiers in Immunology (2016) volume 7, article 117, the entire contents of which are hereby incorporated by reference.

Methods: To identify exemplary polypeptides which can bind to IgA1 having a particular glycosylation profile (e.g., Tn-IgA), first humanized or hyperimmune mice strains are immunized with peptides mimicking IgA1 hinge Tn O-glycosylation. This immunization generates a diverse library of fully human binders that can recognize and bind to the antigen (Tn-IgA). Once a library of binders is obtained, specificity of the binders is determined by comparing binding to Tn-IgA1 versus wild-type IgA1 (in which Tn O-glycosylation is rare). Binding affinity is quantified using standard methods such as Biacore assays. Tn-IgA1 is obtained by treating wt-IgA1 with neuraminidase (Sigma ref #10269611001) and β-galactosidase (sigma ref #G4142-0.2UN) enzymes, according to standard protocol. This treatment converts the normal O-glycosylation profile of wild-type-IgA1 into a profile enriched for the Tn antigen.

Results: Polypeptides with a binding specificity preferential for galactose deficient IgA1 (Tn-IgA1) as compared to wt-IgA1 are identified. In some embodiments, polypeptides that bind preferentially to a preferred glycosylation profile on IgA1 (e.g., Tn-IgA) demonstrate increased binding to Tn-IgA1 compared to normal IgA1.

Polypeptides identified using this method can be used to construct glycoengineered polypeptides that can bind specifically to galactose deficient IgA1, e.g., using the methods described in Example 1 herein. In some embodiments, glycoengineered polypeptides comprising one or more polypeptides identified using the method disclosed in this Example demonstrate preferential binding to IgAN patient antibodies and can be useful as a therapeutic agent in treating and/or preventing IgAN (e.g., in a subject having one or more IgA autoantibodies).

### ENUMERATED EMBODIMENTS

Embodiment 1. A glycoengineered polypeptide comprising:
(a) a first moiety comprising one or more peptides that specifically binds to a target antibody or a fragment or a complex thereof; and
(b) a second moiety comprising one or more glycans conjugated to the first moiety at one or more glycosylation sites.

Embodiment 2. The glycoengineered polypeptide of embodiment 1, wherein a complex comprising a target antibody is or comprises an immune complex.

Embodiment 3. The glycoengineered polypeptide of embodiment 1 or 2, wherein the target antibody comprises galactose-deficient IgA1 (gd-IgA1), or a fragment or a complex thereof.

Embodiment 4. The glycoengineered polypeptide of embodiment 1 or 2, wherein the target antibody comprises IgA1, or a fragment or a complex thereof.

Embodiment 5. The glycoengineered polypeptide of embodiment 1 or 2, wherein the target antibody comprises an autoantibody that specifically binds to gd-IgA1 (“anti-gd-IgA1 autoantibody”), or a fragment or a complex thereof.

Embodiment 6. The glycoengineered polypeptide of any one of the preceding embodiments, wherein the second moiety specifically binds to one or more endocytic receptors.

Embodiment 7. The glycoengineered polypeptide of embodiment 6, wherein the endocytic receptor is or comprises an endocytic lectin.

Embodiment 8. The glycoengineered polypeptide of embodiment 6 or 7, wherein the endocytic receptor is chosen from: an asialoglycoprotein receptor (ASGPR); a mannose binding receptor, a Cluster of Differentiation 206 (CD206) receptor; a DC-SIGN (Cluster of Differentiation 209 or CD209) receptor; a C-Type Lectin Domain Family 4 Member G (LSECTin) receptor; a macrophage inducible Ca2+-dependent lectin receptor (Mincle); a L-SIGN CD209L receptor; dectin-1; dectin-2, langerin, macrophage mannose 2 receptor, BDCA-2, DCIR, MBL, MDL, MICL, CLEC2, CLEC10, DNGR1, CLEC12B, DEC-205, and mannose 6 phosphate receptor (M6PR), or a combination thereof.

Embodiment 9. The glycoengineered polypeptide of any one of the preceding embodiments, wherein the glycan comprises a terminal GlcNac.

Embodiment 10. The glycoengineered polypeptide of any one of embodiments 1-8, wherein the glycan comprises a terminal GalNac.

Embodiment 11. The glycoengineered polypeptide of any one of embodiments 1-8, wherein the glycan comprises a terminal Gal.

Embodiment 12. The glycoengineered polypeptide of any one of the preceding embodiments, wherein the glycan is an N-glycan.

Embodiment 13. The glycoengineered polypeptide of embodiment 12, wherein the N-glycan is linked to the glycoengineered polypeptide at 1, 2, 3, 4 or 5 N-glycosylation sites.

Embodiment 14. The glycoengineered polypeptide of any one of the preceding embodiments, wherein the glycan structure comprises GlcNAc2-Man3-GlcNAc2, GalNAc2-GlcNAc2-Man3-GlcNAc2, Gal2-GlcNAc2-Man3-GlcNAc2, GlcNAc1-Man3-GlcNAc2, Gal2-GlcNAc2-Man3-GlcNAc2, Gal1-GlcNAc2-Man3-GlcNAc2, GalNAc1-GlcNAc2-Man3-GlcNAc2, GlcNAc3-Man3-GlcNAc2, GlcNAc4-Man3-GlcNAc2, Gal3-GlcNAc3-Man3-GlcNAc2, GalNAc3-GlcNAc3-Man3-GlcNAc2, GalNAc4-GlcNAc4-Man3-GlcNAc2, Gal4-GlcNAc4-Man3-GlcNAc2, or Man-6-P-N-glycan.

Embodiment 15. The glycoengineered polypeptide of any one of the preceding embodiments, wherein increasing a number of glycan structures on the glycoengineered polypeptide increases the rate of lysosomal degradation as compared to an otherwise similar glycoengineered polypeptide with fewer glycan structures.

Embodiment 16. The glycoengineered polypeptide of any one of the preceding embodiments, wherein a number of glycan structures comprise 1 or more, 2 or more, 3 or more, 4 or more, 5 or more, 6 or more, 7 or more, 8 or more, 9 or more or 10 or more.

Embodiment 17. The glycoengineered polypeptide of any one of the preceding embodiments, wherein the glycan structure comprises a monoantennary structure, biantennary structure, a triantennary structure, or a tetraantennary structure.

Embodiment 18. The glycoengineered polypeptide of any one of the preceding embodiments, wherein the glycan structure comprises a biantennary structure,

Embodiment 19. The glycoengineered polypeptide of embodiment 18, wherein the glycan structure comprises a biantennary GalNAc.

Embodiment 20. The glycoengineered polypeptide of embodiment 18 or 19, wherein the biantennary GalNac binds to an asialoglycoprotein receptor (ASGPR) or a fragment or variant thereof, or a complex comprising ASGPR.

Embodiment 21. The glycoengineered polypeptide of any one of embodiments 12-20, wherein the N-glycan has a structure of:

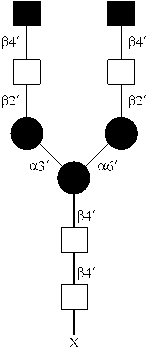

wherein the black square represents an N-acetyl galactosamine (GalNAc), the white square represents an N-acetylglucosamine (GlcNAc) residue and the black circle represents a mannose (Man) residue, and wherein X represents an amino acid residue of the first moiety.

Embodiment 22. The glycoengineered polypeptide of any one of embodiments 12-21, wherein the N-glycan is conjugated to the glycoengineered polypeptide at at least one, two, three, or four N-glycosylation sites.

Embodiment 23. The glycoengineered polypeptide of any one of embodiments 12-21, wherein the N-glycan is conjugated to the glycoengineered polypeptide at one, two, three, or four N-glycosylation sites.

Embodiment 24. The glycoengineered polypeptide of any one of the preceding embodiments, wherein the N-glycosylation site comprises a consensus sequence of N-X-S/T or N-X-C, wherein X is any amino acid except proline.

Embodiment 25. The glycoengineered polypeptide of any one of the preceding embodiments, wherein the N-glycosylation site is naturally occurring.

Embodiment 26. The glycoengineered polypeptide of embodiment 25, wherein the N-glycosylation site is engineered into the amino acid sequence of the first moiety, optionally wherein the engineered N-glycosylation site comprises the sequence of SEQ ID NO: 37.

Embodiment 27. The glycoengineered polypeptide of any one of embodiments 6-26, wherein the endocytic receptor is or comprises ASGPR or a fragment or variant thereof, or a complex comprising ASGPR.

Embodiment 28. The glycoengineered polypeptide of embodiment 27, wherein when the endocytic receptor is ASGPR, the glycan structure of the second moiety comprises a terminal GalNac.

Embodiment 29. The glycoengineered polypeptide of any one of the preceding embodiments, wherein the first moiety comprises one or more peptides that specifically bind to a glycosylation-deficient IgA1 (gd-IgA1), or a fragment or a complex thereof.

Embodiment 30. The glycoengineered polypeptide of embodiment 29, wherein the gd-IgA1 comprises a hinge region that is glycosylated.

Embodiment 31. The glycoengineered polypeptide of embodiment 30, wherein the hinge region comprises the sequence of PVPSTPPTPSPSTPPTPSPSC (SEQ ID NO: 1) or a variant or a fragment thereof.

Embodiment 32. The glycoengineered polypeptide of embodiment 30 or 31, wherein the hinge region comprises nine O-glycosylation sites.

Embodiment 33. The glycoengineered polypeptide of embodiment 32, wherein at least 1, 2, 3, 4, 5, or 6 of the O-glycosylation sites are occupied.

Embodiment 34. The glycoengineered polypeptide of any one of embodiments 29-33, wherein the one or more peptides that specifically bind to gd-IgA1 recognize a glycan profile on gd-IgA1.

Embodiment 35. The glycoengineered polypeptide of embodiment 34, wherein the gd-IgA1 comprises a glycan profile that is different from a reference glycan profile of an IgA1.

Embodiment 36. The glycoengineered polypeptide of embodiment 34 or 35, wherein the gd-IgA1 comprises a glycan profile having at least one less glycan compared to a reference glycan profile of an IgA1.

Embodiment 37. The glycoengineered polypeptide of any one of embodiments 34-36, wherein the reference glycan profile comprises an O-glycosylation profile.

Embodiment 38. The glycoengineered polypeptide of embodiment 37, wherein the reference O-glycosylation profile comprises 1, 2, 3, 4, 5, or 6 O-glycan chains.

Embodiment 39. The glycoengineered polypeptide of embodiment 37 or 38, wherein the O-glycan chains of the reference O-glycosylation profile comprises N-acetylgalactosamine (GalNac).

Embodiment 40. The glycoengineered polypeptide of any one of embodiments 37-39, wherein the O-glycan chain further comprises a Galactose (Gal), a sialic acid or a combination thereof.

Embodiment 41. The glycoengineered polypeptide of any one of embodiments 34-40, wherein the gd-IgA1 glycan profile comprises at least one less galactosylation compared to the reference glycan profile.

Embodiment 42. The glycoengineered polypeptide of any one of embodiments 34-41, wherein the gd-IgA1 glycan profile comprises at least 5% less galactosylation compared to the reference glycan profile.

Embodiment 43. The glycoengineered polypeptide of any one of embodiments 34-42, wherein the gd-IgA1 glycan profile comprises increased sialylation compared to the reference glycan profile.

Embodiment 44. The glycoengineered polypeptide of any one of embodiments 34-43, wherein the gd-IgA1 glycan profile comprises a terminal GalNac (also referred to as the Tn antigen).

Embodiment 45. The glycoengineered polypeptide of any one of embodiments 34-43, wherein the gd-IgA1 glycan profile comprises GalNac with an alpha2,6 linked sialic acid (also referred to as the STn antigen).

Embodiment 46. The glycoengineered polypeptide of any one of embodiments 34-45, wherein sialylation of a terminal GalNac blocks effective galactosylation.

Embodiment 47. The glycoengineered polypeptide of any one of embodiments 29-46, wherein the one or more peptides that specifically bind to gd-IgA1 recognize an epitope in a hinge region of gd-IgA1.

Embodiment 48. The glycoengineered polypeptide of embodiment 47, wherein the altered glycan profile results in a conformational change in gd-IgA1.

Embodiment 49. The glycoengineered polypeptide of embodiment 48, wherein the conformation change exposes a neoepitope that is recognized by the one or more peptides.

Embodiment 50. The glycoengineered polypeptide of embodiment 49, wherein the neoepitope is not present in non-galactose deficient IgA1, e.g., IgA1 comprising a reference glycan profile.

Embodiment 51. The glycoengineered polypeptide of embodiment 49 or 50, wherein the neoepitope is a linear epitope.

Embodiment 52. The glycoengineered polypeptide of embodiment 49 or 50, wherein the neoepitope is a conformational epitope.

Embodiment 53. The glycoengineered polypeptide of any one of embodiments 29-52, wherein the first moiety comprises 1, 2, 3, 4, 5, or more peptides that specifically bind to gd-IgA1 or a fragment thereof.

Embodiment 54. The glycoengineered polypeptide of any one of embodiments 29-53, wherein the one or more peptides that specifically bind to gd-IgA1 or a fragment thereof are the same.

Embodiment 55. The glycoengineered polypeptide of embodiment 54, wherein the one or more peptides that specifically bind to gd-IgA1 or a fragment thereof are separated by an intervening sequence.

Embodiment 56. The glycoengineered polypeptide of embodiment 55, wherein the intervening sequence is an IRES, a protease cleavage site, a linker or a spacer or a combination thereof.

Embodiment 57. The glycoengineered polypeptide of any one of embodiments 29-53, wherein the one or more peptides that specifically bind to gd-IgA1 or a fragment thereof are different.

Embodiment 58. The glycoengineered polypeptide of embodiment 57, wherein the one or more peptides that specifically bind to gd-IgA1 or a fragment thereof are separated by an intervening sequence.

Embodiment 59. The glycoengineered polypeptide of embodiment 58, wherein the intervening sequence is an IRES, a protease cleavage site, a linker or a spacer or a combination thereof.

Embodiment 60. The glycoengineered polypeptide of any one of embodiments 57-59, wherein the different peptides form a spatial epitope.

Embodiment 61. The glycoengineered polypeptide of any one of embodiments 29-60, wherein the one or more peptides that specifically bind to gd-IgA1 or a fragment thereof are each conjugated to a second moiety.

Embodiment 62. The glycoengineered polypeptide of any one of embodiments 29-60, wherein the one or more peptides that specifically bind to gd-IgA1 or a fragment thereof are not each conjugated to the second moiety.

Embodiment 63. The glycoengineered polypeptide of any one of embodiments 29-62, wherein the one or more peptides that specifically bind to gd-IgA1 or a fragment thereof are conjugated to each other.

Embodiment 64. The glycoengineered polypeptide of embodiment 63, wherein the one or more peptides are situated on one polypeptide.

Embodiment 65. The glycoengineered polypeptide of embodiment 63 or 64, wherein the one or more peptides are separated by an intervening amino acid sequence.

Embodiment 66. The glycoengineered polypeptide of embodiment 65, wherein the intervening amino acid sequence is an IRES, a protease cleavage site, a linker or a spacer or a combination thereof.

Embodiment 67. The glycoengineered polypeptide of any one of embodiments 29-66, wherein the one or more peptides that specifically bind to gd-IgA1 comprises an antibody agent.

Embodiment 68. The glycoengineered polypeptide of embodiment 67, wherein the antibody agent comprises an antigen binding fragment.

Embodiment 69. The glycoengineered polypeptide of embodiment 68, wherein the antibody agent comprises a full antibody, a Fab fragment, an scFv, a nanobody, a duobody, a single domain antibody (e.g., a VHH).

Embodiment 70. The glycoengineered polypeptide of embodiment 68, wherein the antibody agent comprises a VHH, e.g., a camelid VHH or a bivalent VHH.

Embodiment 71. The glycoengineered polypeptide of any one of embodiments 1-28, wherein the first moiety comprises one or more peptides that specifically bind to IgA1, or a fragment or a complex thereof.

Embodiment 72. The glycoengineered polypeptide of embodiment 71, wherein the first moiety comprises 1, 2, 3, 4, 5, or more peptides that specifically bind to IgA1 or a fragment thereof.

Embodiment 73. The glycoengineered polypeptide of embodiment 71 or 72, wherein the one or more peptides that specifically bind to IgA1 or a fragment thereof are the same.

Embodiment 74. The glycoengineered polypeptide of embodiment 73, wherein the one or more peptides that specifically bind to IgA1 or a fragment thereof are separated by an intervening sequence.

Embodiment 75. The glycoengineered polypeptide of embodiment 74, wherein the intervening sequence is an IRES, a protease cleavage site, a linker or a spacer or a combination thereof.

Embodiment 76. The glycoengineered polypeptide of embodiment 71 or 72, wherein the one or more peptides that specifically bind to IgA1 or a fragment thereof are different.

Embodiment 77. The glycoengineered polypeptide of embodiment 76, wherein the one or more peptides that specifically bind to IgA1 or a fragment thereof are separated by an intervening sequence.

Embodiment 78. The glycoengineered polypeptide of embodiment 77, wherein the intervening sequence is an IRES, a protease cleavage site, a linker or a spacer or a combination thereof.

Embodiment 79. The glycoengineered polypeptide of any one of embodiments 76-78, wherein the different peptides form a spatial epitope.

Embodiment 80. The glycoengineered polypeptide of any one of embodiments 71-79, wherein the one or more peptides that specifically bind to IgA1 or a fragment thereof are each conjugated to a second moiety.

Embodiment 81. The glycoengineered polypeptide of any one of embodiments 71-79, wherein the one or more peptides that specifically bind to IgA1 or a fragment thereof are not each conjugated to the second moiety.

Embodiment 82. The glycoengineered polypeptide of any one of embodiments 71-81, wherein the one or more peptides that specifically bind to IgA1 or a fragment thereof are conjugated to each other.

Embodiment 83. The glycoengineered polypeptide of embodiment 82, wherein the one or more peptides are situated on one polypeptide.

Embodiment 84. The glycoengineered polypeptide of embodiment 82 or 83, wherein the one or more peptides are separated by an intervening amino acid sequence.

Embodiment 85. The glycoengineered polypeptide of embodiment 84, wherein the intervening amino acid sequence is an IRES, a protease cleavage site, a linker or a spacer or a combination thereof.

Embodiment 86. The glycoengineered polypeptide of any one of embodiments 71-85, wherein the glycoengineered polypeptide comprises a first moiety comprising one peptides that specifically binds to IgA1 or a fragment thereof or a fragment thereof.

Embodiment 87. The glycoengineered polypeptide of any one of embodiments 71-86, wherein an IgA1 amino acid sequence is provided as SEQ ID NO: 2.

Embodiment 88. The glycoengineered polypeptide of any one of embodiments 71-87, wherein the one or more peptides that specifically bind to IgA1 recognize an epitope on IgA1.

Embodiment 89. The glycoengineered polypeptide of embodiment 88, wherein the epitope is a conformational epitope.

Embodiment 90. The glycoengineered polypeptide of embodiment 88, wherein the epitope is a linear epitope.

Embodiment 91. The glycoengineered polypeptide of any one of embodiments 71-90, wherein the first moiety comprising one or more peptides that specifically bind to IgA1, or a fragment or a complex thereof comprises a CD89 (FcaR1) polypeptide, or a variant, or a fragment thereof.

Embodiment 92. The glycoengineered polypeptide of embodiment 91, wherein the CD89 comprises soluble CD89 or a fragment or variant thereof.

Embodiment 93. The glycoengineered polypeptide of embodiment 91 or 92, wherein a CD89 polypeptide is provided as SEQ ID NO: 5 with or without a signal peptide, SEQ ID NO: 9 with or without a signal peptide, SEQ ID NO: 38 with or without a signal peptide or SEQ ID NO: 39.

Embodiment 94. The glycoengineered polypeptide of any one of embodiments 91-93, wherein the one or more peptides that specifically bind to IgA1, or a fragment or a complex thereof comprises a fragment of a CD89 polypeptide.

Embodiment 95. The glycoengineered polypeptide of embodiment 94, wherein the fragment comprises at least 5% of a full length CD89 polypeptide with or without a signal peptide.

Embodiment 96. The glycoengineered polypeptide of embodiment 94, wherein the fragment comprises no more than 99% of a full length CD89 polypeptide with or without a signal peptide.

Embodiment 97. The glycoengineered polypeptide of any one of embodiments 91-96, wherein the fragment comprises a CD89 amino acid sequence that binds to IgA1.

Embodiment 98. The glycoengineered polypeptide of any one of embodiments 91-97, wherein the fragment comprises one or more additional amino acid sequences 5′ and/or 3′ to the fragment sequence.

Embodiment 99. The glycoengineered polypeptide of any one of embodiments 91-98, wherein the one or more peptides that specifically bind to IgA1, or a fragment or a complex thereof comprises a variant of a CD89 polypeptide.

Embodiment 100. The glycoengineered polypeptide of any one of embodiments 91-99, wherein the one or more peptides that specifically bind to an IgA1 antibody comprises a contiguous chain of amino acids comprising at least 5% of the amino acid of SEQ ID NO: 5, SEQ ID NO: 9, SEQ ID NO: 38 or SEQ ID NO: 39.

Embodiment 100.1. The glycoengineered polypeptide of any one of embodiments 91-100, wherein the one or more peptides that specifically bind to IgA1, or a fragment or a complex thereof comprises a sequence having at least 85% identity to
(i) SEQ ID NO: 5, or
(ii) SEQ ID NO: 5, without the signal peptide, optionally wherein the sequence further comprises a different signal peptide, e.g., as disclosed herein.

Embodiment 100.2. The glycoengineered polypeptide of any one of embodiments 91-100, wherein the one or more peptides that specifically bind to IgA1, or a fragment or a complex thereof comprises a sequence having at least 85% identity to:
(i) SEQ ID NO: 9, or
(ii) SEQ ID NO: 9, without the signal peptide, optionally wherein the sequence further comprises a different signal peptide, e.g., as disclosed herein.

Embodiment 100.3. The glycoengineered polypeptide of any one of embodiments 91-100, wherein the one or more peptides that specifically bind to IgA1, or a fragment or a complex thereof comprises a sequence having at least 85% identity to:
(i) SEQ ID NO: 38, or
(ii) SEQ ID NO: 38, without the signal peptide, optionally wherein the sequence further comprises a different signal peptide, e.g., as disclosed herein.

Embodiment 100.4. The glycoengineered polypeptide of any one of embodiments 91-100, wherein the one or more peptides that specifically bind to IgA1, or a fragment or a complex thereof, comprise a sequence having SEQ ID NO: 39, optionally wherein the sequence further comprises a signal peptide, e.g., as disclosed herein. Embodiment 100.5. The glycoengineered polypeptide of any one of embodiments 91-100, wherein the one or more peptides that specifically bind to IgA1 comprises a sequence having at least 85% identity to SEQ ID NO: 41.

Embodiment 101. The glycoengineered polypeptide of any one of embodiments 71-100.2, wherein the one or more peptides that specifically bind to IgA1 comprises an antibody agent.

Embodiment 102. The glycoengineered polypeptide of embodiment 101, wherein the antibody agent comprises an antigen binding fragment.

Embodiment 103. The glycoengineered polypeptide of embodiment 102, wherein the antibody agent comprises a full antibody, a Fab fragment, an scFv, a nanobody, a duobody, a single domain antibody (e.g., a VHH).

Embodiment 104. The glycoengineered polypeptide of embodiment 102, wherein the antibody agent comprises a VHH, e.g., a camelid VHH or a bivalent VHH.

Embodiment 105. The glycoengineered polypeptide of any one of embodiments 1-27, wherein the first moiety comprises one or more peptides that specifically bind to an anti-gd-IgA1 autoantibody.

Embodiment 106. The glycoengineered polypeptide of embodiment 105, wherein the first moiety comprises 1, 2, 3, 4, 5, or more peptides that specifically bind to Anti-gd-IgA1 or a fragment thereof.

Embodiment 107. The glycoengineered polypeptide of embodiment 105 or 106, wherein the one or more peptides that specifically bind to Anti-gd-IgA1 or a fragment thereof are the same.

Embodiment 108. The glycoengineered polypeptide of embodiment 107, wherein the one or more peptides that specifically bind to Anti-gd-IgA1 or a fragment thereof are separated by an intervening sequence.

Embodiment 109. The glycoengineered polypeptide of embodiment 107, wherein the intervening sequence is an IRES, a protease cleavage site, a linker or a spacer or a combination thereof.

Embodiment 110. The glycoengineered polypeptide of embodiment 108 or 109, wherein the one or more peptides that specifically bind to Anti-gd-IgA1 or a fragment thereof are different.

Embodiment 111. The glycoengineered polypeptide of embodiment 110, wherein the one or more peptides that specifically bind to Anti-gd-IgA1 or a fragment thereof are separated by an intervening sequence.

Embodiment 112. The glycoengineered polypeptide of embodiment 111, wherein the intervening sequence is an IRES, a protease cleavage site, a linker or a spacer or a combination thereof.

Embodiment 113. The glycoengineered polypeptide of any one of embodiments 105-112, wherein the different peptides form a spatial epitope.

Embodiment 114. The glycoengineered polypeptide of any one of embodiments 105-112, wherein the one or more peptides that specifically bind to Anti-gd-IgA1 or a fragment thereof are each conjugated to a second moiety.

Embodiment 115. The glycoengineered polypeptide of any one of embodiments 105-114, wherein the one or more peptides that specifically bind to Anti-gd-IgA1 or a fragment thereof are not each conjugated to the second moiety.

Embodiment 116. The glycoengineered polypeptide of any one of embodiments 105-115, wherein the one or more peptides that specifically bind to Anti-gd-IgA1 or a fragment thereof are conjugated to each other.

Embodiment 117. The glycoengineered polypeptide of embodiment 116, wherein the one or more peptides are situated on one polypeptide.

Embodiment 118. The glycoengineered polypeptide of embodiment 116 or 117, wherein the one or more peptides are separated by an intervening amino acid sequence.

Embodiment 119. The glycoengineered polypeptide of embodiment 118, wherein the intervening amino acid sequence is an IRES, a protease cleavage site, a linker or a spacer or a combination thereof.

Embodiment 120. The glycoengineered polypeptide of any one of embodiments 105-119, wherein the glycoengineered polypeptide comprises a first moiety comprising one peptides that specifically binds to Anti-gd-IgA1 or a fragment thereof or a fragment thereof.

Embodiment 121. The glycoengineered polypeptide of any one of embodiments 105-120, wherein the anti-gd-IgA1 autoantibody is an IgG, an IgM, an IgE, an IgD, or an IgM.

Embodiment 122. The glycoengineered polypeptide of any one of embodiments 105-121, wherein the anti-gd-IgA1 autoantibody is an IgG.

Embodiment 123. The glycoengineered polypeptide of embodiment 122, wherein the IgG comprises a mutation in a complementarity determining region 3 (CDR3) of an Ig heavy chain (IgH) variable region.

Embodiment 124. The glycoengineered polypeptide of embodiment 123, wherein the mutation comprises an Alanine to Serine mutation.

Embodiment 125. The glycoengineered polypeptide of embodiment 124, wherein the Alanine to Serine mutation occurs in a YCAR amino acid sequence or a YCAK amino acid sequence of an IgH.

Embodiment 126. The glycoengineered polypeptide of any one of embodiments 105-125, wherein the first moiety comprises one or more peptides that specifically bind to mutation in a CDR3 IgH region an anti-gd-IgA1 autoantibody.

Embodiment 127. The glycoengineered polypeptide of any one of embodiments 105-126, wherein the first moiety comprises one or more peptides that specifically bind to a YCAR amino acid sequence in a CDR3 IgH in which the Alanine is mutated to a Serine.

Embodiment 128. The glycoengineered polypeptide of any one of embodiments 105-126, wherein the first moiety comprises one or more peptides that specifically bind to a YCAK amino acid sequence in a CDR3 IgH in which the Alanine is mutated to a Serine.

Embodiment 129. The glycoengineered polypeptide of any one of embodiments 105-128, wherein the one or more peptides that specifically bind to an anti-gd-IgA1 autoantibody binds to an IgG protein, or a fragment or a variant thereof.

Embodiment 130. The glycoengineered polypeptide of embodiment 129, wherein the IgG is an IgG1, an IgG2, an IgG3, or an IgG4.

Embodiment 131. The glycoengineered polypeptide of embodiment 129 or 130, wherein a wildtype IgG1 protein is provided as SEQ ID NO: 3.

Embodiment 132. The glycoengineered polypeptide of embodiment 129 or 130, wherein a wildtype IgG4 protein is provided as SEQ ID NO: 4.

Embodiment 133. The glycoengineered polypeptide of any one of embodiments 105-132, wherein the one or more peptides that specifically bind to an anti-gd-IgA1 autoantibody binds to a variant of an IgG protein.

Embodiment 134. The glycoengineered polypeptide of any one of embodiments 105-132, wherein the first moiety comprising one or more peptides that specifically bind to an anti-gd-IgA1 autoantibody comprises a gd-IgA1 polypeptide, or a variant, or fragment thereof.

Embodiment 135. The glycoengineered polypeptide of embodiment 134, wherein the gd-IgA1 comprises a hinge region that is glycosylated.

Embodiment 136. The glycoengineered polypeptide of embodiment 135, wherein the hinge region comprises the sequence of PVPSTPPTPSPSTPPTPSPSC (SEQ ID NO: 1) or a fragment thereof.

Embodiment 137. The glycoengineered polypeptide of embodiment 135 or 136, wherein the hinge region comprises nine O-glycosylation sites.

Embodiment 138. The glycoengineered polypeptide of embodiment 137, wherein at least 1, 2, 3, 4, 5, or 6 of the O-glycosylation sites are occupied.

Embodiment 139. The glycoengineered polypeptide of any one of embodiments 134-138, wherein the first moiety comprises one or more peptides that specifically bind to an anti-gd-IgA1 autoantibody comprises a gd-IgA1 polypeptide fragment.

Embodiment 140. The glycoengineered polypeptide of embodiment 139, wherein the fragment comprises the sequence of SEQ ID NO: 1, or a fragment or a variant thereof.

Embodiment 141. The glycoengineered polypeptide of embodiment 140, wherein the fragment comprises one or more additional amino acid residues to the 5′ and/or 3′ end of the sequence.

Embodiment 142. The glycoengineered polypeptide of any one of embodiments 134-142, wherein the fragment has a glycan profile that is different from a reference glycan profile.

Embodiment 143. The glycoengineered polypeptide of embodiment 142, wherein the fragment has a glycan profile having a terminal GalNac.

Embodiment 144. The glycoengineered polypeptide of embodiment 142, wherein the fragment has a glycan profile having a terminal sialylated GalNac.

Embodiment 145. The glycoengineered polypeptide of any one of embodiments 134-144, wherein the fragment comprises a gd-IgA1 sequence that leads to capping of GlcNac or GalNac with sialic acid.

Embodiment 146. The glycoengineered polypeptide of any one of embodiments 105-145, wherein the one or more peptides that specifically bind to anti-gd-IgA1 comprises an antibody agent.

Embodiment 147. The glycoengineered polypeptide of embodiment 146, wherein the antibody agent comprises an antigen binding fragment.

Embodiment 148. The glycoengineered polypeptide of embodiment 147, wherein the antibody agent comprises a full antibody, a Fab fragment, an scFv, a nanobody, a duobody, a single domain antibody (e.g., a VHH).

Embodiment 149. The glycoengineered polypeptide of embodiment 147, wherein the antibody agent comprises a VHH, e.g., a camelid VHH or a bivalent VHH.

Embodiment 150. The glycoengineered polypeptide of any one of the preceding embodiments, wherein the first moiety comprises:
(i) one or more peptides that specifically bind to gd-IgA1 or a fragment or complex thereof;
(ii) one or more peptides that specifically bind to IgA1 or a fragment or complex thereof;
(iii) one or more peptides that specifically bind to an anti-gd-IgA1 autoantibody or a fragment or complex thereof;
(iv) any one or all of (i)-(iii).

Embodiment 151. The glycoengineered polypeptide of embodiment 150, wherein (i), (ii) and/or (iii) are situated on the same polypeptide.

Embodiment 152. The glycoengineered polypeptide of embodiment 150, wherein (i), (ii) and/or (iii) are situated on different polypeptides.

Embodiment 153. The glycoengineered polypeptide of embodiment 152, wherein (i) is situated on a first glycoengineered polypeptide, (ii) is situated on a second glycoengineered polypeptide, and/or (iii) is situated on a third glycoengineered polypeptide.

Embodiment 154. The glycoengineered polypeptide of embodiment 150, wherein each of (i), (ii) and (iii) are conjugated to a second moiety.

Embodiment 155. The glycoengineered polypeptide of any one of the preceding embodiments, wherein the target antibody bound by the first moiety comprises an immune complex, optionally wherein the immune complex comprises:
the target antibody or a fragment thereof, an antigen recognized by the target antibody, one or more components of a complement system, one or more additional immunoglobulins, or combinations thereof.

Embodiment 156. The glycoengineered polypeptide of embodiment 155, wherein:
(i) the one or more immunoglobulins comprises an IgG, an IgA, an IgM, an IgD, an IgE, or fragments or combinations thereof; and/or
(ii) the immune complex comprises a gd-IgA1 antibody, an IgA1 antibody, an anti-gd-IgA1 autoantibody, or combinations thereof.

Embodiment 157. The glycoengineered polypeptide of embodiment 156, wherein the anti-gd-IgA1 autoantibody is an IgG or an IgM.

Embodiment 158. The glycoengineered polypeptide of any one of the preceding embodiments, wherein the polypeptide comprises one or more additional elements.

Embodiment 159. The glycoengineered polypeptide of embodiment 158, wherein the additional element comprises a linker, a signal peptide (e.g., SEQ ID NO: 21, SEQ ID NO: 22, SEQ ID NO: 42, SEQ ID NO: 43, SEQ ID NO: 44, or a portion of any of the foregoing), a tag, a half-life extender or combinations thereof.

Embodiment 160. The glycoengineered polypeptide of any one of the preceding embodiments, wherein the second moiety is conjugated to the first moiety at at least one, two, three, or four N-glycosylation sites.

Embodiment 161. The glycoengineered polypeptide of any one of the preceding embodiments, wherein the second moiety is conjugated to the first moiety at one, two, three, or four N-glycosylation sites.

Embodiment 162. The glycoengineered polypeptide of any one of the preceding embodiments, wherein the N-glycosylation site comprises a consensus sequence of N-X-S/T or N-X-C, wherein X is any amino acid except proline.

Embodiment 163. The glycoengineered polypeptide of any one of the preceding embodiments, wherein the second moiety is conjugated to the first moiety in vivo.

Embodiment 164. The glycoengineered polypeptide of embodiment 163, wherein the conjugation occurs in a cell.

Embodiment 165. The glycoengineered polypeptide of embodiment 164, wherein the cell is a leishmania cell.

Embodiment 166. The glycoengineered polypeptide of any one of embodiments 1-162, wherein the second moiety is conjugated to the first moiety by chemical conjugation.

Embodiment 167. The glycoengineered polypeptide of embodiment 166, wherein chemical conjugation comprises click chemistry.

Embodiment 168. The glycoengineered polypeptide of any one of the preceding embodiments, wherein the first moiety is an antibody and the antibody is a monoclonal or polyclonal antibody.

Embodiment 169. The glycoengineered polypeptide of embodiment 168, wherein the antibody is a recombinant antibody.

Embodiment 170. The glycoengineered polypeptide of embodiment 169, wherein the antibody is humanized, chimeric or fully human.

Embodiment 171. The glycoengineered polypeptide of any one of embodiments 168-170, wherein the antibody has a glycan to protein ratio of 2 to 1, 4 to 1, 6 to 1, 8 to 1, or 10 to 1.

Embodiment 172. The glycoengineered polypeptide of embodiment 171, wherein the antibody is glycosylated at a predetermined and specific residue.

Embodiment 173. The glycoengineered polypeptide of any one of the preceding embodiments, characterized in that when administered to a cell, tissue, or subject, the glycoengineered polypeptide which is bound to a target antibody or a complex comprising the same via the first moiety and to an endocytic receptor via a second moiety results in degradation of the target antibody or a complex comprising the same.

Embodiment 174. The glycoengineered polypeptide of embodiment 173, wherein the target antibody comprises IgA1, or a fragment or a complex thereof.

Embodiment 175. The glycoengineered polypeptide of embodiment 174, wherein a complex comprising IgA1 comprises an immune complex.

Embodiment 176. The glycoengineered polypeptide of embodiment 173, wherein the target antibody comprises gd-IgA1 or a fragment or a complex thereof.

Embodiment 177. The glycoengineered polypeptide of embodiment 176, wherein a complex comprising gd-IgA1 comprises an immune complex.

Embodiment 178. The glycoengineered polypeptide of embodiment 173, wherein the target antibody comprises an anti-gd-IgA1 autoantibody, or a fragment or a complex thereof.

Embodiment 179. The glycoengineered polypeptide of embodiment 178, wherein a complex comprising an anti-gd-IgA1 autoantibody comprises an immune complex.

Embodiment 180. The glycoengineered polypeptide of any one of embodiments 173-179, wherein degradation comprises internalization into a cell.

Embodiment 181. The glycoengineered polypeptide of embodiment 180, wherein the target antibody or an immune complex comprising the same, and the glycoengineered polypeptide are internalized.

Embodiment 182. The glycoengineered polypeptide of embodiment 180 or 181, wherein degradation comprises lysosomal degradation.

Embodiment 183. The glycoengineered polypeptide of any one of embodiments 173-182, wherein degradation occurs in a liver cell.

Embodiment 184. The glycoengineered polypeptide of any one of the preceding embodiments, characterized in that when administered to a cell, tissue, or subject, the glycoengineered polypeptide which is bound to a target antibody via the first moiety prevents activation of a complement system.

Embodiment 185. The glycoengineered polypeptide of embodiment 184, wherein a complement system comprises: a lectin pathway, an alternative pathway, or a classical pathway, or a combination thereof.

Embodiment 186. The glycoengineered polypeptide of any one of the preceding embodiments, characterized in that when administered to a cell, tissue, or subject, the glycoengineered polypeptide which is bound to a target antibody via the first moiety prevents and/or reduces formation of an immune complex comprising a target antibody.

Embodiment 187. The glycoengineered polypeptide of any one of the preceding embodiments, characterized in that when administered to a cell, tissue, or subject, the glycoengineered polypeptide which is bound to a target antibody via the first moiety prevents and/or reduces IgA deposits in one or more tissues or organs.

Embodiment 188. A polynucleotide encoding the glycoengineered polypeptide of any one of the preceding embodiments.

Embodiment 189. The polynucleotide of embodiment 188, wherein the polynucleotide comprises a portion of the nucleotide sequence of SEQ ID NO: 6 or a codon-optimized version thereof.

Embodiment 190. The polynucleotide of embodiment 189, wherein the polynucleotide comprises at least 10% of SEQ ID NO: 6, or a codon-optimized version thereof.

Embodiment 191. A composition comprising a glycoengineered polypeptide of any one of the preceding embodiments.

Embodiment 192. The composition of embodiment 191, wherein the composition comprises a glycoengineered polypeptide comprising a first moiety that specifically binds to IgA1 or a fragment or a complex thereof.

Embodiment 193. The composition of embodiment 191, wherein the composition comprises a glycoengineered polypeptide comprising a first moiety that specifically binds to gd-IgA1 or a fragment or a complex thereof.

Embodiment 194. The composition of embodiment 191, wherein the composition comprises a glycoengineered polypeptide comprising a first moiety that specifically binds to an anti-gd-IgA1 autoantibody or a fragment or a complex thereof.

Embodiment 195. The composition of embodiment 191, wherein the composition comprises:
(i) a first glycoengineered polypeptide comprising a first moiety that specifically binds to IgA1 or a fragment or a complex thereof,
(ii) a second glycoengineered polypeptide comprising a first moiety that specifically binds to gd-IgA1 or a fragment or a complex thereof; and/or
(iii) a third glycoengineered comprising a first moiety that specifically binds to an anti-gd-IgA1 autoantibody or a fragment or a complex thereof

Embodiment 196. A composition comprising a population of glycoengineered polypeptides of any one of embodiments 1-190, wherein the population of glycoengineered polypeptides has an N-glycan profile that is at least 30%, at least 40%, at least 50%, at least 60%, at least 70%, at least 80%, at least 90%, at least 95%, at least 98%, or about 100% homogeneous at one or more of the N-glycosylation site(s).

Embodiment 197. The composition of embodiment 196, wherein the homogeneity of the N-glycan profile at one or more of the N-glycosylation sites is determined by N-glycan analysis, glycopeptide analysis or intact protein analysis.

Embodiment 198. The composition of embodiment 196, wherein the N-glycan profile comprises about 30% to 40%, about 40% to about 50%, about 50% to about 60%, about 60% to about 70%, about 70% to about 80%, about 80% to about 90%, or about 90% to about 100% of the N-glycan of the structure provided in embodiment 21.

Embodiment 199. The composition of embodiment 196, has an N-glycan profile comprising about 30% to about 40%, about 40% to about 50%, about 50% to about 60%, about 60% to about 70%, about 70% to about 80%, about 80% to about 90%, or about 90% to about 100% of the N-glycan of the structure provided in embodiment 21 among all glycans in the N-glycan profile.

Embodiment 200. The composition of any one of embodiments 191-199, wherein the composition is a pharmaceutical composition.

Embodiment 201. The composition of embodiment 200, wherein the pharmaceutical composition comprises one or more excipients.

Embodiment 202. A Leishmania host cell expressing a glycoengineered polypeptide of any one of embodiments 1-190.

Embodiment 203. The host cell of embodiment 202, wherein the cell comprises a polynucleotide sequence encoding a glycoengineered polypeptide.

Embodiment 204. A method comprising: administering to a subject a pharmaceutical composition of embodiment 200 or 201.

Embodiment 205. The method of embodiment 204, wherein the subject has or is diagnosed as having a disease associated with increased and/or aberrant IgA.

Embodiment 206. The method of embodiment 205, wherein the disease associated with increased and/or aberrant IgA is IgA nephropathy.

Embodiment 207. The method of embodiment 205, wherein the disease associated with increased and/or aberrant IgA is dermatitis herpetiformis.

Embodiment 208. The method of embodiment 205, wherein the disease associated with increased and/or aberrant IgA is and Henoch-Schoenlein purpura.

Embodiment 209. The method of any one of embodiments 204-208, wherein the method is a treatment method.

Embodiment 210. The method of any one of embodiments 204-208, wherein the method is a prevention method.

Embodiment 211. A method of treating and/or preventing IgA nephropathy (IgAN) in a subject, the method comprising, administering to a subject a pharmaceutical composition of embodiment 200 or 201.

Embodiment 212. The method of any one of embodiments 204-210, wherein the glycoengineered polypeptide is capable of simultaneously binding to the target antibody with the first moiety and binding to an endocytic receptor-expressing cell with the second moiety, thereby causing the target antibody or immune complexes comprising the same to be internalized into a cell.

Embodiment 213. The method of embodiment 212, wherein internalization comprises transporting to a lysosome and/or degradation.

Embodiment 214. The method of embodiment 212 or 213, wherein the endocytic receptor is ASGPR or a variant or fragment thereof.

Embodiment 215. The method of any one of embodiments 204-214, wherein the subject has increased levels of IgA1 or immune complexes comprising the same as compared to a subject who does not have IgAN.

Embodiment 216. The method of embodiment 215, wherein administration of the pharmaceutical composition reduces a level IgA1 or immune complexes comprising the same as compared to a subject who has not been administered the pharmaceutical composition or as compared to the same subject prior to administration of the pharmaceutical composition.

Embodiment 217. The method of embodiment 216, wherein a reduction in level of IgA1 or immune complexes comprising the same comprises degradation of IgA1 or immune complexes comprising the same.

Embodiment 218. The method of embodiment 216 or 217, wherein a reduction in the level of IgA1 or immune complexes comprising the same is a result of internalization into a cell.

Embodiment 219. The method of embodiment 218, wherein internalization comprises transporting to a lysosome and/or degradation.

Embodiment 220. The method of any one of embodiments 204-219, wherein administration of the pharmaceutical composition prevents IgA1 from binding to an antigen or a binding partner.

Embodiment 221. The method of embodiment 220, wherein the IgA1 binding partner is CD89 or a fragment thereof.

Embodiment 222. The method any one of embodiments 204-220, wherein administration of the pharmaceutical composition prevents and/or reduces the formation of an immune complex comprising IgA1.

Embodiment 223. The method of any one of embodiments 204-222, wherein the subject has increased levels of gd-IgA1 or immune complexes comprising the same as compared to a subject who does not have IgAN.

Embodiment 224. The method of embodiment 223, wherein administration of the pharmaceutical composition reduces a level gd-IgA1 or immune complexes comprising the same as compared to a subject who has not been administered the pharmaceutical composition or as compared to the same subject prior to administration of the pharmaceutical composition.

Embodiment 225. The method of embodiment 224, wherein a reduction in level of gd-IgA1 or immune complexes comprising the same comprises degradation of gd-IgA1 or immune complexes comprising the same.

Embodiment 226. The method of embodiment 224 or 225, wherein a reduction in the level of gd-IgA1 or immune complexes comprising the same is a result of internalization into a cell.

Embodiment 227. The method of embodiment 226, wherein internalization comprises transporting to a lysosome and/or degradation.

Embodiment 228. The method of any one of embodiments 204-227, wherein administration of the pharmaceutical composition prevents gd-IgA1 from binding to an antigen or a binding partner.

Embodiment 229. The method of any one of embodiments 204-228, wherein administration of the pharmaceutical composition prevents and/or reduces the formation of an immune complex comprising gd-IgA1.

Embodiment 230. The method of any one of embodiments 204-229, wherein the subject has detectable levels of anti-gd-IgA1 or immune complexes comprising the same as compared to a subject who does not have IgAN.

Embodiment 231. The method of embodiment 230, wherein administration of the pharmaceutical composition reduces a level anti-gd-IgA1 or immune complexes comprising the same as compared to a subject who has not been administered the pharmaceutical composition or as compared to the same subject prior to administration of the pharmaceutical composition.

Embodiment 232. The method of embodiment 231, wherein a reduction in level of gd-IgA1 or immune complexes comprising the same comprises degradation of gd-IgA1 or immune complexes comprising the same.

Embodiment 233. The method of embodiment 231 or 232, wherein a reduction in the level of anti-gd-IgA1 or immune complexes comprising the same is a result of internalization into a cell.

Embodiment 234. The method of embodiment 233, wherein internalization comprises transporting to a lysosome and/or degradation.

Embodiment 235. The method of any one of embodiments 204-234, wherein administration of the pharmaceutical composition prevents anti-gd-IgA1 from binding to an antigen or a binding partner.

Embodiment 236. The method of any one of embodiments 204-235, wherein administration of the pharmaceutical composition prevents and/or reduces the formation of an immune complex comprising anti-gd-IgA1.

Embodiment 237. The method of any one of embodiments 204-236, wherein administration of the pharmaceutical composition reduces activation of a complement system as compared to a subject who has not been administered the pharmaceutical composition or as compared to the same subject prior to administration of the pharmaceutical composition.

Embodiment 238. The method of embodiment 237 wherein a complement system comprises: a lectin pathway, an alternative pathway, or a classical pathway, or a combination thereof.

Embodiment 239. The method of embodiment 237 or 238, wherein a complement system comprises C3, C5b, C6, C7, C8, and/or C9, or fragments of any complement component or combinations thereof.

Embodiment 240. The method of any one of embodiments 237-239, wherein complement system comprises C3 or fragments of C3.

Embodiment 241. The method of embodiment 240, wherein the C3 fragments comprise iC3b, C3c, C3dg, or combinations thereof.

Embodiment 242. The method of any one of embodiments 216-241, wherein the reduction in (i) IgA1 level, (ii) gd-IgA1 level, (iii) anti-gd-IgA1 level, (iv) levels of immune complexes comprising IgA1, gd-IgA1 and/or anti-gd-IgA1, or (iv) complement activation is assessed in a sample from the subject.

Embodiment 243. The method of any one of embodiments 204-242, wherein administration of the pharmaceutical composition prevents and/or reduces IgA deposits in the kidney of a subject as compared to a subject who has not been administered the pharmaceutical composition or as compared to the same subject prior to administration of the pharmaceutical composition.

Embodiment 244. The method of any one of embodiments 204-243, wherein administration of the pharmaceutical composition treats and/or prevents the disease.

Embodiment 245. The method of any one of embodiments 207-244, wherein administration of the pharmaceutical composition alleviates one or more symptoms of the disease.

Embodiment 246. A method comprising, assessing a level of a target antibody in a sample from a subject, and administering a pharmaceutical composition of any one of embodiments 200 or 201 if the level of the target antibody is higher than a comparator.

Embodiment 247. The method of embodiment 246, wherein the comparator comprises a predetermined reference sample such as a sample obtained from an otherwise similar subject who does not have a disease or disorder, or a symptom of a disease or disorder.

Embodiment 248. The method of embodiment 247, wherein the disease or disorder is IgA nephropathy.

Embodiment 249. The method of any one of embodiments 246-248, wherein the method is a treatment method.

Embodiment 250. The method of any one of embodiments 246-248, wherein the method is a prevention method.

Embodiment 251. The method of any one of embodiments 246-250, wherein the level of target antibody is assessed using an assay that detects the level and/or activity of IgA1, gd-IgA1, or anti-gd-IgA1, or a combination thereof.

Embodiment 252. The method of embodiment 251, wherein the assay is a dot blot assay.

Embodiment 253. The method of embodiment 251, wherein the assay is a capture ELISA.

Embodiment 254. A method treating and/or preventing IgA nephropathy, comprising:
(a) determining a level of a target antibody in a sample from a subject, wherein:
   (i) a higher level of the target antibody in the sample as compared to a comparator indicates that the subject has or is at risk of developing IgaN; and
   (ii) a lower level of the target antibody in the sample as compared to a comparator indicates that the subject does not have or is not as risk of developing IgaN; and
(b) responsive to said determination, administering a pharmaceutical composition of embodiment 200 or 201 to the subject.

Embodiment 255. The method of embodiment 254, wherein the comparator comprises a predetermined reference sample such as a sample obtained from an otherwise similar subject who does not have IgAN.

Embodiment 256. The method of embodiment 254 or 255, wherein the subject having a higher level of the target antibody is administered the pharmaceutical composition.

Embodiment 257. The method of embodiment 254 or 255, wherein the subject having a lower level of the target antibody is not administered the pharmaceutical composition.

Embodiment 258. The method of any one of embodiments 254-257, wherein the method is a treatment method.

Embodiment 259. The method of any one of embodiments 254-257, wherein the method is a prevention method.

Embodiment 260. The method of any one of embodiments 254-259, wherein the level of target antibody is assessed using an assay that detects the level and/or activity of IgA1, gd-IgA1, or anti-gd-IgA1, or a combination thereof.

Embodiment 261. The method of embodiment 260, wherein the assay is a dot blot assay.

Embodiment 262. The method of embodiment 260, wherein the assay is a capture ELISA.

Embodiment 263. The method of any one of embodiments 204-262, wherein the method comprises administering the pharmaceutical composition once.

Embodiment 264. The method of any one of embodiments 204-262, wherein the method comprises administering the pharmaceutical composition repeatedly.

Embodiment 265. The method of any one of embodiments 204-264, wherein the method comprises administering the pharmaceutical composition in combination with one or more additional therapies.

Embodiment 266. The method of embodiment 265, wherein the one or more additional therapies comprises an agent that inhibits activation of a complement system, an agent that treats proteinuria, an agent that treats blood pressure, a renal protectant, or combinations thereof.

Embodiment 267. The method of any one of embodiments 204-266, wherein the administration step comprises intravenous injection, intraperitoneal injection, subcutaneous injection, transdermal injection, or intramuscular injection.

Embodiment 268. The method of any one of embodiments 204-267, wherein the subject is a mammal.

Embodiment 269. The method of embodiment 268, wherein the subject is a human.

### INCORPORATION BY REFERENCE

Each publication, including scientific references to the scientific literature, patent references, and electronic databases, websites and other references accessible through the internet, are hereby incorporated by reference herein, in their entirety, for their disclosure relevant to this specification and as cited herein.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

What is claimed is:

1. A glycoengineered polypeptide comprising:
(a) a first moiety comprising one or more peptides that specifically binds to a target antibody or a fragment or a complex thereof; and
(b) a second moiety comprising one or more glycans conjugated to the first moiety at one or more glycosylation sites,
wherein the target antibody comprises:
(i) galactose-deficient IgA1 (gd-IgA1), or a fragment or a complex thereof;
(ii) IgA1, or a fragment or a complex thereof; or
(iii) an autoantibody that specifically binds to gd-IgA1 (“anti-gd-IgA1 autoantibody”), or a fragment or a complex thereof.

2. The glycoengineered polypeptide of claim 1, wherein the second moiety specifically binds to one or more endocytic receptors.

3. The glycoengineered polypeptide of claim 2, wherein the endocytic receptor is chosen from: an asialoglycoprotein receptor (ASGPR); a mannose binding receptor, a Cluster of Differentiation 206 (CD206) receptor; a DC-SIGN (Cluster of Differentiation 209 or CD209) receptor; a C-Type Lectin Domain Family 4 Member G (LSECTin) receptor; a macrophage inducible Ca2+-dependent lectin receptor (Mincle); a L-SIGN CD209L receptor; dectin-1; dectin-2, langerin, macrophage mannose 2 receptor, BDCA-2, DCIR, MBL, MDL, MICL, CLEC2, CLEC10, DNGR1, CLEC12B, DEC-205, and mannose 6 phosphate receptor (M6PR), or a combination thereof.

4. The glycoengineered polypeptide of any one of the preceding claims, wherein the one or more glycans comprise a terminal GlcNac, a terminal GalNac, or a terminal Gal.

5. The glycoengineered polypeptide of any one of the preceding claims, wherein the one or more glycans is an N-glycan, optionally wherein the N-glycan is linked to the first moiety of the glycoengineered polypeptide at 1, 2, 3, 4 or 5 N-glycosylation sites.

6. The glycoengineered polypeptide of any one of the preceding claims, wherein the one or more glycans comprise a glycan structure comprising GlcNAc2-Man3-GlcNAc2, GalNAc2-GlcNAc2-Man3-GlcNAc2, Gal2-GlcNAc2-Man3-GlcNAc2, GlcNAc1-Man3-GlcNAc2, Gal2-GlcNAc2-Man3-GlcNAc2, Gal1-GlcNAc2-Man3-GlcNAc2, GalNAc1-GlcNAc2-Man3-GlcNAc2, GlcNAc3-Man3-GlcNAc2, GlcNAc4-Man3-GlcNAc2, Gal3-GlcNAc3-Man3-GlcNAc2, GalNAc3-GlcNAc3-Man3-GlcNAc2, GalNAc4-GlcNAc4-Man3-GlcNAc2, Gal4-GlcNAc4-Man3-GlcNAc2, or Man-6-P-N-glycan.

7. The glycoengineered polypeptide of claim 6, wherein the glycan structure comprises a monoantennary structure, biantennary structure, a triantennary structure, or a tetraantennary structure.

8. The glycoengineered polypeptide of claim 6 or 7, wherein the glycan structure comprises a biantennary structure, optionally wherein the glycan structure comprises a biantennary GalNAc.

9. The glycoengineered polypeptide of claim 8, wherein the biantennary GalNac binds to an asialoglycoprotein receptor (ASGPR) or a fragment or variant thereof, or a complex comprising ASGPR.

10. The glycoengineered polypeptide of any one of claims 5-9, wherein the N-glycan has a structure of: 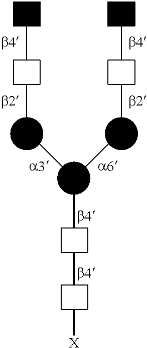
wherein the black square represents an N-acetyl galactosamine (GalNAc), the white square represents an N-acetylglucosamine (GlcNAc) residue and the black circle represents a mannose (Man) residue, and wherein X represents an amino acid residue of the first moiety.

11. The glycoengineered polypeptide of any one of claims 5-10, wherein the N-glycan is conjugated to the glycoengineered polypeptide at least one, two, three, or four N-glycosylation sites.

12. The glycoengineered polypeptide of any one of claims 5-11, wherein the N-glycosylation site comprises a consensus sequence of N-X-S/T or N-X-C, wherein X is any amino acid except proline.

13. The glycoengineered polypeptide of any one of claims 5-12, wherein the N-glycosylation site is naturally occurring.

14. The glycoengineered polypeptide of any one of claims 5-12, wherein the N-glycosylation site is engineered into the amino acid sequence of the first moiety, optionally wherein the engineered N-glycosylation is SEQ ID NO: 37.

15. The glycoengineered polypeptide of any one of claims 2-14, wherein the endocytic receptor is or comprises ASGPR or a fragment or variant thereof, or a complex comprising ASGPR, optionally wherein when the endocytic receptor is ASGPR, the glycan structure of the second moiety comprises a terminal GalNac.

16. The glycoengineered polypeptide of any one of the preceding claims, wherein the first moiety comprises one or more peptides that specifically bind to a glycosylation-deficient IgA1 (gd-IgA1), or a fragment or a complex thereof, optionally wherein the gd-IgA1 comprises a hinge region that is glycosylated.

17. The glycoengineered polypeptide of claim 16, wherein the one or more peptides that specifically bind to gd-IgA1 recognize a glycan profile on gd-IgA1 that is different from a reference glycan profile of an IgA1.

18. The glycoengineered polypeptide of claim 17, wherein the reference glycan profile comprises an O-glycosylation profile, optionally wherein the reference O-glycosylation profile comprises N-acetylgalactosamine (GalNac).

19. The glycoengineered polypeptide of claim 17 or 18, wherein the gd-IgA1 glycan profile comprises a terminal GalNac (also referred to as the Tn antigen).

20. The glycoengineered polypeptide of claim 17 or 18, wherein the gd-IgA1 glycan profile comprises GalNac with an alpha2,6 linked sialic acid (also referred to as the STn antigen).

21. The glycoengineered polypeptide of any one of claims 16-20, wherein the one or more peptides that specifically bind to gd-IgA1 comprises an antibody agent. comprising an antigen binding fragment.

22. The glycoengineered polypeptide of claim 21, wherein the antibody agent comprises a full antibody, a Fab fragment, an scFv, a nanobody, a duobody, a single domain antibody (e.g., a VHH).

23. The glycoengineered polypeptide of any one of claims 1-15, wherein the first moiety comprises one or more peptides that specifically bind to IgA1, or a fragment or a complex thereof.

24. The glycoengineered polypeptide of claim 23, wherein the one or more peptides that specifically bind to IgA1 or a fragment thereof are each conjugated to a second moiety.

25. The glycoengineered polypeptide of claim 23 or 24, wherein an IgA1 amino acid sequence is provided as SEQ ID NO: 2.

26. The glycoengineered polypeptide of any one of claims 23-25, wherein the one or more peptides that specifically bind to IgA1 recognize an epitope on IgA1.

27. The glycoengineered polypeptide of any one of claims 23-26, wherein the first moiety comprising one or more peptides that specifically bind to IgA1, or a fragment or a complex thereof comprises a CD89 (FcaR1) polypeptide, or a variant, or a fragment thereof.

28. The glycoengineered polypeptide of claim 27, wherein the CD89 comprises soluble CD89 or a fragment or variant thereof.

29. The glycoengineered polypeptide of claim 27 or 28, wherein a CD89 polypeptide is provided as SEQ ID NO: 5 (e.g., with or without the signal peptide), SEQ ID NO: 9 (e.g., with or without the signal peptide), SEQ ID NO: 38 (e.g., with or without the signal peptide), or SEQ ID NO: 39.

30. The glycoengineered polypeptide of any one of claims 27-29, wherein the one or more peptides that specifically bind to IgA1, or a fragment or a complex thereof comprises a fragment of a CD89 polypeptide.

31. The glycoengineered polypeptide of claim 30, wherein the one or more peptides comprises at least 5% of a full length CD89 polypeptide, or a CD89 polypeptide sequence provided in SEQ ID NO: 5 (e.g., with or without the signal peptide), SEQ ID NO: 9 (e.g., with or without the signal peptide), SEQ ID NO: 38 (e.g., with or without the signal peptide), or SEQ ID NO: 39.

32. The glycoengineered polypeptide of any one of claims 27-31, wherein the fragment comprises one or more additional amino acid sequences 5′ and/or 3′ to the fragment sequence.

33. The glycoengineered polypeptide of any one of claims 27-32, wherein the one or more peptides that specifically bind to IgA1, or a fragment or a complex thereof comprises a variant of a CD89 polypeptide.

34. The glycoengineered polypeptide of any one of claims 27-33, wherein the one or more peptides that specifically bind to IgA1, or a fragment or a complex thereof comprises a sequence having at least 85% identity to a CD89 polypeptide sequence provided in:
(i) SEQ ID NO: 5 with or without the signal peptide of SEQ ID NO: 44,
(ii) SEQ ID NO: 9 with or without the signal peptide of SEQ ID NO: 44,
(ii) SEQ ID NO: 38 with or without the signal peptide of SEQ ID NO: 42, or
(iv) SEQ ID NO: 39.

35. The glycoengineered polypeptide of any one of claims 23-34, wherein the one or more peptides that specifically bind to IgA1 comprises a sequence having at least 85% identity to SEQ ID NO: 41.

36. The glycoengineered polypeptide of any one of claims 23-35, wherein the one or more peptides that specifically bind to IgA1 comprises an antibody agent comprising an antigen binding fragment.

37. The glycoengineered polypeptide of claim 36, wherein the antibody agent comprises a full antibody, a Fab fragment, an scFv, a nanobody, a duobody, a single domain antibody (e.g., a VHH).

38. The glycoengineered polypeptide of any one of claims 1-15, wherein the first moiety comprises one or more peptides that specifically bind to an anti-gd-IgA1 autoantibody.

39. The glycoengineered polypeptide of claim 38, wherein the one or more peptides that specifically bind to anti-gd-IgA1 or a fragment thereof are each conjugated to a second moiety.

40. The glycoengineered polypeptide of claim 38 or 39, wherein the anti-gd-IgA1 autoantibody is an IgG, an IgM, an IgE, an IgD, or an IgM.

41. The glycoengineered polypeptide of any one of claims 38-40, wherein the anti-gd-IgA1 autoantibody is an IgG comprising a mutation in a complementarity determining region 3 (CDR3) of an Ig heavy chain (IgH) variable region.

42. The glycoengineered polypeptide of any one of claims 38-41, wherein the first moiety comprises one or more peptides that specifically bind to a mutation in a CDR3 IgH region an anti-gd-IgA1 autoantibody.

43. The glycoengineered polypeptide of any one of claims 38-42, wherein the one or more peptides that specifically bind to an anti-gd-IgA1 autoantibody binds to an IgG protein, or a fragment or a variant thereof.

44. The glycoengineered polypeptide of any one of claims 38-43, wherein the first moiety comprising one or more peptides that specifically bind to an anti-gd-IgA1 autoantibody comprises a gd-IgA1 polypeptide, or a variant, or fragment thereof.

45. The glycoengineered polypeptide of claim 44, wherein the gd-IgA1 comprises a hinge region that is glycosylated.

46. The glycoengineered polypeptide of claim 45, wherein the first moiety comprising one or more peptides that specifically bind to an anti-gd-IgA1 autoantibody comprises a gd-IgA1 polypeptide fragment optionally, wherein the fragment comprises the sequence of SEQ ID NO: 1, or a fragment or a variant thereof.

47. The glycoengineered polypeptide of claim 46, wherein the fragment comprises one or more additional amino acid residues to the 5′ and/or 3′ end of the sequence.

48. The glycoengineered polypeptide of any one of claims 38-47, wherein the one or more peptides that specifically bind to anti-gd-IgA1 comprises an antibody agent comprising an antigen binding fragment.

49. The glycoengineered polypeptide of claim 48, wherein the antibody agent comprises a full antibody, a Fab fragment, an scFv, a nanobody, a duobody, a single domain antibody (e.g., a VHH).

50. The glycoengineered polypeptide of any one of the preceding claims, wherein the first moiety comprises:
(i) one or more peptides that specifically bind to gd-IgA1 or a fragment or complex thereof;
(ii) one or more peptides that specifically bind to IgA1 or a fragment or complex thereof;
(iii) one or more peptides that specifically bind to an anti-gd-IgA1 autoantibody or a fragment or complex thereof; or
(iv) any one or all of (i)-(iii).

51. The glycoengineered polypeptide of any one of the preceding claims, wherein the polypeptide comprises one or more additional elements chosen from:
(a) a linker,
(b) a spacer,
(c) a cleavage peptide, e.g., an IRES or a protease cleavage site,
(d) a signal peptide,
(e) a tag, e.g., a cleavable tag,
(f) a half-life extender domain, e.g., an Fc domain or albumin, or
(g) any combination of (a)-(f).

52. The glycoengineered polypeptide of any one of the preceding claims, wherein the second moiety is conjugated to the first moiety in vivo.

53. The glycoengineered polypeptide of claim 52, wherein the conjugation occurs in a cell, optionally wherein the cell is a Leishmania cell.

54. The glycoengineered polypeptide of any one of claims 1-52, wherein the second moiety is conjugated to the first moiety by chemical conjugation, optionally wherein chemical conjugation comprises click chemistry.

55. A polynucleotide encoding the glycoengineered polypeptide of any one of the preceding claims.

56. A composition comprising a glycoengineered polypeptide of any one of claims 1-54.

57. A composition comprising a population of glycoengineered polypeptides of any one of claims 1-54, wherein the population of glycoengineered polypeptides has an N-glycan profile that is at least 30% homogeneous at one or more of the N-glycosylation site(s).

58. The composition of claim 57, wherein the N-glycan profile comprises about 30% of the N-glycan of the structure provided in claim 10.

59. The composition of any one of claims 56-58, wherein the composition is a pharmaceutical composition.

60. A Leishmania host cell expressing a glycoengineered polypeptide of any one of claims 1-54, wherein the cell comprises a polynucleotide sequence encoding a glycoengineered polypeptide.

61. A method comprising:
administering to a subject a pharmaceutical composition of claim 59.

62. The method of claim 61, wherein the subject has or is diagnosed as having a disease associated with increased and/or aberrant IgA.

63. The method of claim 62, wherein the disease associated with increased and/or aberrant IgA is IgA nephropathy, dermatitis herpetiformis, or Henoch-Schoenlein purpura.

64. The method of any one of claims 61-63, wherein the method is a treatment method or a prevention method.

65. A method of treating and/or preventing IgA nephropathy (IgAN) in a subject, the method comprising, administering to a subject a pharmaceutical composition of claim 59.

66. The method of any one of claims 61-65, wherein the glycoengineered polypeptide is capable of simultaneously binding to the target antibody with the first moiety and binding to an endocytic receptor-expressing cell with the second moiety, thereby causing the target antibody or immune complexes comprising the same to be internalized into a cell.

67. The method of claim 66, wherein internalization comprises transporting to a lysosome and/or degradation.

68. The method of claim 66 or 67, wherein the endocytic receptor is ASGPR or a variant or fragment thereof.

69. The method of any one of claims 61-68, wherein the subject has increased levels of IgA1 or immune complexes comprising the same as compared to a subject who does not have IgAN.

70. The method of claim 69, wherein administration of the pharmaceutical composition reduces a level IgA1 or immune complexes comprising the same as compared to a subject who has not been administered the pharmaceutical composition or as compared to the same subject prior to administration of the pharmaceutical composition, optionally wherein a reduction in level of IgA1 or immune complexes comprising the same comprises degradation of IgA1 or immune complexes comprising the same.

71. The method of any one of claims 61-70, wherein administration of the pharmaceutical composition:
(i) prevents IgA1 from binding to an antigen or a binding partner, optionally, wherein the IgA1 binding partner is CD89 or a fragment thereof; or
(ii) prevents and/or reduces the formation of an immune complex comprising IgA1.

72. The method of any one of claims 61-71, wherein administration of the pharmaceutical composition:
(i) prevents gd-IgA1 from binding to an antigen or a binding partner; or
(ii) prevents and/or reduces the formation of an immune complex comprising gd-IgA1.

73. The method of any one of claims 61-72, wherein administration of the pharmaceutical composition:
(i) prevents anti-gd-IgA1 from binding to an antigen or a binding partner; or
(ii) prevents and/or reduces the formation of an immune complex comprising anti-gd-IgA1.

74. The method of any one of claims 61-73, wherein administration of the pharmaceutical composition reduces activation of a complement system as compared to a subject who has not been administered the pharmaceutical composition or as compared to the same subject prior to administration of the pharmaceutical composition, optionally wherein a complement system comprises: a lectin pathway, an alternative pathway, or a classical pathway, or a combination thereof.

75. The method of claim 74, wherein a complement system comprises C3, C5b, C6, C7, C8, and/or C9, or fragments of any complement component or combinations thereof.

76. The method of any one of claims 61-75, wherein administration of the pharmaceutical composition treats and/or prevents the disease.

77. The method of any one of claims 61-76, wherein administration of the pharmaceutical composition alleviates one or more symptoms of the disease.

78. A method comprising,
assessing a level of a target antibody in a sample from a subject, and
administering a pharmaceutical composition of claim 59 if the level of the target antibody is higher than a comparator.

79. The method of any one of claims 61-78, wherein the method comprises administering the pharmaceutical composition in combination with one or more additional therapies, optionally wherein the one or more additional therapies comprises an agent that inhibits activation of a complement system, an agent that treats proteinuria, an agent that treats blood pressure, a renal protectant, or combinations thereof.

80. The method of any one of claims 61-79, wherein the administration step comprises intravenous injection, intraperitoneal injection, subcutaneous injection, transdermal injection, or intramuscular injection.

81. The method of any one of claims 61-80, wherein the subject is a mammal.


SEQUENCE LISTING (48 sequences)

SEQ ID NO: 1 - protein, 21 aa, synthetic construct
PVPSTPPTPSPSTPPTPSPSC

SEQ ID NO: 2 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 3 - protein, 330 aa, synthetic construct [sequence omitted]

SEQ ID NO: 4 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 287 aa, Homo sapiens
MDPKQTTLLCLVLCLGQRIQAQEGDFPMPFISAKSSPVIPLDGSVKIQCQAIREAYLTQLMIIKNSTYREIGRRLKFWNETDPEFVIDHMDANKAGRYQCQYRIGHYRFRYSDTLELVVTGLYGKPFLSADRGLVLMPGENISLTCSSAHIPFDRFSLAKEGELSLPQHQSGEHPANFSLGPVDLNVSGIYRCYGWYNRSPYLWSFPSNALELVVTDSIHQDYTTQNLIRMAVAGLVLVALLAILVENWHSHTALNKEASADVAEPSWSQQMCQPGLTFARTPSVCK

SEQ ID NO: 6-7 - skipped (2 entries)

SEQ ID NO: 8 - protein, 19 aa, synthetic construct
VPSTPPTPSPSTPPTPSPS

SEQ ID NO: 9 - protein, 217 aa, Homo sapiens
MDPKQTTLLCLVLCLGQRIQAQEGRYISEHFWCRSLGCNPVNDASAQRPGDFPMPFISAKSSPVIPLDGSVKIQCQAIREAYLTQLMIIKNSTYREIGRRLKFWNETDPEFVIDHMDANKAGRYQCQYRIGHYRFRYSDTLELVVTDSIHQDYTTQNLIRMAVAGLVLVALLAILVENWHSHTALNKEASADVAEPSWSQQMCQPGLTFARTPSVCK

SEQ ID NO: 10-20 - skipped (11 entries)

SEQ ID NO: 21 - protein, 25 aa, synthetic construct
MIASSVRHAVILLLVAVAMMAAVIA

SEQ ID NO: 22 - protein, 26 aa, unidentified
  source 1..26: note = Leishmania tarentolae invertase
MIASSVRHAVILLLVAVAMMAAAVIA

SEQ ID NO: 23-36 - skipped (14 entries)

SEQ ID NO: 37 - protein, 15 aa, synthetic construct
GGGGANSTAPAPAPA

SEQ ID NO: 38 - protein, 220 aa, synthetic construct
MIASSVRHAVILLLVAVAMMGGVIAQEGDFPMPFISAKSSPVIPLDGSVKIQCQAIREAYLTQLMIIKNSTYREIGRRLKFWNETDPEFVIDHMDANKAGRYQCQYRIGHYRFRYSDTLELVVTGLYGKPFLSADRGLVLMPGEQISLTCSSAHIPFDRFSLAKEGELSLPQHQSGEHPAQFSLGPVDLQVSGIYRCYGWYQRSPYLWSFPSNALELVVT

SEQ ID NO: 39 - protein, 195 aa, synthetic construct
QEGDFPMPFISAKSSPVIPLDGSVKIQCQAIREAYLTQLMIIKNSTYREIGRRLKFWNETDPEFVIDHMDANKAGRYQCQYRIGHYRFRYSDTLELVVTGLYGKPFLSADRGLVLMPGEQISLTCSSAHIPFDRFSLAKEGELSLPQHQSGEHPAQFSLGPVDLQVSGIYRCYGWYQRSPYLWSFPSNALELVVT

SEQ ID NO: 40 - protein, 10 aa, synthetic construct
HHHHHHHHHH

SEQ ID NO: 41 - protein, 245 aa, synthetic construct
MIASSVRHAVILLLVAVAMMGGVIAQEGDFPMPFISAKSSPVIPLDGSVKIQCQAIREAYLTQLMIIKNSTYREIGRRLKFWNETDPEFVIDHMDANKAGRYQCQYRIGHYRFRYSDTLELVVTGLYGKPFLSADRGLVLMPGEQISLTCSSAHIPFDRFSLAKEGELSLPQHQSGEHPAQFSLGPVDLQVSGIYRCYGWYQRSPYLWSFPSNALELVVTGGGGANSTAPAPAPAHHHHHHHHHH

SEQ ID NO: 42 - protein, 25 aa, synthetic construct
MIASSVRHAVILLLVAVAMMGGVIA

SEQ ID NO: 43 - protein, 20 aa, unidentified
  source 1..20: note = Leishmania major Alkaline phosphatase
MASRLVRVLAAAMLVAAAVS

SEQ ID NO: 44 - protein, 21 aa, Homo sapiens
MDPKQTTLLCLVLCLGQRIQA

SEQ ID NO: 45 - protein, 5 aa, synthetic construct
EAAAK

SEQ ID NO: 46 - protein, 5 aa, synthetic construct
  REGION >5: note = The entire sequence of amino acids 1-5 can be repeated upto twenty times or absent.
GGGGS

SEQ ID NO: 47 - protein, 4 aa, synthetic construct
YCAR

SEQ ID NO: 48 - protein, 4 aa, synthetic construct
YCAK